(19) United States
(12) Patent Application Publication
(10) Pub. No.: US 2026/0132191 A1
(43) Pub. Date: May 14, 2026

(54) METHODS FOR MODULATING ACTIVITY OF IL-18 FOR TREATMENT OF CD4+ T CELL MEDIATED AUTOIMMUNITY AND IMMUNOPATHOLOGY

(71) Applicant: THE CHILDREN'S HOSPITAL OF PHILADELPHIA, Philadelphia, PA (US)

(72) Inventors: Scott W. CANNA, Bala Cynwyd, PA (US); Vinh DANG, Philadelphia, PA (US); Jeremy A. MORRISSETTE, Philadelphia, PA (US)

(73) Assignee: THE CHILDREN'S HOSPITAL OF PHILADELPHIA, Philadelphia, PA (US)

(21) Appl. No.: 19/446,414

(22) Filed: Jan. 12, 2026

Related U.S. Application Data
(63) Continuation-in-part of application No. PCT/US2024/037468, filed on Jul. 10, 2024.
(60) Provisional application No. 63/512,789, filed on Jul. 10, 2023.

(51) Int. Cl. C07K 16/24; A61K 9/00; A61K 31/7088; A61K 35/17; A61K 38/20; A61K 40/11; A61K 40/15; A61K 45/06; A61P 25/28

(52) U.S. Cl. CPC C07K 16/244; A61K 9/0019; A61K 31/7088; A61K 35/17; A61K 38/20; A61K 40/11; A61K 40/15; A61K 45/06; A61P 25/28; C07K 2317/76

(57) ABSTRACT

Compositions and methods for modulating IL-18 activity for amelioration of aberrant inflammatory symptoms associated with increased autoreactivity of CD4+ T cells are disclosed.

### CROSS-REFERENCE TO RELATED APPLICATIONS

This application is a Continuation in Part application of PCT/US2024/037468 filed Jul. 10, 2024 which claims the benefit of U.S. Provisional Patent Application No. 63/512,789, filed Jul. 10, 2023, the entire contents of each being incorporated herein as though set forth in full.

### GOVERNMENT SUPPORT STATEMENT

This invention was made with government support under grant number R01HD098428 awarded by the National Institutes of Health. The government has certain rights in the invention.

### INCORPORATION-BY-REFERENCE OF MATERIAL SUBMITTED IN ELECTRONIC FORM

The Contents of the electronic sequence listing (CHOP-140-PCT.xml; Size: 122,894 bytes; and Date of Creation: Jul. 10, 2024) is herein incorporated by reference in its entirety.

### FIELD

This invention relates to the fields of immunological and inflammatory disorders, particularly, disorders associated with, or caused by, auto-reactive or otherwise pathogenic T cells (CD4+, CD4−, CD8+, regulatory T cells, natural killer (NK) cells), and antigen presenting cells (APCs) and methods for treatment thereof with agents which enhance or augment the immune-protective activity of the cytokine Interleukin 18 (IL-18).

### BACKGROUND

Several publications and patent documents are cited throughout the specification in order to describe the state of the art to which this invention pertains. Each of these citations is incorporated herein by reference as though set forth in full.

IL-18 is an IL-1 family cytokine with both pro-inflammatory and immune-protective functions. IL-18 is thought to drive and/or exacerbate the “autoinflammatory” category of immune dysregulation disorders associated with extremely elevated levels of IL-18 in circulation. These include Systemic Juvenile Idiopathic Arthritis (sJIA)^{1,2}, Adult-Onset Stills Disease (AOSD)³, Macrophage Activation Syndrome (MAS, often a complication of SJIA or AOSD), and several ultra-rare autoinflammatory Inborn Errors of Immunity (IEI) including diseases associated with gain-of-function mutations in NLRC4^{1,4} or PSTPIP1⁵, deficiency of XIAP⁶, or specific c-terminal mutations in CDC42^{7,8}.

In contrast with autoinflammatory diseases, there are several diseases in which symptoms are caused by pathogenic CD4 T-cells. Diseases wherein CD4+ T cells are likely pathogenic include those with a strong association with specific Major Histocompatibility Complex (MHC) class II alleles (MHCII), i.e. those encoding HLA-DP, HLA-DM, HLA-DOA, HLA-DOB, HLA-DQ, and HLA-DR. There have been extensive studies to understand the association between variations of human leukocyte antigens (HLAs) and various diseases⁹. Clearly a need exists for new agents and treatment modalities to prevent and/or alleviate symptoms caused by pathogenic CD4+ T cells.

Autoimmune diseases with MHCII associations (and thereby likely to be caused by CD4+ T-cells) include but are not limited to multiple sclerosis (MS), rheumatoid arthritis (RA), Sjögren's syndrome, systemic lupus erythematosus (SLE), type 1 diabetes mellitus (T1D), Autoimmune thyroiditis (both Graves and Hashimoto's), autoimmune neutropenia, autoimmune hemolytic anemia, immune thrombocytopenic purpura, cold agglutinin disease, Pemphigus Vulgaris, and Myasthenia Gravis. In addition to defects in HLAs, these autoimmune diseases are also associated with autoreactive or pathogenic antigen specific CD4+ T cells⁹. Similar Inflammatory diseases with MHCII associations where the pathogenic T-cell reactivity may not be to self, include, but are not limited to, ulcerative colitis (UC) and celiac disease (CeD). Pathogenic CD4s are also seen in allergic disorders like atopic dermatitis, allergic rhinosinusitis, asthma, and eosinophilic gastritis/esophagitis as well as in graft-versus-host-diseases (GvHD).

There is a growing amount of literature suggesting the existence of suppressive, regulatory CD8+ T cells that have the capacity to inhibit the function and or proliferation of autoreactive CD4+ T cells to reduce autoimmunity¹². Recently published data from Li et al have described a specific subset of human suppressive CD8s as KIR+CD8+ cells that are the human equivalent to Ly49+CD8+ cells in mice¹³. Our recent experiments in murine models suggest that, in conditions of pathologic CD4 T-cell activation, IL-18 may specifically enhance the number and/or function of suppressive CD8 T-cells and/or regulatory CD4 T-cells to limit/prevent immunopathology mediated by pathogenic CD4 T-cells. Thus, new agents and treatment modalities that mimic the beneficial effects of IL-18 may prevent and/or alleviate symptoms caused by pathogenic CD4+ T cells.

It is an object of the present inventors to provide new treatment modalities to alleviate symptoms caused by pathogenic immune cell signaling.

### SUMMARY

New agents and treatment modalities that utilize the beneficial effects of IL-18 signaling for inhibiting onset of, and/or alleviating symptoms caused fully or partially by pathogenic CD4+ T cells are disclosed. The data presented herein demonstrate that, in a murine model of CD4 T-cell driven autoimmunity, excess IL-18 acts on CD8 T cells to prevent and/or ameliorate symptoms of autoimmunity. Our recent work has established that, in systems where pathogenic autoreactive CD4 T-cells are opposed by suppressor CD8 T-cells, IL-18 may specifically promote the function of the latter to substantially prevent or improve symptoms of autoimmunity. We thus hypothesized that IL-18, and agonists thereof, can be utilized to augment the number and/or function of suppressor cytotoxic cells to prevent autoimmunity by direct or indirect inhibition of pathogenic, autoreactive CD4 T cells.

In certain aspects, a method for inhibiting pathogenic immune cell activities, (e.g., by lymphocytes like CD4+ T-cells, CD8+ T-cells, gamma/delta T-cells, Natural Killer T-cells, and/or cells providing stimulation to these lymphocytes including various antigen presenting cells) and ameliorating disease activity in autoimmune, allergic, or post-transplant disorders in a subject in need thereof is provided. An exemplary method comprises administering to the subject an effective amount of an agent that mimics beneficial immune protective effects of Interleukin-18 (IL-18) to promote the differentiation, expansion, and, or function of therapeutic suppressive immune cells (e.g., CD8+ T-cells, CD4+ T-regulatory cells, NK cells, or other IL-18 responsive suppressive immune cell), thereby inhibiting immunopathology in said subject; or administering to the subject an effective amount of a cellular therapeutic comprising cytotoxic cells cultured or conditioned in an agent that mimics beneficial immune protective effects of IL-18, thereby inhibiting immunopathology resulting from pathogenic cells in said subject; and monitoring said subject for improvement in symptoms mediated by pathogenic cells.

In certain embodiments of the method, the T-cells causing immunopathology are autoreactive. Disorders to be treated include, without limitation, multiple sclerosis, rheumatoid arthritis, myasthenia gravis, Autoimmune encephalitis, Addison's disease, celiac disease, type 1 diabetes mellitus, autoimmune thyroiditis, inflammatory bowel disease (IBD, ulcerative colitis, Crohns Disease, Sjogren syndrome, systemic lupus erythematosus, Grave's disease, Crohn's disease, Waldenstrom's macroglobulinemia, hyperviscosity syndrome, monoclonal gammopathy of undetermined origin, POEMS syndrome, myeloma, and macroglobulinemia.

In other embodiments, the disorder is an allergic disorder selected from atopic dermatitis, allergic rhinosinusitis, allergic asthma, and eosinophilic enteritis. In cases where the disorder is a post-transplant disorder, the disorder can be graft versus host disease (GvHD).

The agent used in the method can increase bioactivity of free IL-18 in said subject. In other aspects, the agent reduces binding of IL-18 binding protein (IL-18BP). In some instances, the agent increases bioactivity of free IL-18 and while simultaneously reducing IL-18BP binding. In other embodiments, the agent is a functional IL-18 mimetic, fusion protein, antibody, peptibody, mimic or a decoy resistant IL-18 (DR-18) which, upon delivery to the patient, augments the immunoprotective functions of IL-18. The agent can be administered via any route including a route selected from systemic, intravenous, subcutaneous, topical, aerosolized, or oral administration. In yet another aspect, the differentiation, expansion, and, or function of suppressive CD8+ T cells or the protective IL-18 functions induced by said agent in the patient is monitored.

The IL-18 (DR-18) molecule can comprise an amino acid sequence having at least 85%, at least 90%, at least 91%, at least 92%, at least 93%, at least 94%, at least 95%, at least 96%, at least 97%, at least 98%, at least 99%, or 100% sequence identity with at least one of SEQ ID NOs: 2-63. In certain embodiments, the DR-18 comprises one or more mutations at positions Y1, L5, K8, C38, M51, K53, S55, Q56, P57, G59, M60, C68, E77, Q103, S105, D110, N111, M113, V153, and N155 relative to the wild-type IL-18 amino acid sequence set forth in SEQ ID NO:108. In other embodiments, DR-18 comprises an amino acid sequence selected from SEQ ID NOs: 20, 7, 17-19, 21, 22, 55-57, 62 and 63.

In methods of administration of DR-18 molecule or variants thereof, the amount of the DR-18 administered is from 15 μg/kg to 3000 μg/kg. The agent, agonist or antagonist described herein can be administered via a route selected from systemic, intravenous, or subcutaneous administration. In certain aspects of the method, differentiation, expansion, and, or function of suppressive CD8+ T cells induced by said agent is monitored.

In other embodiments, wherein signaling from said APC engages MHC class II molecules which interact with T cell receptors, said APC being selected from dendritic cells, macrophages, B cells, thymic epithelial cells, vascular endothelial cells, and follicular dendritic cells.

In yet another aspect of the methods provided above, immune protective effects of free IL-18 can include one or more of i) binding of IL-18 to the IL-18R and activation of the IL-18R; ii) regulation of innate and acquired immune responses; iii) induction of one or more T-lymphocyte helper (Th1) responses; iv) enhanced cell-mediated cytotoxicity; v) IFN-γ induction; vi) enhanced production of GM-CSF and IL-2; vii) potentiation of anti-CD3 induced T-cell proliferation; viii) increased Fas-mediated killing by natural killer cells (NK cells) and CD4+ Th1 cells; ix) increased apoptotic death via activation of the Fas-FasL pathway signaling; x) up-regulation of FasL expression; xi) induction of T-lymphocyte helper cell type 2 responses (Th2) in T-cells and NK cells; xii) stimulation of basophils and mast cells to produce Th2 cytokines and histamine; xiii) inhibition of IgE production; and xiv) activation of T cell signaling pathways by APC cells.

In certain embodiments, the antagonist is selected from an inhibitory nucleic acid, an inhibitory antibody, a peptide mimetic, a small molecule, or an IL-18 decoy-to-the-decoy (D2D). In other embodiments, the IL-18 D2D comprises an amino acid sequence having at least 85%, at least 90%, at least 91%, at least 92%, at least 93%, at least 94%, at least 95%, at least 96%, at least 97%, at least 98%, or at least 99% sequence identity with at least one of SEQ ID NOs: 64-97. In certain instances, the DR-18 comprises an amino acid sequence selected from SEQ ID NOs: 64-97.

The methods described herein can further comprise administration of one or more anti-inflammatory agents selected from analgesic agents, disease-modifying anti-rheumatic drugs (DMARDs), non-steroidal anti-inflammatory drugs (NSAIDs). Other agents to be administered include, for example, one or more antiviral, antibiotic, analgesic, corticosteroid, antagonists of inflammatory cytokine, a non-steroidal anti-inflammatory agent, pentoxifylline, thalidomide, azathioprine, cyclophosphamide, cyclosporine, hydroxychloroquine sulfate, methotrexate, leflunomide, minocycline, penicillamine, sulfasalazine, indomethacin; celecoxib; rituxin, rofecoxib; ketorolac; nambumetone; piroxicam; naproxen; oxaprozin; sulindac; ketoprofen; diclofenac; and gold compounds such as oral gold, gold sodium thiomalate, and aurothioglucose.

In certain aspects of the method, the culturing or conditions produce Suppressive CD122+CD8+ T-cells and/or KIR+CD8+ T-cells. In other aspects, the methods employ donor cells haplotype-matched to the subject to be treated. In other aspect, cells are autologous and harvested from the subject to be treated.

Use of a composition comprising an agonist of interleukin 18 (IL-18) or IL-18 receptor (IL-18R), in a method of treating a subject, is also provided, wherein the subject is a human subject having a disease or disorder selected from the group consisting of: autoimmune disease or disorder, allergic disease or disorder, post-transplant disease or disorder, and an inflammatory disease caused by pathogenic CD4+ T cells; and the method treats the subject for the disease or disorder. In certain embodiments, the disease or disorder is multiple sclerosis. The agonist to be administered can be a decoy resistant IL-18 (DR-18) and can comprise an amino acid sequence selected from SEQ ID NOs: 20, 7, 17-19, 21, 22, 55-57, 62 and 63.

Also disclosed is use of a composition comprising an antagonist of interleukin 18 binding protein (IL-18BP), in a method of treating a subject, wherein the subject is a human subject having a disease or disorder selected from the group consisting of: autoimmune disease or disorder, allergic disease or disorder, post-transplant disease or disorder, and an inflammatory disease caused by pathogenic CD4+ T cells; and the method treats the subject for the disease or disorder. In certain approaches, the disease or disorder is multiple sclerosis, and the antagonist is an interleukin 18 decoy-to-the-decoy (IL-18 D2D). In other approaches, the antagonist is an IL-18BP inhibitory antibody that specifically binds IL-18BP.40. The use also includes the antagonist is an IL-18BP inhibitory nucleic acid that prevents translation and/or expression of a nucleic acid encoding IL-18BP.

### BRIEF DESCRIPTION OF THE DRAWINGS

FIGS. 1A-1F: Excess IL-18 protects from Experimental Autoimmune Encephalomyelitis (EAE). FIG. 1A [Prior Art]: Extracted from Shimizu et al.,¹⁷. Two systemic juvenile idiopathic arthritis (s-JIA) subsets: IL-6-dominant subset (red circles), IL-18-dominant subset (black circles). Patients complicated with MAS (green circles). Patients with higher IL-18/IL-6 ratio had significantly less inflammatory arthritis. FIG. 1B: Excess IL-18 is protective against clinical onset features of (EAE). Mean clinical EAE scores of Il18 bp^{-/-} and WT mice immunized with “myelin oligodendrocyte glycoprotein (MOG³⁵⁻⁵⁵) in CFA to induce EAE (n=9). FIG. 1C: Mean clinical scores of Il18tg and WT mice (n=10). FIG. 1D recombinant murine mature IL-18 (1 μg/injection) was administered IP into WT mice thrice on alternating days during induction phase (Days 0, 2, 4) or effector phase (Days 9, 11, 13) (n=8/group). FIG. 1E: Recombinant murine Decoy-Resistant IL-18 (DR-18, Zhou et al.¹⁴, 8 μg/dose, or PBS was administered every 3 subcutaneously. FIG. 1F: IL-18 receptor blocking antibody (clone 9E6, 500 ug/injection) was given IP at the indicated timepoints relative to EAE induction. ****p<0.0001, (FIGS. 1A, 1B, 1C, 1E) repeated measures ANOVA. FIG. 1D Sidak pairwise comparison of indicated groups following two-way ANOVA.

FIGS. 2A-2C: Excess IL-18 promotes CD8, relative to CD4, T-cell activation in mice with Experimental Autoimmune Encephalomyelitis (EAE). FIG. 2A: Representative flow cytometry plots, and % CD8+ T-cells, of spinal cord cells obtained at day 21-25 post induction of EAE as in FIG. 1. Pre-gated on live T-cells. Unpaired T-test. FIG. 2B: Total cells/spinal cord from WT mice treated with DR-18 as in FIG. 1 same condition as in FIG. 1E. FIG. 2C: EAE was induced in mice of the indicated genotypes. 2D2 indicates mice bearing the 2D2 MOG-specific T-cell receptor transgene. *p<0.05, Sidak pairwise comparison of indicated groups following two-way ANOVA.

FIGS. 3A-3C: IL-18 sensing by CD8 T-cells is required for protection from Experimental Autoimmune Encephalomyelitis (EAE). FIG. 3A: EAE was induced as in FIG. 1 in mice of the indicated genotypes (Il18r1^{ΔT}=CD4Cre^{pos}; Il18r1^{flox/flox}. “Il18tg ctrls” in these experiments are CD4Cre^{Neg}; Il18r1^{flox/flox}; Il18tg littermate controls, where the Il18tg derives from Hoshino et al.¹⁸ Repeated measures ANOVA of non-WT groups. FIG. 3B: Proportion of spinal cord immune cells that were CD8+ T-cells day 21-25 post EAE induction. Unpaired t-test of indicated genotypes. FIG. 3C: EAE was induced as in FIG. 1 in mice of the indicated genotypes (Il18r1^{ΔCd8}=ERT2e8iCre; Il18r1^{flox/flox}), with ERT2e8iCre generated in as described in Andrews et al.¹⁹. All mice were administered tamoxifen 1 mg IP on days 4 and 6 post-immunization. Efficient deletion of IL-18R on T-cells, Tregs, or CD8 T-cells was confirmed by flow cytometry.

FIGS. 4A-4B: Excess IL-18 alters CNS CD8 T-cell phenotype in EAE: EAE was induced in mice receiving DR-18 or PBS as in FIG. 1E. FIG. 4A: Activation state (CD44+) and representative flow plots of Day 12 splenic CD8 T-cells stained for Ly49 and (intracellular) Helios directly ex vivo. FIG. 4B: Quantitation of average (n=3-4 per group) Ly49 and Helios expression in CD8 T-cells from quadrants as in FIG. 4A.

FIGS. 5A-5B: IL-18 and autoreactive CD4 T-cells may augment/expand regulatory CD8 T-cells in Experimental Autoimmune Encephalomyelitis (EAE). FIG. 5A: Experimental design for FIG. 5B. EAE was induced in WT mice and splenocytes harvested on day 10 post-immunization. Splenocytes were cultured in media containing IL-2 alone or IL-2+IL-18 for 3 days before magnetic purification and injection of 5e6 cultured CD8 T-cells.

FIGS. 6A-6D: Excess IL-18 protects from EAE without signs of hyperinflammation. (FIG. 6A) Representative images and blinded scoring of spinal cord H&E at peak disease (days 18-21). Two sagittal sections per cord were imaged at 50× with inflammatory foci marked by arrows. Number of foci per section were counted and normalized by section length and averaged. (FIG. 6B) Spleen weight (normalized to body weight) at day 20. (FIG. 6C) RBC, WBC, PLT count at day 20. (FIG. 6D) IFNg serum levels at day 12 and 18 with IFNg-inducible chemokine serum levels at day 12. (A-C) Data pooled from at least 2 experiments. Error bars=SEM. Statistical analysis (FIGS. 6A, 6B) unpaired t-test, (FIGS. 6C, 6D) or unpaired t-tests, p-value with Holm-Sidak correction. Only p<0.05 shown. *p<0.05, **p<0.01,****p<0.0001.

FIGS. 7A-7I 2: Excess IL-18 prevents accumulation of activated CD4 T-cells in the periphery and CNS. (FIG. 7A) Flow cytometric quantification of splenic leukocyte populations in B6 and Il18bpKO mice at day 12. (FIG. 7B) Surface expression of activation markers on splenic CD4Tconv at day 12. (FIG. 7C) Quantitation of individual cytokine production by splenic CD4 Tconv assessed by intracellular cytokine staining (ICS) following PMA/Iono stimulation and MOG35-55 stimulation. Percent of dual-producing IFNg+IL-17A+ cells following MOG35-55 stimulation. (FIG. 7D) Flow cytometric quantification of spinal cord leukocytes on day 18. (FIG. 7E) Quantification of total spinal cord CD4Tconv and individual cytokine production by ICS after MOG-stimulation at days 18-21. (F-I) 2.5×105 CD4 T-cells from naïve 2D2 spleens were transferred to WT and Il18bpKO mice on day −1 followed by EAE induction on day 0. (FIG. 7F) Quantification of 2D2 T-cells at day 12 as a percent and absolute number. (FIG. 7G) UMAP visualization of multiparameter flow cytometric data with expression heatmaps of splenic 2D2 T-cells recovered on day 12 from WT and Il18bpKO mice (see methods for details). (FIG. 7H) Representative flow plots and percentage positive of CD62L and CD49d on total splenic 2D2 T-cells at day 12. MFI of CD49d on effector (CD44+CD62L−) 2D2 T-cells. (FIG. 7I) Quantification of spinal cord-infiltrating CD4 T-cells and 2D2 T-cells at day 18. Dashed line represents the average CD4T-cells detected in an unimmunized WT spinal cord. (FIGS. 7A, 7B, 7C, 7G) Data representative of 3 experiments. (FIGS. 7D, 7E, 7F, 7H, 7I) Data pooled from 2-3 experiments. Error bars=SEM. Statistical analysis: All data analyzed by unpaired t-tests with Holm-Sidak correction if multiple comparisons made. Only p<0.05 shown. ns=not significant, *p<0.05, **P<0.01, *** P<0.001, ****p<0.0001.

FIGS. 8A-8F: IL-18-responsive T-cells mediate protection against EAE. (FIG. 8A) Representative histograms of IL-18R1 expression on NK cells, CD44+CD8 T-cells, and CD44+ CD4 T-cells from day 20 dLN)) FIG. 3A shows the mean EAE clinical score of mice of the indicated genotypes. (FIG. 8B) Flow cytometric quantification of splenic leukocytes at days 20-21. (FIG. 8C) Quantitation of individual cytokine production by splenic CD4 T-cells assessed by ICS following PMA/Iono stimulation. (FIG. 8D) Percent and absolute number of T-cells and (FIG. 8E) FOXP3+ CD4Tregs in spinal cords by flow cytometry. (FIG. 8F) Quantitation of individual cytokine production by spinal cord CD4 T-cells by ICS following PMA/Iono stimulation. All data shown are pooled from 2 independent experiments. Error bars=SEM. Statistical analysis: (FIGS. 8B, 8E) one-way ANOVA with Dunnett's post-test of pairwise comparisons to Il18tg, (FIGS. 8C, 8D, 8F) 2way ANOVA with Dunnett's post-test of pairwise comparisons to Il18tg. Only padj<0.05 is shown. *p<0.05, **p<0.01, ***p<0.001.

FIGS. 9A-9F: IL-18 does not directly inhibit autoreactive CD4 T-cells or protect through FOXP3+CD4Treg. Mean EAE clinical score of mice of the indicated genotypes are shown in FIG. 2C. (FIGS. 9A, 9B) Flow cytometric quantification of splenic and spinal cord T-cells at day 15. (FIG. 9C) Mean EAE clinical score of mice of the indicated genotypes. (FIG. 9D) Representative flow plots of IL-18R1 and FOXP3 expression on day 23 CD4 T-cells. (FIG. 9E) Percent IL-18R1+ on CD44+ CD4Tconv, CD4Treg, and CD44+ CD8T-cells. FIG. 5C shows the mean EAE clinical score of WT (black) or Il18bpKO (color) mice that received different amounts of splenic CD4 T-cells from naïve 2D2 mice on day −1. (FIGS. 9B-9E) Pooled data from 2 experiments. (FIGS. 9D,9E,9F) Data representative of 2 experiments. Error bars=SEM. Statistical analysis: (9C,9F) Kruskal-Wallis of AUC with Dunn's post-test of pairwise comparisons to 2D2; Il18bpKO (See FIG. 2C) or Il18tg (FIG. 9C) or Il18bpKOcontrol (FIG. 5C), (FIG. 9A) unpaired t-tests with Holm-Sidak correction for p-value, (FIG. 9B) unpaired t-tests, (FIG. 9D) one-way ANOVA with Dunnett's post-test of pairwise comparisons to Il18tg. *p<0.05, **p<0.01, ***p<0.001.

FIGS. 10A-10I: Excess IL-18 activates and expands effector CD8 T-cells, while CD8 depletion limits protection: Expression of (FIG. 10A) effector markers and (FIG. 10B) putative suppressive markers on splenic CD8 T-cells from WT and Il18bpKO mice at day 12. (FIG. 10C) IFNg production by day 12 splenic CD8 T-cells following PMA/Ionomycin or MOG35-55 stimulation by ICS. (FIG. 10D) Representative flow plots of day 18 spinal cord T-cells. (FIG. 10E) Quantification of spinal cord CD4 and CD8 T-cells and the corresponding CD4:CD8 ratio. (FIG. 10F) Expression of key effector surface markers on spinal cord CD8 T-cells. (FIG. 10G) Representative flow plots and quantification of dual-expressing CD38+PD-1+CD8 T-cells. (FIG. 10H) Mean clinical score of WT and Il18bpKO mice receiving isotype or CD8-depleting antibodies (200 ug, i.p. every 3 days) following MOG35-55 EAE induction. (FIG. 10I) Representative plots of CD4 versus CD8 expression on splenic T-cells at day 25. (FIGS. 10A, 10B, 10E, 10G, 10H) Data pooled from 2 experiments. (FIGS. 10C, 10F) Data representative of 2-3 experiments. Error bars=SEM. Statistical analysis: (FIGS. 10A-10C, 10E-10G) unpaired t-tests, p-value with Holm-Sidak correction with multiple comparisons, (H) Kruskal-Wallis of AUC with Dunn's post-test of pairwise comparisons of Il18bpKO isotype vs anti-CD8. Only padj<0.05 is shown. *p<0.05, **p<0.01, ****p<0.0001.

FIGS. 11A-11G: Excess IL-18 protects against EAE via IL-18-responsive CD8 T cells. FIG. 3D shows the mean clinical score of mice of the indicated genotypes treated with tamoxifen at days 4 and 6. (FIG. 11A) IL-18R1 expression on activated (CD44+) CD4Tconv and CD8 T-cells. (FIG. 11B) Expression of CD44 on CD4Tconv in dLN. (FIG. 11C) Quantification of T-cell subsets in day 23 spinal cords as percent of CD45, CD4:CD8 ratio, and absolute number. (FIG. 11D) CD8 T-cell phenotype by activation (CD44/CD62L expression) and expression of putative CD8Tsupp markers in dLN. (FIG. 11E) CD8 T-cell expression of putative CD8Tsupp markers and (FIG. 11F) quantification of FOXP3+CD4Treg in day 23 spinal cords. (FIG. 11G) Serum IFNg concentration of the indicated genotypes at day 23. (FIGS. 11A-11G) Data representative of 3 experiments. Error bars=SEM. Statistical analysis: (FIG. 11A-11G) one-way ANOVA with Dunnett's post-test of pairwise comparisons, (FIGS. 11C, 11D, 11F) 2way ANOVA with Dunnett's post-test of pairwise comparisons. All comparisons made to Il18tg control. Only padj<0.05 is shown. *p<0.05, **p<0.01, ***p<0.001, ****p<0.0001.

FIGS. 12A-12I: Synthetic, Decoy-Resistant IL-18 (DR-18) disrupts autoimmune effector activity and protects from EAE immunopathology. (FIG. 12A) Mean EAE clinical score of WT mice treated with PBS or DR18 (2 ug, Subq, q3d) from days 0 to 15. (FIG. 12B) Mean EAE clinical score of WT and E8icre; Il18r1fl/fl mice treated with PBS or DR18 (2 ug, treated every 3 days from 0 to 30). (FIG. 12C) Mean EAE clinical score of WT mice treated with PBS or DR18 at various timepoints indicated below the graph (treatment every 3 days from 0 to 15, colored dot corresponds to DR18 treatment while no dot indicates PBS). (FIGS. 12D-12I) 2.5×10⁵ splenic CD4 T-cells from naïve 2D2 mice were transferred to WT and Il18bpKO mice on day −1. Half of the WT mice were randomized to receive DR-18 or PBS on days 9, 12, & 15. (FIG. 12D) Quantification of transferred splenic 2D2 T-cells, (FIG. 12E) CD49d expression on day 12 splenic 2D2, (FIG. 12F) and CD4Tconv and 2D2 in day 18 spinal cord. (FIG. 12G) Absolute number of splenic FOXP3+ CD4Treg and CD8Teff (CD44+CD62L−) cells over time. (FIG. 12H) Expression of various surface markers and (FIG. 12I) CD38/PD-1 co-expression on splenic CD8 T-cells at day 12 and 18 from WT mice treated with PBS and R18. Data pooled from 2 experiments. Error bars=SEM except for box and whisker plot. Statistical analysis: (FIG. 12A) Mann-Whitney of AUC, (FIGS. 12B,12C) Kruskal-Wallis of AUC with Dunn's post-test of pairwise comparisons to DR18 (FIG. 12B) or PBS control (FIG. 12C), (FIGS. 12D, 12G) unpaired t-tests of WT vs WT+DR18 on days 12 and 18 only with Holm-Sidak correction of p-value, (FIG. 12E) one-way ANOVA with Dunnett's post-test of pairwise comparisons (FIGS. 12H, 12I) unpaired t-tests with Holm-Sidak correction of p-value. Only padj<0.05 is shown. *p<0.05, **p<0.01, ***p<0.001, ****p<0.0001.

### DETAILED DESCRIPTION

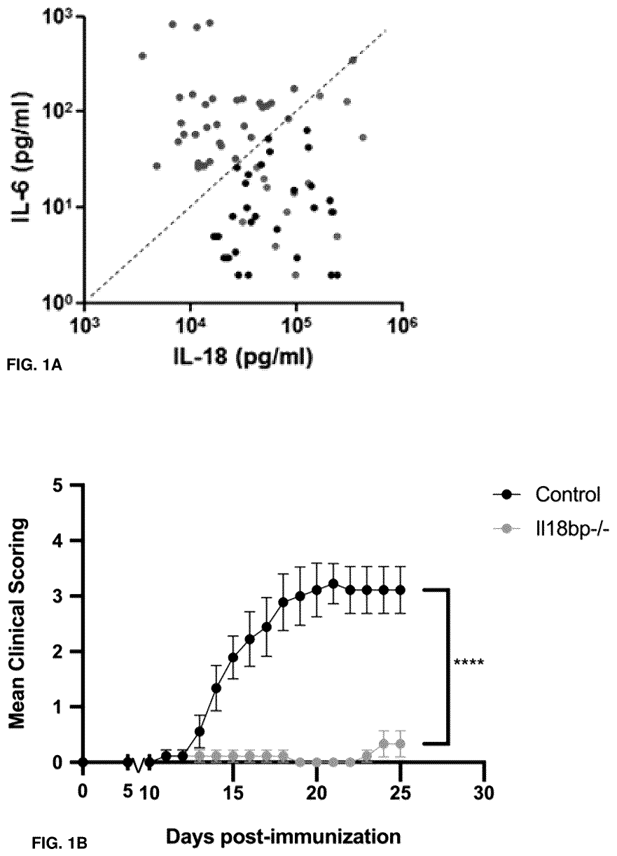

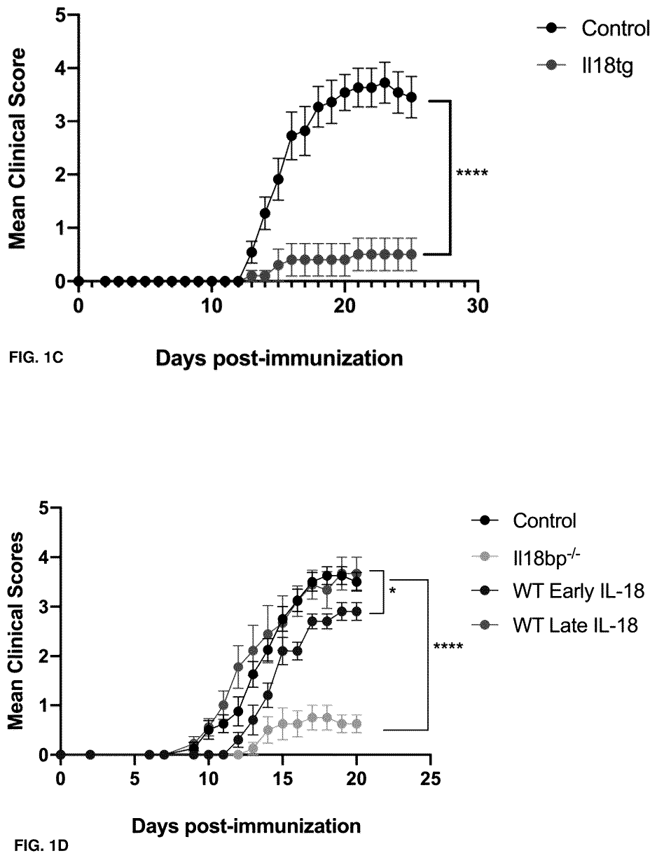

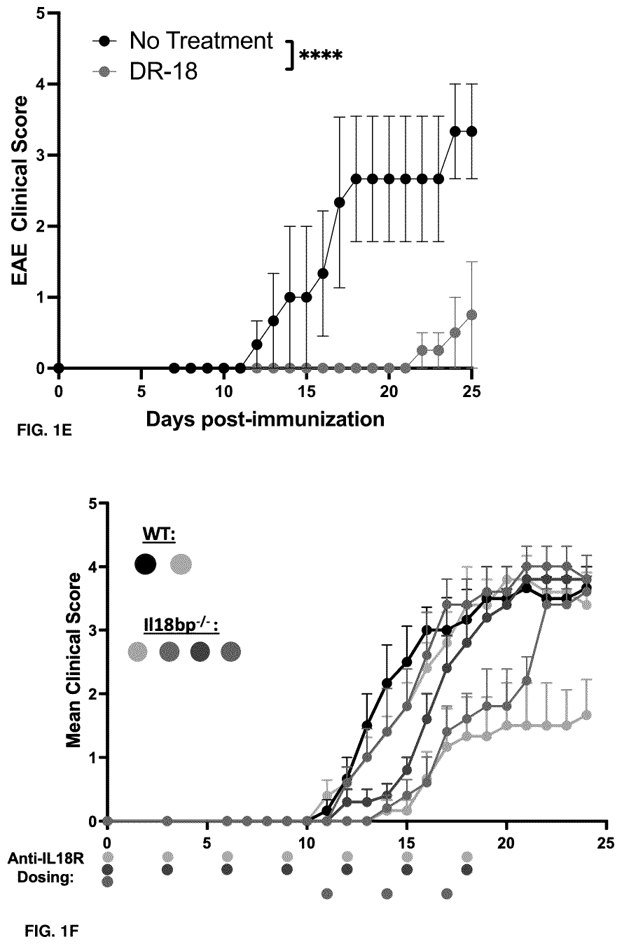

Interleukin-18 (IL-18) is an inflammatory cytokine that has long been associated with the induction of Interferon-gamma. Notably, it has a highly abundant, high-affinity natural antagonist called IL-18 binding protein (IL-18BP). IL-18 levels are modestly elevated in the serum of patients with a wide variety of inflammatory diseases from cancer to lupus, but as noted above, extremely high levels are observed in Systemic Juvenile Idiopathic Arthritis (SJIA) and several monogenic mimics of SJIA. One notable observation in SJIA is that the patients with more IL-18 tend to have more systemic inflammation, but paradoxically LESS inflammatory arthritis. (FIG. 1A). While the systemic inflammation appears to be caused more by innate immune hyperactivation, chronic arthritis is a canonically autoimmune finding associated with diseases like rheumatoid arthritis mediated by autoreactive T-cells and autoantibodies. We follow several patients whose genetic disease results in high levels of serum IL-18, and detectable free IL-18 (unbound by IL-18BP) over their whole lives. Despite chronic inflammation, to date none of these patients has developed autoimmune manifestations. Cancer patients often have extremely high IL-18BP production. This has led to the development of a “decoy-resistant” version of IL-18 (DR-18) that is now in clinical development by Simcha Therapeutics as a cancer immunotherapy (DR-18¹⁴,) and described in clinical trial NCT04787042 titled “Phase 1a and Phase 2 Study for Safety, Preliminary Efficacy, PK and PD of ST-067” (further info available at clinicaltrials(dot)gov/study/NCT04787042)).

IL-18 activity can be augmented in many ways. In vivo, excess IL-18 can be provided via administration of exogenous IL-18 or IL-18 analogues (like DR-18), via use of transgenic expression as in mice engineered to express IL-18 (Il18tg) or Chimeric Antigen Receptor (CAR) T-cells that express IL-18 (CART19-IL18)²⁰, The effectiveness of exogenous recombinant human IL-18 (rhIL-18) has been found to be limited by endogenous production of human IL-18BP. Therefore, in some instances, a DR-18 may be used. IL-18 signaling can be increased by inhibiting the production and/or activity of IL-18BP, for example via use of mice deficient in IL-18BP (Il18 bp−/−), administration of an IL-18BP antagonist, or the like. In these systems, for example via use of mice deficient in IL-18BP (Il18 bp−/−). Excess or unopposed IL-18 has been shown to exacerbate various models of systemic inflammation^{1, 15, 21} CART19-IL18 and DR-18 have been shown to alleviate cancer in murine models by activating T- and NK-cell anti-tumor immune responses¹⁴. Notably, these are NOT models testing the effects of IL-18 on autoimmunity.

It is unclear how excess IL-18 affects antigen specific autoimmunity. A host of correlative work has associated elevated serum IL-18 levels with autoimmune disease and speculated at a role in promoting or exacerbating disease²²⁻²⁴. The experimental autoimmune encephalitis (EAE) murine model is an ideal system to interrogate the role of IL-18 on antigen-specific autoimmunity. EAE is caused by the expansion and Central Nervous System infiltration of T-cells reactive to a peptide present in myelin. Like nearly all activated T-cells, these autoreactive T-cells highly express the IL-18 receptor. Past research on the role of IL-18 in this model has had unclear results but did not focus on IL-18 excess. It is also clear in this model that a certain population of CD8 T-cells is actually protective against the autoimmune manifestations^{12,25}.

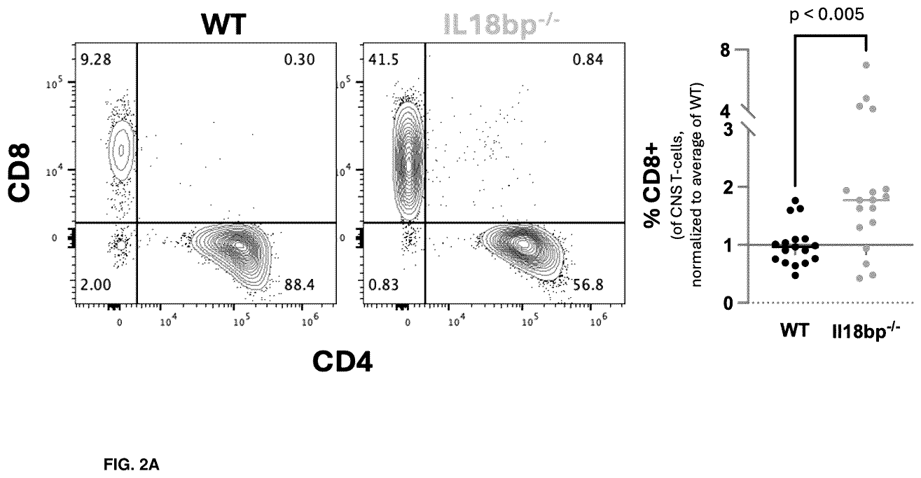

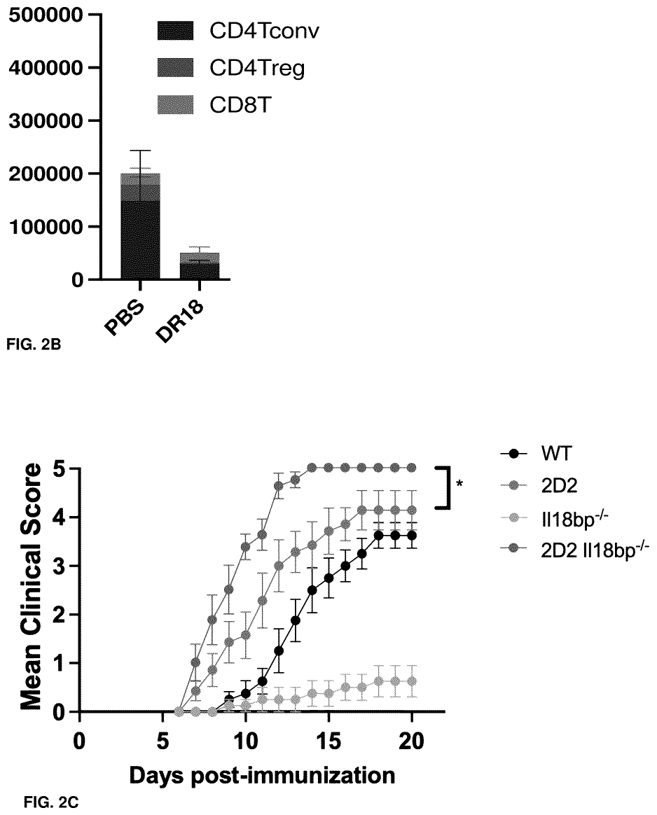

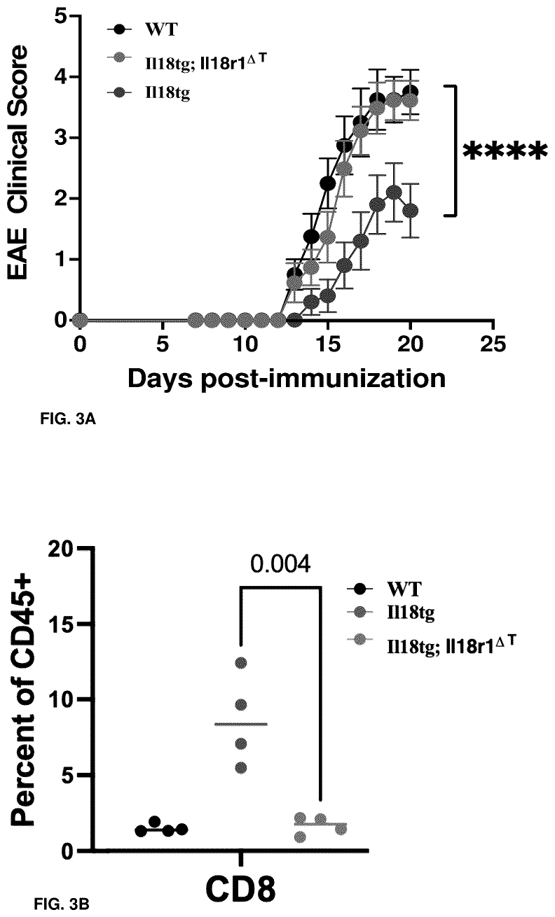

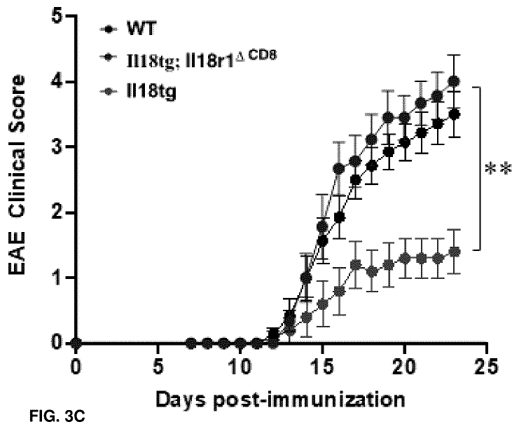

Despite their excess IL-18 activity, we have found that EAE in Il18tg or Il18 bp−/− mice is far less severe than that observed in WT mice (FIGS. 1B, C). Provision of exogenous IL-18 is also partially protective against EAE in WT mice, particularly if given early in the course of disease possibly due to lower levels of IL-18BP early in disease (FIG. 1D). This improvement in disease correlates with a significant increase in the proportion of activated CD8 T-cells in the CNS of Il18 bp−/− mice (FIG. 2). By contrast, we very recently found that unopposed IL-18 became deleterious when baseline proportion of autoreactive CD4 T-cells relative to CD8 T-cells was altered (FIG. 3). This was achieved by breeding mice with transgenic expression of a T-cell receptor specific for the MOG peptide (2D2 mice) with Il18 bp−/− mice. In 2D2 mice, nearly all of the T-cells are CD4 positive and recognize MOG. Il18bp−/−_2D2 mice become sick incredibly quickly, more quickly than 2D2 mice with normal expression of Il18 bp. In this case, these data reveal that unopposed IL-18 can actually promote T-cell mediated autoimmunity.

Thus, the present disclosure provides for the use of IL-18, functional mimetics thereof including DR-18, IL-18 receptor agonists, IL-18BP antagonists and IL-18-treated cellular therapies, to promote the differentiation of cells capable of restricting autoreactive CD4 T-cells, thereby providing therapeutic benefit to the subject. This could be both as an adjunct used in vitro in the development of cellular therapies, or in vivo in affected or at-risk patients. T-cells capable of suppressing pathologic CD4 T-cell responses have been observed not just in autoimmunity, but in graft-versus-host disease (GvHD). Thus, IL-18 can be used to advantage in promoting the expansion and activity of protective T-cells in CD4 T-cell mediated GvHD (Zheng J. et al., Human CD8+ regulatory T cells inhibit GVHD and preserve general immunity in humanized mice. Sci Transl Med. 2013 Jan. 16; 5(168):168ra9; Agle K. et al., Bim regulates the survival and suppressive capability of CD8+ FOXP3+ regulatory T cells during murine GVHD. Blood. 2018 Jul. 26; 132(4):435-447. doi: 10.1182/blood-2017-09-807156. Epub 2018 May 16; Dai Z. et al., Natural CD8+CD122+ T cells are more potent in suppression of allograft rejection than CD4+CD25+ regulatory T cells. Am J Transplant. 2014 January; 14(1):39-48. doi: 10.1111/ajt.12515. Epub 2013 Nov. 12).

### Definitions

The phrase “Myelin-associated autoimmune diseases” refers to central nervous system (CNS) demyelinating autoimmune diseases are autoimmune diseases which primarily affect the central nervous system. Examples include, without limitation, diffuse cerebral sclerosis of Schilder, acute disseminated encephalomyelitis, acute hemorrhagic leukoencephalitis, multiple sclerosis, transverse myelitis and neuromyelitis optica.

“Experimental autoimmune encephalomyelitis (EAE)”, another myelin associated autoimmune disease is a widely used animal model that is characterized by an inflammatory attack against myelin components of the CNS. EAE is primarily mediated by CD4 T cells, which are directed against defined epitopes of CNS myelin. These self-reactive T cells are normal constituents of the peripheral T cell repertoire and can be activated in vivo by subcutaneous. immunization with myelin antigens. The central role of CD4 T cells in EAE is demonstrated by their ability to transfer disease when injected into unchallenged hosts.

As used herein, “interleukin-18 (IL-18)”, also known as “interferon-gamma inducing factor” is a cytokine, which is produced by a variety of myeloid cells, activated macrophages (including Kupffer cells), and barrier epithelial cells of the intestine, skin, and lung. IL-18 binds to IL-18 receptor alpha (IL-18Rα) and IL-18 receptor beta (IL-18Rβ) to form the IL-18 receptor complex, which formation and induces IL-18 signaling. References herein to “IL-18R” will refer to the IL-18 receptor generally either as an individual component thereof (e.g., as in IL-18Rα, such as in the context of IL-18 binding to IL-18Rα) or collectively as the receptor complex (e.g., as in formation of the complex through binding between IL-18, IL-18Rα, and IL-18Rβ to form the signaling complex), which will be readily understood by the relevant skilled artisan. Recent murine evidence suggests IL-18 signaling in some instances may be mediated in part through a Na—Cl symporter (NCC, encoded by Slc12a3)(26-28), and the scope of this disclosure is not limited to the effects of IL-18 mediated solely through IL-18Rα, IL-1Rβ, and/or NCC. requires a IL-18 signaling is typically dependent on the adaptor MyD88 via canonical NF-kB and MAPK pathways. Defects (e.g. knock-out) of the IL-18 receptor (IL-18Rα and/or IL-18Rβ) or IL-18 lead to impaired natural killer (NK) cells activity and T-cell responses. Apart from its physiological role, excess IL-18 may also induce severe inflammatory disorders. For the purpose of early diagnosis of such disorders it therefore may be useful to quantify the levels of free IL-18 in body fluids of a subject, expected to have such a disorder. For example, recombinant IL-18 is described in U.S. patent application Ser. No. 16/123,063 to Aaron Ring, which is incorporated herein by reference.

As used herein, the term “activity” refers to a biological activity.

As used herein, the term “pharmacological activity” refers to the inherent physical properties of a peptide, polypeptide or small molecule. These properties include but are not limited to half-life, solubility, and stability and other pharmacokinetic properties.

The terms “high,” “higher,” “increases,” “elevates,” or “elevation” refer to increases above basal levels, e.g., as compared to a control or reference level. The terms “low,” “lower,” “reduces,” or “reduction” refer to decreases below basal levels, e.g., as compared to a control or reference level.

The term “modulate” as used herein refers to the ability of a compound to change an activity in some measurable way as compared to an appropriate control. As a result of the presence of compounds in the assays, activities can increase or decrease as compared to controls in the absence of these compounds. In some instances, an increase in activity is at least 25%, at least 50%, at least 100% compared to the level of activity in the absence of the compound. Similarly, a decrease in activity is, in some instances, at least 25%, at least 50%, at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

The phrase “acceptable carrier” refers to a material that is not biologically or otherwise undesirable, i.e., the material can be administered to a subject along with the selected compound without causing any undesirable biological effects or interacting in a deleterious manner with any of the other components of the composition in which it is contained. Other compounds will be administered according to standard procedures used by those skilled in the art.

As used herein, “subject” includes vertebrates, more specifically a mammal (e.g., a human, horse, pig, rabbit, dog, sheep, goat, non-human primate, cow, cat, guinea pig or rodent). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium, a yeast, a mammal, etc. A cell can be a eukaryotic cell or prokaryotic cell. In some instances, a cell is a human cell. In some instances, a cell may be obtained from a subject, treated, cultured, and/or modified in some way, and then administered to the same subject in an autologous manner. In some instances, a cell may be obtained from a donor, treated, cultured, and/or modified in some way, and then administered to a different subject in an allogeneic manner. Accordingly, cell therapies may be “autologous” or “allogeneic”, as desired.

By the term “effective amount” of a compound as provided herein is meant a nontoxic but sufficient amount of the compound to provide the desired result.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

An “antagonist” (also sometimes referred to herein as an “inhibitor”) as defined herein, is a molecule that decreases, or prevents, a known activity. An antagonist may directly inhibit a signaling pathway, e.g., by partially or completely blocks blocking the binding of two cognates or members of a specific binding pairs, thereby inhibiting the downstream biological effects of the cognates or binding pair interaction. For example, an antagonist may block the binding of a ligand to its receptor, which in turn reduces and/or prevents intracellular signaling via activating that receptor, which in turn reduces or prevents the downstream biological effects of activating that receptor, such as but not limited to, cell activation, proliferation, differentiation, cytokine release, up-regulation of genes, cell-surface expression of proteins, and the like. Activating or activation of a receptor is defined herein as the engagement of one or more intracellular signaling path way(s) and the transduction of intracellular signaling (i.e., signal transduction) in response to a molecule binding to a membrane-bound receptor, such as but not limited to, a receptor:ligand interaction.

In some instances, an antagonist may decrease or prevent the activity of an inhibitory molecule, such as a ligand binding protein that binds to and inhibits the function of a ligand (also sometimes referred to as a “decoy” or “decoy receptor”). In such instances, the antagonist may inhibit the inhibitor, thereby increasing signal transduction associated with the ligand. For example, useful antagonists include IL-18BP antagonists, such as an anti-IL-18BP antibody or a IL-18 mimic that preferentially binds IL-18BP. Useful IL-18BP antagonists bind IL-18BP and prevent it from binding to and inhibiting IL-18 (i.e., preventing IL-18BP inhibition of IL-18). An antagonist may also be a molecule that decreases or prevents the expression of a target, thereby decreasing or preventing activity associated with the target, e.g., any signal transduction or inhibitory function associated with the target. For example, an antagonist may be a small molecule, such as e.g., an interfering nucleic acid (e.g., a small interfering RNA, a short-hairpin RNA, an antisense DNA or RNA, etc.) that decreases or prevents the expression of the target of the interfering nucleic acid. In some instances, an antagonist that decreases or prevents the expression of a ligand binding protein may be employed, thereby inhibiting the inhibitory function of the ligand binding protein. For example, an antagonist that decreases or prevents the expression of IL-18BP may be employed, thereby inhibiting IL-18BP mediated inhibition of IL-18 signaling.

“Signal transduction, as used herein, is the relaying of a signal by conversion from one physical or chemical form to another. In cell biology, this is the process by which a cell converts an extracellular signal into a response. As described above, useful antagonists are those that inhibit IL-18BP activity.

An “agonist” as defined herein, is a molecule that increases a known activity. Agonists presented herein include antibodies, fusion proteins, mimetics, peptibodies, small molecules, and other recombinant molecules. Useful agonists include those having binding affinity for one or more of the following: IL-18, IL-18R (including IL-18Rα and/or IL-18Rβ) which can increase the immune protective functions thereof. One such category of molecules, are DR-18's, which bind to, and allow formation of the IL-18 receptor complex, but are not bound by IL-18BP, and are thus not subject to IL-18BP inhibition of IL-18 signaling.

A “peptibody” refers to molecules comprising an Fc domain and at least one peptide. Such peptibodies may be multimers or dimers or fragments thereof, and they may be derivatized. Peptibodies are described in greater detail in WO 99/25044 and WO 00/24782, which are incorporated herein by reference in their entirety. The peptide may be from the amino acid sequence of IL-18, IL-18 receptor or (IL-18R), IL-18 binding protein (IL 18BP) for inhibition or reduction of IL-18BP function or activity, and can be a substance, compound or composition designed to mimic the activity of the native molecule and augment the desired signaling pathway.

A “peptide”, as used herein refers to molecules of 1 to 40 amino acids. Alternative embodiments comprise molecules of 5 to 20 amino acids. Exemplary peptides may comprise portions of the extracellular domain of naturally occurring molecules, comprise randomized sequences, or be rationally designed. In some embodiments, useful peptides include e.g., portions of an IL-18 sequence, or derivatives or modified versions of one or more portions of an IL-18 sequence, which inhibits the binding of IL-18 to IL-18BP to alter activity.

A “peptidomimetic” is a peptide analog that displays more favorable pharmacological properties than their prototype native peptides, such as a) metabolic stability, b) improved bioavailability, c) high receptor affinity and receptor selectivity, and d) minimal side effects. Designing peptidomimetics and methods of producing the same are known in the art (see for example, U.S. Pat. Nos. 6,407,059 and 6,420,118). Peptidomimetics may be derived from the binding site of the extra cellular domain of IL-18, IL-18 receptor (IL-18R), IL-18 binding protein (IL-18BP). In alternative embodiments, a peptidomimetic comprises non peptide compounds having the same three-dimensional structure as peptides derived from IL-18, IL-18 receptor (IL-18R), IL-18 binding protein (IL 18BP), and, or DR-18, or compounds in which part of a peptide from the molecules listed above is replaced by a non-peptide moiety having the same three-dimensional structure.

Embodiments of the present disclosure provide compositions and methods for inhibiting immunopathology resulting from pathogenic immune cell activity, e.g., from T cells (CD4+, CD8+, NKT, gamma-delta T), Natural Killer (NK) cells, B-cells, and Antigen presenting cells (APCs). In some embodiments, the agent can be a functional IL-18 mimetic, an agonist, and/or a decoy resistant IL-18 (DR-18).

IL-18:IL-18R biological activity, is defined herein as including, but is not limited to, binding of IL-18 to the IL-18R and activation of the IL-18R; regulation of innate and acquired immune responses; proinflammatory effects; induction of T-lymphocyte helper cell type 1 responses (Th1); enhanced cell-mediated cytotoxicity; IFN-γ induction; enhanced production of GM-CSF and IL-2; potentiation of anti-CD3 induced T-cell proliferation; increased Fas-mediated killing by natural killer cells (NK cells) and CD4+ Th1 cells; increased apoptotic death via the Fas-FasL pathway; up-regulation of FasL expression; induction of T-lymphocyte helper cell type 2 responses (Th2) in T-cells and NK cells; stimulation of basophils and mast cells to produce Th2 cytokines and histamine; induction of IgE production. Such IL-18 agonists include, but are not limited to: decoy resistant IL-18 (DR-18) antibodies directed against IL-18 that specifically bind IL-18 and partially or completely enhance binding of IL-18 to IL-18R: antibodies, fusion proteins and/or peptibodies directed against IL-18R that specifically bind IL-18R and enhance receptor binding of IL-18 without themselves transducing a signal via IL-18R; small molecules that bind IL-18 or IL-18R that increase the interaction thereof, such as IL-18 and/or IL-18R peptidomimetics.

As used herein, when reference is made to making IL-18 agonists based on IL-18, or IL-18 receptor agonists, or IL-18 BP antagonists, it is understood that the terms IL-18 Binding Protein antagonists, and agonists of IL-18, DR-18, and IL-18 receptor also encompass fragments, variants, muteins, derivatives and fusion proteins thereof, as described in detail below. The isolation, cloning, preparation and characterization of human IL-18 receptor (referred to interchangeably as IL-18R or huIL-18R) are described in U.S. Pat. Nos. 6,087,116 and 6,589,764 (PCT Publication WO99/37772), which are incorporated herein by reference in their entirety.

The phrase “Antigen Presenting Cell (APC)” refers to cells that can process a protein antigen, break it into peptides, and present it in conjunction with class II MHC molecules on the cell surface where it may interact with appropriate T cell receptors. “Professional” APCs include dendritic cells, macrophages, and B cells, whereas “nonprofessional” are APCs that function in antigen presentation for only brief periods include thymic epithelial cells and vascular endothelial cells. Dendritic cells, macrophages, and B cells are the principal antigen-presenting cells for T cells, whereas follicular dendritic cells are the main antigen-presenting cells for B cells. The immune system contains three types of antigen-presenting cells, i.e., macrophages, dendritic cells, and B cells.

CD8+ regulatory T cells that are essential for maintenance of self-tolerance and prevention of autoimmune disease. In some aspects, a method for treating an autoimmune disease is provided. The method involves administering to a subject in need of such treatment an IL-18 receptor (IL-18R) agonist in an amount effective to ameliorate a symptom of the autoimmune disease.

Aspects of the disclosure describe methods of treating autoimmune diseases by isolating T cells from the subject in need of such treatment and culturing them in medium enriched with IL-18 or a mimetic or agonist thereof.

In some embodiments, the isolated cells are grown until the population of the specialized regulatory/suppressor T cells increase in number by at least 5%, 10%, at least 20%, at least 30%, at least 50%, at least 75%, at least 90%, at least 100%, or at least 200%. In some embodiments, after culturing isolated T cells, specialized regulatory T cells may be further enriched by isolating specific populations including but not limited to CD4+CD25+ (with or without expression of CD127 or CD45RA), T regulatory cells expressing CD8+CD122+ (with or without expression of Kir or Helios) and T suppressor cells. In some embodiments, the specialized regulatory/suppressor T cells are administered to the subject by intravenous injection. The specialized regulatory/suppressor T cells are separated from the culture medium before administration to the subject. The specialized regulatory/suppressor T cells are administered to the subject in an amount effective to ameliorate a symptom of the autoimmune disease.

In some embodiments, the specialized regulatory/suppressor T cells are administered in combination with an autoimmune drug. Non-limiting examples of such drugs include methotrexate, cyclophosphamide, Imuran (azathioprine), cyclosporin, and steroid compounds such as prednisone and methylprednisolone.

SEQ ID NO: 1
(UniProt O95998)
MTMRHNWTPDLSPLWVLLLCAHVVTLLVRATPVSQTTTAATASVRSTKDP
CPSQPPVFPAAKQCPALEVTWPEVEVPLNGTLSLSCVACSRFPNFSILYW
LGNGSFIEHLPGRLWEGSTSRERGSTGTQLCKALVLEQLTPALHSTNFSC
VLVDPEQVVQRHVVLAQLWAGLRATLPPTQEALPSSHSSPQQQG
SEQ ID NO: 98
(UniProt Q14116)
MAAEPVEDNCINFVAMKFIDNTLYFIAEDDENLESDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNED

As mentioned above, preferred antagonists comprise those that target IL-18BP. PCT Publication WO99/09063 (incorporated by reference herein) describes the IL-18BP, including useful soluble fragments thereof. One embodiment of a human IL-18BP is the “a” isoform having the polynucleotide sequence described herein as SEQ ID NO:99 and the corresponding amino acid sequence described herein as SEQ ID NO: 100.

SEQ ID NO: 99
gtcgacgg-a cccccgggaa agatttaata cgactcacta tagggcggga cagaartgat ctgtgagaga ctcatctagt
tcatacccta ggtgacccrg ggggtggcat gggggragat tagagatccc agtctggtat cctctggaga gtaggagtcc
caggagctga aggtttctgg ccactgaact ttggctaaag cagaggtgtc acagctgctc aagattccct ggttaaaaag
tgaaagtgaa atagagggtc ggggcagtgc tttcccagaa ggattgctcg gcatcctgcc cttcccagaa gcagctctgg
tgctgaagag agcactgcct ccctgtgtga ctgggtgagt ccarattctc rctttgggtc tcaattttgc cttccctaat gaaggggtaa
gattggacta ggtaagcatc rtacaaccat ttgtggtcat gagagctggg grggggaagg attgtcacrt gaccccccca
gctctgtttc taagtgctga aagagctcca ggctatgcta cgggaggaga agccagctac tgaggaaaag ccagctactg
agaaaaagcg ggagtggttt accattctcc tcccccacct ttcaccagag aagaggacgt tgtcacacat aaagagccag
gctcaccagc tcctgacgca rgcatcatga ccatgagaca caactggaca ccaggtaggc cttggggcta cgcatgggca
ggcggggtag ggtgaggtct atgaacagaa tggagcaarg ggctaacccg gagccttcac rccaaggcaa accacccagc
gcacctggrg crgrtgcttt aagaacctgg gcagatarrg ragctctggc tccagtctaa agcttctcrg tacrctgtrc
aataaagggc taaggggrgg gtgctgaggg gtccctcttc ccgctctgat tccctggcta gaacccagac atcrcrgggc
rggagrtaca tccttacccg ggcagcccac tctgtctcca gagccgctga cctgtaactg tcctttcctc agacctcagc
cctttgtggg tcctgctcct gtgtgcccac gtcgtcactc tcctggtcag agccacacct gtctcgcaga ccaccacagc
tgccactgcc tcagttagaa gcacaaagga cccctgcccc tcccagcccc cagtgttccc agcagctaag cagtgtccag
cartggaagt gacctggcca gaggtggaag tgccactgag raagaagcac agrggtggag ggtgggcrat gggcacagag
gttcccaggg rcgggttgac tcctgagcgc cagtcccctt crgcccatgt accaccagct gagccagctg ggctgagcac
gcaccattct ccctccccaa cccagtgtca tgggrgcagg crrggcgcag crcccaagat gctccctatc aaataggaca
gagaactcaa gacataagta atggtcacag gacctcccag agccttggtt gcagtggacc ccaaggccag cccctccacc
cagagcct c tggcctctgg ccatctcaga ggagcagcag ccatccagca ctgcctctgt cacctgggcr cccaagtcac
cgaggctggg cactagaaaa ggtcatcctg aggagacagg ttcagaagag gartcatcac gtgaaccaag gaccattcct
cacattcccc gtgtttaggg ctagggcctc tcggagacaa ctgcacrtct gtaacggacg rtcccaccta ggtggtgtgc
agagcagttc tctaggttcc agatgcatgg ggactggggg gagctggcag agagggcaca gcagagcagg graggggaag
ggcctgctct tctgaagagc raactgctgc crgtgtccct agatggaacg ctgagcttat cctgtgtggc ctgcagccgc
ttccccaact tcagcatcct ctactggcrg ggcaatggtt ccttcattga gcacctccca ggccgactgt gggaggggag
caccaggtga gggtcgcagc agccaggtgg gtgggaacga agcctrctgc ggccrtctca rgacctrtcc ttcccttccg
ctccagccgg gaacgtggga gcacaggtac gcagctgtgc aaggccttgg tgctggagca gctgacccct gccctgcaca
gcaccaactt ctcctgtgtg ctcgtggacc ctgaacaggt tgtccagcgt cacgtcgrcc tggcccagct ctgggtgagg
agcccaagga gaggcctcca ggaacaggag gagctctgct tccatatgtg gggaggaaag ggtgggctct gccagagcag
cctgtgaact aatgcccagc attcctcaag gtcagccaga caaaaaggaa cttaggtctt gggcagagga ggtgtagcct
ggggcaaagt gatgagatgt ccctcctttc cttggcctga tccttgtctg ccttcacttc cctaggctgg gctgagggca
accttgcccc ccacccaaga agccctgccc tccagccaca gcagtccaca gcagcagggt taagactcag cacagggcca
gcagcagcac aaccttgacc agagcttggg tcctacctgt ctacctggag tgaacagtcc ctgactgcct gtaggctgcg
tggatgcgca acacaccccc tccttctctg ctttgggtcc cttctctcac caaattcaaa ctccattccc acctacctag aaaatcacag
cctccttata atgcctcctc ctcctgccat tctctctcca cctatccatt agccttccta acgtcctact cctcacactg ctctactgct
cagaaaccac caagactgtt gatgccttag ccttgcactc cagggcccta cctgcatttc ccacatgact ttctggaagc
ctcccaacta ttcttgcttt tcccagacag ctcccactcc catgtctctg ctcatttagt cccgtcttcc tcaccgcccc agcaggggaa
cgctcaagcc tggttgaaat gctgcctctt cagtgaagtc arcctctttc agctctggcc gcattctgca gacttcctat cttcgtgctg
tatgtttttt ttttccccct tcactctaat ggactgttcc agggaaggga tgggggcagc agctgcttcg gatccacact gtatctgtgt
catccccaca tgggtccrca taaaggatta ttcaatggag gcatcctgac atctgttcat ttaggcttca gttccactcc caggaacttt
gcctgtccca cgagggagta tgggagagat ggactgccac acagaagcrg aagacaacac ctgcttcagg ggaacacagg
cgcttgaaaa agaaaagaga gaacagccca taatgctccc cgggagcaga ggccactaat ggagagtggg aagagcctgg
aaagatgtgg cctcaggaaa agggatgaga gaaaggaggt ggtatggaag actcagcagg aacaaggtag gcttcaaaga
gcctatattc ctctttttcc cacaccgatc aagtcaactc agtactcacg ggagaaaaat agactttatt tacaagtaat aacatttaga
aaagatccar ccccggccct taaaaacctt cccatcactc caaatcccac cccagtgcaa gtctggggaa ggtagggtgt
gagctgctgc tgaaggctgt cccccaaccc cactcctgag acacagggcc catccgtcct gggaaagagc atcctctggc
aggrgctccc accaggtcag acccagtcct ggacttcaag agtgagggcc cctgctgggc ccagccacca ggacagcagg
aaccagggcc tactcctctt atggtccctt ctagatccag aggctaagag gaagactggc caggcccaag gacccagcca
tcaaaaccag cctcaaatct ggttgtgatg gagaagtgac tttgctttaa gaaaaaagga ggcaaggtag ggagagcgcc
cacactgtcc atgctccagg ccccctgggc cagctccgag aaggcgccag tgaaggacca gggaccaggc cagggtgcgg
gcaggcatca ctgtctctag gggttrggct actgttggcc rgggagctga gagaaggcac tgagagggac agtaggcgga
ggaccaggrg acggcagcar cggggacaca ggtggggcca ctcactggra ctggcccttt agtgctttgc ctgaaagaga
cacagtcaca tggccagatg agaacttgcg atactagccr gcacccactg gctgggaaga rctcttcctg ctcccacgcc
cctgtctgga tcccctccct tgtgagcccc agggttatca gtrgctggct gtgcctgagc agctctgggt gctctccatg
agaarggggc catctgtctt ctctccttgg agaggagcta ccaggacagg gacacctctt accccacacc ctccagcagc
ctggcgtggc cccatcttgg atgctacttg gtggggcggt ctggggggtg cccatgctct catcgggttt ccctccccca
tcctgccagt gcctctacct tgcccttggc tcgaggggtg gcaccaatgg cggcagcagt ggcggcgctg gctgtggtgg
rggcaatgcg cggagaacgg cgggttccac tgcgagtgtt gggggaagcc ttggacaggg ccttctttga ggctccccgc
cgcagaaggc tgttccctag cttcttgggt gtgttgagga rgcrgaaggc catcgactgg cgccggtcag cctgcaagga
agggctgtca gaccgggaga cccaargctg ccttcccagg ccagcgtgct gtgccacgct gtaccagcaa ggtcccgcca
gggcgtcgct tcatccccct tcagccccag cctcacctgt rragtagaag ctggagctgc rttcrtctgg gcctcagtag
tgctctgttt gcgcccttca tgtcggtctc ggggagtcat ggggcgtggg aaacagctgg tggccttctt agactatgga
gaagaggaca gttaggcaga cagragcaag aggagtcaca tctgaagcca ggtgtcttgt cctctcagag ctgagtggac
cttgraagtc aacgtgcaac ctgctcccct tcccaactct gggccagatc cttcccttcc caacagttcc catccatggg
tcaggccctt ggagagaggg aaagagaggg ggaagtgagg gaaggagaga gaaggctccc tttagtcctt ggtgagctgg
gcctgacctg agcacagtgc tggagraaca cccaggagcc accgcgccta cctcaggagt tccagggccc tggtggggct
ctagggagac ccgtttgcgc tgctgccggg tggtgatgcc agtgccctcg gctatctgga ttggctgcat gctggctcgg
cgcagggtct cttgggggtc tccagttttc atctcctcat ctgtgatggt gcccaggctc agggaaggct gcatgggtgg
aagaggtggt cagtggacca tagctgtatg gagatggagg aggacctggg gctgttccag aactctacac tcgcccgaca
cttatggtcg ggacccttcc tgcctacgag gtagaaagac acaagcctcc tttcctgttc tgctttctac ctaagccctg
ggcaaatggc acaagcagtg cagtcctgac cagattcctc tctgagctcc tgcctacccc cagggacttc acccctgagt
gccctccagc tgtctgttcc acctggaaca tgagaaggtc accccttccc ctcttcggcc agtcagtgat ccagggccct
agtgctcagg ctagarcagc aggtgggart ccaaggaagg gcagggargg gaggccctgc acagtgaccc caggcctcac
cctggactcc agggatagca ggtcttcaga tgtggggggc acactcgatt gcgctgctgc agctctgcaa tgcggttcca
gtcarccagc tgctcaggct catcctggca agtgcccatg tagaagctgt tccttcctgt ggaaggcagg gaagtgggaa
caaatgagcc tggagtcggc aggtcacctc ctggccctgg catcttgcca gcctttgctg ccacctaccc cataaacttg
aagcccggca caccagtctg attcagtgcc gcaggtgcag gagtacggca cacagactat ttctatccta ggggcttgct
caccaccttc tccctggaga gggcagaaga ggtcacacgc agagactgct actacatctt attcacctgc caaggcttgg
tggccaacac ccagaggaac aaattaagga ccgggaatta attcccaggg gctccctggt gcccaaagga caagagcttc
caagaagagt ctggccagcc tggcctttcc agcagcccat caccgcctga gaagggcarg gaggactccc cacagctaag
rgrcacaart gtgctgggaa tcccgggccc ttaactctgg ctaagagtgc ccccaacaca gccagcccct agatgggcag
gtaaggaagg ccctgaggct gcaggaagga ggggcaggtg gagctggatg gtagcaagga ggccagcctt ggatttttaa
aaagctttcc tcttttccct gtgccacgat ccaccttcca gtctaatttt ggggtatagt aagtccctgt agtcccctca cctggagggg
ccccactgga caccccggcc tgggaacgac gagcagaact gcgagtggtg gggcggtagc caggcaagct gagcagggct
gagttgccat aatcgggaga acccaggcga gctagagact gagtagagga ggtggcrcgc aggctagcct gggaagcagg
agcagaccgc gtgcrgtaga acgatgagtt ggcgctgtct ggctcttcca catctagctt ctggaagaca gagtgaarcr
gttgcagtgt acagrccctg gcactgtaca gaagcttccc attcccttcc gaagcccrca gatcccacgg cacatccatg
tattcccaac rgcrttgcaa aggrccrraa agrgtgrgrc tgcaagaaar gggccrrgtc gacagaagcc ctcacaaggt
ggtgctgarg ttgtcaagac tcttctacgc atttttttca tggagtctat rcaraatgct ttgaggtagg gaatgcagag tgtttatcgg
cccatrttgg agatgaagtg caaagaaata aagtgactag ccccaaarca cactgctagg aagtatcaga gctggggcra
ggccccargt ctcctgacta gtcaggctca tcccacagcc tctgctgtcc ctcagtccaa acttccaggg cccttaccat
gttccagaac ttcccccaac ttcttggtag cagggggcac cctaaacaca caggtccccc ctgctgtacc aggggccccc
tctcccctcc tcccaaacct ccccttcaag atgtggaaac aaaggcaagg gcctgcagcc tgtcaggcag tccactgggc
agcaacaatg cctctcagct gcatggggca tgctgggagg cacaggatgg gctgcagctr cgccacgttc tctcccttca
ccctgcacag gctcagtgct acgcatggag agaatgcrag ccttagtcag gaggcaggga tctaatccra gccctgcctt
tttcttcaga agtgccctta accaagtcac tgcccttttt aagaccrctc agctttccca ctgtaacatg gactggctgc tcatccctcc
ctgctccrga ctgagrgccc ag
SEQ ID NO: 100
Thr Pro Val Ser Gin Thr Thr Thr Ala Ala Thr Ala Ser Val Arg Ser Thr Lys Asp Pro Cys Pro Ser
Gin Pro Pro Val Phe Pro Ala Ala Lys Gin Cys Pro Ala Leu Glu Val Thr

Of course, other IL-18BP isoforms that are antagonistic to IL-18 binding to IL-18R may be used. Such as the b, c and d isoforms. The polynucleotide and amino acid sequences for the b, c and d isoforms are known in the art and readily available (see for example, Kim, S.-H. et al., PNAS 97:3 1190-1195 (2000)).

A particularly useful form of the IL-18 binding protein, found in U.S. Patent Publication No. 2009/0297517 (incorporated herein by reference), is a fusion with an Fc domain of an antibody. The amino acid sequence of such a fusion protein, termed IL-18BP-Fc herein, is described herein as SEQ ID NO:101.

SEQ ID NO: 101
Met Arg His Asn Trp Thr Pro Asp Leu Ser Pro Leu
Trp Val Leu Leu Leu Cys Ala His Val Val Thr Leu
Leu Val Arg Ala Thr Pro Val Ser Gln Thr Thr Thr
Ala Ala Thr Ala Ser Val Arg Ser Thr Lys Asp Pro
Cys Pro Ser Gln Pro Pro Val Phe Pro Ala Ala Lys
Gln Cys Pro Ala Leu Glu Val Thr Trp Pro Glu Val
Glu Val Pro Leu Asn Gly Thr Leu Ser Leu Ser Cys
Val Ala Cys Ser Arg Phe Pro Asn Phe Ser Ile Leu
Tyr Trp Leu Gly Asn Gly Ser Phe Ile Glu His Leu
Pro Gly Arg Leu Trp Glu Gly Ser Thr Ser Arg Glu
Arg Gly Ser Thr Gly Thr Gln Leu Cys Lys Ala Leu
Val Leu Glu Gln Leu Thr Pro Ala Leu His Ser Thr
Asn Phe Ser Cys Val Leu Val Asp Pro Glu Gln Val
Val Gln Arg His Val Val Leu Ala Gln Leu Trp Ala
Gly Leu Arg Ala Thr Leu Pro Pro Thr Gln Glu Ala
Leu Pro Ser Ser His Ser Ser Pro Gln Gln Gln Gly
Arg Ser Cys Asp Lys Thr His Thr Cys Pro Pro Cys
Pro Ala Pro Glu Ala Glu Gly Ala Pro Ser Val Phe
Leu Phe Pro Pro Lys Pro Lys Asp Thr Leu Met Ile
Ser Arg Thr Pro Glu Val Thr Cys Val Val Val Asp
Val Ser His Glu Asp Pro Glu Val Lys Phe Asn Trp
Tyr Val Asp Gly Val Glu Val His Asn Ala Lys Thr
Lys Pro Arg Glu Glu Gln Tyr Asn Ser Thr Tyr Arg
Val Val Ser Val Leu Thr Val Leu His Gln Asp Trp
Leu Asn Gly Lys Glu Tyr Lys Cys Lys Val Ser Asn
Lys Ala Leu Pro Ala Pro Ile Glu Lys Thr Ile Ser
Lys Ala Lys Gly Gln Pro Arg Glu Pro Gln Val Tyr
Thr Leu Pro Pro Ser Arg Glu Glu Met Thr Lys Asn
Gln Val Ser Leu Thr Cys Leu Val Lys Gly Phe Tyr
Pro Ser Asp Ile Ala Val Glu Trp Glu Ser Asn Gly
Gln Pro Glu Asn Asn Tyr Lys Thr Thr Pro Pro Val
Leu Asp Ser Asp Gly Ser Phe Phe Leu Tyr Ser Lys
Leu Thr Val Asp Lys Ser Arg Trp Gln Gln Gly Asn
Val Phe Ser Cys Ser Val Met His Glu Ala Leu His
Asn His Tyr Thr Gln Lys Ser Leu Ser Leu Ser Pro
Gly Lys

This protein of 422 amino acids, when expressed in a mammalian cell, is secreted from the cell. The mature secreted form of the protein contains amino acid residues 29-422. Of these residues, amino acid residues 29-192 represent the IL-18 binding protein portion of the molecule, and amino acid residues 193-422 represent the Fc portion of the molecule. The Fc region facilitates purification and dimerization of the fusion polypeptide. IL-18 antagonists may also comprise or be developed from IL-18 polynucleotide and/or polypeptide sequences.

Human IL-18 has been recombinantly produced from a cloned cDNA, as described in U.S. Pat. No. 5,891,663 and cloned genomic DNA, as disclosed in U.S. Pat. No. 6,060,283, each being incorporated by reference in their entirety. The full-length cDNA sequence is described herein as SEQ ID NO: 102 with the corresponding amino acid sequence described herein as SEQ ID NO: 103.

SEQ ID NO: 102
GTAAAGTAGA AATGAATTTA TTTTTCTTTG CAAACTAAGT ATCTGCTTGA
GACACATCTA TCTCACCATT GTCAGCTGAG GAAAAAAAAA AATGGTTCTC
ATGCTACCAA TCTGCCTTCA AAGAAATGTG GACTCAGTAG CACAGCTTTG
GAATGAAGAT GATCATAAGA GATACAAAGA AGAACCTCTA GCAAAAGATG
CTTCTCTATG CCTTAAAAAA TTCTCCAGCT CTTAGAATCT ACAAAATAGA
CTTTGCCTGT TTCATTGGTC CTAAGATTAG CATGAAGCCA TGGATTCTGT
TGTAGGGGGA GCGTTGCATA GGAAAAAGGG ATTGAAGCAT TAGAATTGTC
CAAAATCAGT AACACCTCCT CTCAGAAATG CTTTGGGAAG AAGCCTGGAA
GGTTCCGGGT TGGTGGTGGG GTGGGGCAGA AAATTCTGGA AGTAGAGGAG
ATAGGAATGG GTGGGGCAAG AAGACCACAT TCAGAGGCCA AAAGCTGAAA
GAAACCATGG CATTTATGAT GAATTCAGGG TAATTCAGAA TGGAAGTAGA
GTAGGAGTAG GAGACTGGTG AGAGGAGCTA GAGTGATAAA CAGGGTGTAG
AGCAAGACGT TCTCTCACCC CAAGATGTGA AATTTGGACT TTATCTTGGA
GATAATAGGG TTAATTAAGC ACAATATGTA TTAGCTAGGG TAAAGATTAG
TTTGTTGTAA CAAAGACATC CAAAGATACA GTAGCTGAAT AAGATAGAGA
ATTTTTCTCT CAAAGAAAGT CTAAGTAGGC AGCTCAGAAG TAGTATGGCT
GGAAGCAACC TGATGATATT GGGACCCCCA AC CTTCTTCA GTCTTGTACC
CATCATCCCC TAGTTGTTGA TCTCACTCAC ATAGTTGAAA ATCATCATAC
TTCCTGGGTT CATATCCCAG TTATCAAGAA AGGGTCAAGA GAAGTCAGGC
TCATTCCTTT CAAAGACTCT AATTGGAAGT TAAACACATC AATCCCCCTC
ATATTCCATT GACTAGAATT TAATCACATG GCCACACCAA GTGCAAGGAA
ATCTGGAAAA TATAATCTTT ATTCCAGGTA GCCATATGAC TCTTTAAAAT
TCAGAAATAA TATATTTTTA AAATATCATT CTGGCTTTGG TATAAAGAAT
TGATGGTGTG GGGTGAGGAG GCCAAAATTA AGGGTTGAGA GCCTATTATT
TTAGTTATTA CAAGAAATGA TGGTGTCATG AATTAAGGTA GACATAGGGG
AGTGCTGATG AGGAGCTGTG AATGGATTTT AGAAACACTT GAGAGAATCA
ATAGGACATG ATTTAGGGTT GGATTTGGAA AGGAGAAGAA AGTAGAAAAG
ATGATGCCTA CATTTTTCAC TTAGGCAATT TGTACCATTC AGTGAAATAG
GGAACACAGG AGGAAGAGCA GGTTTTGGTG TATACAAAGA GGAGGATGGA
TGACGCATTT CGTTTTGGAT CTGAGATGTC TGTGGAACGT CCTAGTGGAG
ATGTCCACAA ACTCTTCTAC ATGTGGTTCT GAGTTCAGGA CACAGATTTG
GGCTGGAGAT AGAGATATTG TAGGCTTATA CATAGAAATG GCATTTGAAT
CTATAGAGAT AAAAAGACAC ATCAGAGGAA ATGTGTAAAG TGAGAGAGGA
AAAGCCAAGT ACTGTGCTGG GGGGAATACC TACATTTAAA GGATGCAGTA
GAAAGAAGCT AATAAACAAC AGAGAGCAGA CTAACCAAAA GGGGAGAAGA
AAAACCAAGA GAATTCCACC GACTCCCAGG AGAGCATTTC AAGATTGAGG
GGATAGGTGT TGTGTTGAAT TTTGCAGCCT TGAGAATCAA GGGCCAGAAC
ACAGCTTTTA GATTTAGCAA CAAGGAGTTT GGTGATCTCA GTGAAAGCAG
CTTGATGGTG AAATGGAGGC AGAGGCAGAT TGCAATGAGT GAAACAGTGA
ATGGGAAGTG AAGAAATGAT ACAGATAATT CTTGCTAAAA GCTTGGCTGT
TAAAAGGAGG AGAGAAACAA GACTAGCTGC AAAGTGAGAT TGGGTTGATG
GAGCAGTTTT AAATCTCAAA ATAAAGAGCT TTGTGCTTTT TTGATTATGA
AAATAATGTG TTAATTGTAA CTAATTGAGG CAATGAAAAA AGATAATAAT
ATGAAAGATA AAAATATAAA AACCACCCAG AAATAATGAT AGCTACCATT
TTGATACAAT ATTTCTACAC TCCTTTCTAT GTATATATAC AGACACAGAA
ATGCTTATAT TTTTATTAAA AGGGATTGTA CTATACCTAA GCTGCTTTTT
CTAGTTAGTG ATATATATGG ACATCTCTCC ATGGCAACGA GTAATTGCAG
TTATATTAAG TTCATGATAT TTCACAATAA GGGCATATCT TTGCCCTTTT
TATTTAATCA ATTCTTAATT GGTGAATGTT TGTTTCCAGT TTGTTGTTGT
TATTAACAAT GTTCCCATAA GCATTCCTGT ACACCAATGT TCACACATTT
GTCTGATTTT TTCTTCAGGA TAAAACCCAG GAGGTAGAAT TGCTGGGTTG
ATAGAAGAGA AAGGATGATT GCCAAATTAA AGCTTCAGTA GAGGGTACAT
GCCGAGCACA AATGGGATCA GCCCTAGATA CCAGAAATGG CACTTTCTCA
TTTCCCCTTG GGACAAAAGG GAGAGAGGCA ATAACTGTGC TGCCAGAGTT
AAATTTGTAC GTGGAGTAGC AGGAAATCAT TTGCTGAAAA TGAAAACAGA
GATGATGTTG TAGAGGTCCT GAAGAGAGCA AAGAAAATTT GAAATTGCGG
CTATCAGCTA TGGAAGAGAG TGCTGAACTG GAAAACAAAA GAAGTATTGA
CAATTGGTAT GCTTGTAATG GCACCGATTT GAACGCTTGT GCCATTGTTC
ACCAGCAGCA CTCAGCAGCC AAGTTTGGAG TTTTGTAGCA GAAAGACAAA
TAAGTTAGGG ATTTAATATC CTGGCCAAAT GGTAGACAAA ATGAACTCTG
AGATCCAGCT GCACAGGGAA GGAAGGGAAG ACGGGAAGAG GTTAGATAGG
AAATACAAGA GTCAGGAGAC TGGAAGATGT TGTGATATTT AAGAACACAT
AGAGTTGGAG TAAAAGTGTA AGAAAACTAG AAGGGTAAGA GACCGGTCAG
AAAGTAGGCT ATTTGAAGTT AACACTTCAG AGGCAGAGTA GTTCTGAATG
GTAACAAGAA ATTGAGTGTG CCTTTGAGAG TAGGTTAAAA AACAATAGGC
AACTTTATTG TAGCTACTTC TGGAACAGAA GATTGTCATT AATAGTTTTA
GAAAACTAAA ATATATAGCA TACTTATTTG TCAATTAACA AAGAAACTAT
GTATTTTTAA ATGAGATTTA ATGTTTATTG TAG
SEQ ID NO: 103
Met Ala Ala Glu Pro Val Glu Asp Asn Cys Ile Asn Phe Val Ala Met Lys Phe Ile Asp Asn Thr
Leu Tyr Phe Ile Ala Ala Glu Asp Asp Glu Glu Asn Leu Glu Ser Asp Tyr Phe Gly Lys Leu Glu
Ser Lys Leu Ser Val Ile Arg Asn Leu Asn Asp Gln Val Leu Phe Ile Asp Gln Gly Asn Arg Pro
Leu Phe Glu Asp Met Thr Asp Ser Asp Cys Arg Asp Asp Asn Ala Pro Arg Thr Ile Phe Ile Ile
Ser Met Tyr Lys Asp Ser Gln Pro Arg Gly Met Ala Val Thr Ile Ser Val Lys Cys Glu Lys Ile Ser
Thr Leu Ser Cys Glu Asn Lys Ile Ile Ser Phe Lys Glu Met Asn Pro Pro Asp Asn Ile Lys Asp Thr
Lys Ser Asp Ile Ile Phe Phe Glu Arg Ser Val Pro Gly His Asp Asn Lys Met Gln Phe Glu Ser Ser
Ser Tyr Glu Gly Tyr Phe Leu Ala Cys Glu Lys Glu Arg Asp Leu Phe Lys Leu Ile Leu Lys Lys
Glu Asp Glu Leu Gly Asp Arg Ser Ile Met Phe Thr Val Gln Asn Glu Asp

The amino acid sequence for human IL-18 after proteolytic processing by caspase-1 (a.k.a. IL-1 converting enzyme, or ICE) is described herein as SEQ ID NO: 104. The amino acid sequence of human IL-18 after proteolytic processing by proteinase 3 (PR3) is described herein as SEQ ID NO:105. Commercially available recombinant human IL-18 is available, for example, from R&D Systems, Minneapolis, Minn.

SEQ ID NO: 104
Tyr Phe Gly Lys Leu Glu Ser Lys Leu Ser Val Ile
Arg Asn Leu Asn Asp Gln Val Leu Phe Ile Asp Gln
Gly Asn Arg Pro Leu Phe Glu Asp Met Thr Asp Ser
Asp Cys Arg Asp Asp Asn Ala Pro Arg Thr Ile Phe
Ile Ile Ser Met Tyr Lys Asp Ser Gln Pro Arg Gly
Met Ala Val Thr Ile Ser Val Lys Cys Glu Lys Ile
Ser Thr Leu Ser Cys Glu Asn Lys Ile Ile Ser Phe
Lys Glu Met Asn Pro Pro Asp Asn Ile Lys Asp Thr
Lys Ser Asp Ile Ile Phe Phe Gln Arg Ser Val Pro
Gly His Asp Asn Lys Met Gln Phe Glu Ser Ser Ser
Tyr Glu Gly Tyr Phe Leu Ala Cys Glu Lys Glu Arg
Asp Leu Phe Lys Leu Ile Leu Lys Lys Glu Asp Glu
Leu Gly Asp Arg Ser Ile Met Phe Thr Val Gln Asn
Glu Asp
SEQ ID NO: 105
Ile Arg Asn Leu Asn Asp Gln Val Leu Phe Ile Asp
Gln Gly Asn Arg Pro Leu Phe Glu Asp Met Thr Asp
Ser Asp Cys Arg Asp Asp Asn Ala Pro Arg Thr Ile
Phe Ile Ile Ser Met Tyr Lys Asp Ser Gln Pro Arg
Gly Met Ala Val Thr Ile Ser Val Lys Cys Glu Lys
Ile Ser Thr Leu Ser Cys Glu Asn Lys Ile Ile Ser
Phe Lys Glu Met Asn Pro Pro Asp Asn Ile Lys Asp
Thr Lys Ser Asp Ile Ile Phe Phe Gln Arg Ser Val
Pro Gly His Asp Asn Lys Met Gln Phe Glu Ser Ser
Ser Tyr Glu Gly Tyr Phe Leu Ala Cys Glu Lys Glu
Arg Asp Leu Phe Lys Leu Ile Leu Lys Lys Glu Asp
Glu Leu Gly Asp Arg Ser Ile Met Phe Thr Val Gln
Asn Glu Asp

In certain embodiments the target of the methods and compositions described herein is human IL-18, known as Interleukin IL-18. The amino acid sequence and nucleic acid sequence for human IL-18 are publicly available, see, e.g., GENBANK Accession No. NP_001230140.1 (SEQ ID NO: 107) and NM_001243211.2 (SEQ ID NO: 106) (both of which are incorporated by reference herein).

SEQ ID NO: 106
cctttgctcc cctggcgact gcctggacag tcagcaagga
attgtctccc agtgcatttt gccctcctgg ctgccaactc
tggctgctaa agcggctgcc acctgctgca gtctacacag
cttcgggaag aggaaaggaa cctcagacct tccagatcgc
ttcctctcgc aacaaactat ttgtcgcagg aataaagatg
gctgctgaac cagtagaaga caattgcatc aactttgtgg
caatgaaatt tattgacaat acgctttact ttatagaaaa
cctggaatca gattactttg gcaagcttga atctaaatta
tcagtcataa gaaatttgaa tgaccaagtt ctcttcattg
accaaggaaa tcggcctcta tttgaagata tgactgattc
tgactgtaga gataatgcac cccggaccat atttattata
agtatgtata aagatagcca gcctagaggt atggctgtaa
ctatctctgt gaagtgtgag aaaatttcaa ctctctcctg
tgagaacaaa attatttcct ttaaggaaat gaatcctcct
gataacatca aggatacaaa aagtgacatc atattctttc
agagaagtgt cccaggacat gataataaga tgcaatttga
atcttcatca tacgaaggat actttctagc ttgtgaaaaa
gagagagacc tttttaaact cattttgaaa aaagaggatg
aattggggga tagatctata atgttcactg ttcaaaacga
agactagcta ttaaaatttc atgccgggcg cagtggctca
cgcctgtaat cccagccctt tgggaggctg aggcgggcag
atcaccagag gtcaggtgtt caagaccagc ctgaccaaca
tggtgaaacc tcatctctac taaaaataca aaaaattagc
tgagtgtagt gacgcatgcc ctcaatccca gctactcaag
aggctgaggc aggagaatca cttgcactcc ggaggtagag
gttgtggtga gccgagattg caccattgcg ctctagcctg
ggcaacaaca gcaaaactcc atctcaaaaa ataaaataaa
taaataaaca aataaaaaat tca
SEQ ID NO: 107
MAAEPVEDNC INFVAMKFID NTLYFIENLE SDYFGKLESK
LSVIRNLNDQ VLFIDQGNRP LFEDMTDSDC RDNAPRTIFI
ISMYKDSQPR GMAVTISVKC EKISTLSCEN KIISFKEMNP
PDNIKDTKSD IIFFQRSVPG HDNKMQFESS SYEGYFLACE
KERDLFKLIL KKEDELGDRS IMFTVQNED

In certain embodiments the target of the methods and compositions described herein is human DR-18, also known as decoy resistant IL-18. The amino acid sequence and nucleic acid sequence for active (i.e., proteolytically processed) human DR-18 are publicly available, see, e.g. U.S. Patent Publication No. 2019/0070262 A1. This publication contains the below sequence for wild type hIL-18 (SEQ ID NO: 108)

SEQ ID NO: 108
Tyr Phe Gly Lys Leu Glu Ser Lys Leu Ser Val Ile
Arg Asn Leu Asn Asp Gln Val Leu Phe Ile Asp Gln
Gly Asn Arg Pro Leu Phe Glu Asp Met Thr Asp Ser
Asp Cys Arg Asp Asn Ala Pro Arg Thr Ile Phe Ile
Ile Ser Met Tyr Lys Asp Ser Gln Pro Arg Gly Met
Ala Val Thr Ile Ser Val Lys Cys Glu Lys Ile Ser
Thr Leu Ser Cys Glu Asn Lys Ile Ile Ser Phe Lys
Glu Met Asn Pro Pro Asp Asn Ile Lys Asp Thr Lys
Ser Asp Ile Ile Phe Phe Gln Arg Ser Val Pro Gly
His Asp Asn Lys Met Gln Phe Glu Ser Ser Ser Tyr
Glu Gly Tyr Phe Leu Ala Cys Glu Lys Glu Arg Asp
Leu Phe Lys Leu Ile Leu Lys Lys Glu Asp Glu Leu
Gly Asp Arg Ser Ile Met Phe Thr Val Gln Asn Glu
Asp

DR-18 polypeptides bind to, and signal through formation of, the IL-18Rα (IL-18 receptor α) and IL-18Rβ (IL-18 receptor β) receptor complex. DR-18 polypeptides display substantially reduced binding to IL-18BP, such as substantially reduced binding to IL-18BP relative to wild-type (WT) IL-18 (SEQ ID NO: 108), or do not bind to IL-18BP (i.e., as compared to the binding of IL-18BP to WT IL-18 (SEQ ID NO: 108)). In some embodiments, the DR-18 polypeptide employed binds to IL-18Rα and does not bind to IL-18BP. In some embodiments, the DR-18 polypeptide employed binds to IL-18Rα and has reduced binding to IL-18BP relative to WT IL-18.

Useful DR-18 polypeptides include, but are not limited to, e.g., those described in PCT Pub. No. WO2019/051015A1 and US Pat. Pub. No. US20210015891A1, the disclosures of which are incorporated herein by reference in their entirety.

In some embodiments, the DR-18 polypeptide comprises a modified IL-18 polypeptide comprising: (i) an amino acid sequence having 85% or more (e.g., at least 90%, 91%, 92%, 93%, 94%, 95%, 96%, 97%, 98%, 99%, or 100%) sequence identity with the IL-18 variant amino sequence set forth in any one of SEQ ID NO: 2-63; with or without (ii) mutations at amino acid positions Cysteine-38 and Cysteine-68 relative to WT IL-18 as set forth in SEQ ID NO: 108. DR-18 polypeptides, and compositions comprising DR-18 polypeptides, useful in the herein described methods are described in more detail below. Any of the herein described DR-18 polypeptides may find use in the methods of the present disclosure. In some instances, the methods will employ a DR-18 polypeptide (or a DR-18 composition comprising such a polypeptide) comprising an amino acid sequence having 85% or more (e.g., at least 90%, 91%, 92%, 93%, 94%, 95%, 96%, 97%, 98%, 99%, or 100%) sequence identity with the IL-18 variant amino sequence set forth in any one of SEQ ID NO: 2-63. In some instances, the methods will employ a DR-18 polypeptide (or a DR-18 composition comprising such a polypeptide) comprising an amino acid sequence having 85% or more (e.g., at least 90%, 91%, 92%, 93%, 94%, 95%, 96%, 97%, 98%, 99%, or 100%) sequence identity with the IL-18 variant amino sequence set forth in SEQ ID NO: 20, 63, 7, 17-19, 21, 22, 55-57, and 62.

In some instances, a useful DR-18 polypeptide comprises an amino acid sequence having at least 85%, 86%, 87%, 88%, 89%, 90%, 91%, 92%, 93%, 94%, 95%, 96%, 97%, 98%, or 99% sequence identity with SEQ ID NO:108 and/or at least one of SEQ ID NOs: 7, 17-22, 55-57, 62 and 63. In some instances, a useful DR-18 polypeptide comprises at least 1, 2, 3, 4, 5, 6, 7, 8, 9, 10, 12, 13, 14, or 15 mutated residues relative to SEQ ID NO:108. In some instances, a useful DR-18 polypeptide comprises up to 12, 11, or 10 mutated residues relative to SEQ ID NO:108.

In some instances, a useful DR-18 polypeptide comprises from 1 to 12, from 2 to 12, from 3 to 12, from 4 to 12, from 5 to 12, from 6 to 12, from 7 to 12, from 8 to 12, from 9 to 12, from 10 to 12, from 1 to 10, from 2 to 10, from 3 to 10, from 4 to 10, from 5 to 10, from 6 to 10, from 7 to 10, or from 8 to 10 mutated residues relative to SEQ ID NO:108.

In some instances, a useful DR-18 polypeptide comprises an amino acid sequence of any one of SEQ ID NOs: 7, 17-22, 55-57, 62 and 63 with 0, 1, 2, 3, 4, 5, or 6 additional mutated residues relative to SEQ ID NO:108. DR-18 polypeptides may or may not include (or be modified to include or exclude) one or more cysteine mutations relative to WT IL-18. For example, a stabilized IL-18 polypeptide can include mutations of two cysteine residues (e.g., C38 and C68) relative to human WT IL-18 (SEQ ID NO:108). In some cases, the mutation is a C to S substitution and as such the stabilized IL-18 polypeptide can in some cases comprise the mutations C38S and C68S. In some cases a stabilized IL-18 polypeptide comprises the mutation pair C38S/C68S, C38S/C68G, C38S/C68A, C38S/C68V, C38S/C68D, C38S/C68E, or C38S/C68N (e.g., in some cases C38S/C68G, C38S/C68A, C38S/C68V, C38S/C68D, C38S/C68E, or C38S/C68N; in some cases C38S/C68S, C38S/C68G, C38S/C68A, C38S/C68D, or C38S/C68N; and in some cases C38S/C68G, C38S/C68A, C38S/C68D, or C38S/C68N).

In some instances, a useful DR-18 polypeptide comprises one or more mutations at positions Y1, L5, K8, C38, M51, K53, S55, Q56, P57, G59, M60, C68, E77, Q103, S105, D110, N111, M113, V153, and N155 relative to the WT IL-18 amino acid sequence set forth in SEQ ID NO:108, including e.g., at least 1, at least 2, at least 3, at least 4, at least 5, at least 6, at least 7, at least, 8, at least 9, at least 10, at least 11, or at least 12 of the previously listed mutated positions. Accordingly, useful DR-18 polypeptides can have one or any combination of mutations at the listed positions, with or without additional mutated residues at other positions. For example, useful polypeptides can have a mutation at position Y1, L5, K8, C38, M51, K53, S55, Q56, P57, G59, M60, C68, E77, Q103, S105, D110, N111, M113, V153, or N155 only; mutations at all positions Y1, L5, K8, C38, M51, K53, S55, Q56, P57, G59, M60, C68, E77, Q103, S105, D110, N111, M113, V153, and N155; or any combinations having more than one but less than all (e.g., 2, 3, 4, 5, 6, 7, 8, 9, 10, 11, 12, 13, 14, 15, 16, 17, 18 or 19) mutations at these positions.

A DR-18 polypeptide may comprise at least 12 mutations, at least 11 mutations, at least 10 mutations, at least 9 mutations, at least 8 mutations, at least 7 mutations, at least 6 mutations, at least 5 mutations, at least 4 mutations, at least 3 mutations, at least 2 mutations, or at least 1 mutation relative to the wild-type IL-18 amino acid sequence set forth in SEQ ID NO:108, optionally wherein the at least 10, 9, 8, 7, 6, 5, 4, 3, 2, or 1 mutations are at positions selected from Y1, L5, K8, C38, M51, K53, S55, Q56, P57, G59, M60, C68, E77, Q103, S105, D110, N111, M113, V153, and N155. In some instances, the residue at position (pos.) 1 is Y, R or H; the residue at pos. 5 is L, H, I or Y; the residue at pos. 8 is K, Q or R; the residue at pos. 38 is C or S; the residue at pos. 51 is M, T, K, D, N, E or R; the residue at pos. 53 is K, R, G, S or T; the residue at pos. 55 is S, K or R; the residue at pos. 56 is Q, E, A, R, V, G, K, L or R; the residue at pos. 57 is P, L, G, A or K; the residue at pos. 59 is G, A or T; the residue at pos. 60 is M, K, Q, R or L; the residue at pos. 68 is C, S, G, A, V, D, E or N; the residue at pos. 76 is C or S; the residue at pos. 77 is E or D; the residue at pos. 103 is Q, E, K, P, A or R; the residue at pos. 105 is S, D, N, R, K or A; the residue at pos. 110 is D, K, H, N, Q, E, S or G; the residue at pos. 111 is N, H, Y, D, R, S or G; the residue at pos. 113 is M, V, R, T or K; the residue at pos. 127 is C or S; the residue at pos. 153 is V, I, T or A; and the residue at pos. 155 is N, K or H.

In some instances, the DR-18 polypeptide comprises one or more substitutions relative to WT human IL-18 (SEQ ID NO: 108), including 1, 2, 3, 4, 5, 6, 7, 8, 9, 10 or more substitutions selected from: Y1H, Y1R, L5H, L5I, L5Y, K8Q, K8R, M51T, M51K, M51D, M51N, M51E, M51R, K53R, K53G, K53S, K53T, S55K, S55R, Q56E, Q56A, Q56R, Q56V, Q56G, Q56K, Q56L, P57L, P57G, P57A, P57K, G59T, G59A, M60K, M60Q, M60R, M60L, E77D, Q103E, Q103K, Q103P, Q103A, Q103R, S105R, S105D, S105K, S105N, S105A, D110H, D110K, D110N, D110Q, D110E, D110S, D110G, N111H, N111Y, N111D, N111R, N111S, N111G, M113V, M113R, M113T, M113K, V153I, V153T, V153A, N155K, and N155H.

In some instances, the DR-18 polypeptide comprises one or more substitutions relative to WT human IL-18 (SEQ ID NO: 108) such that, e.g., the amino acid at pos. 1 is not Y and, e.g., may be R or H; the amino acid at pos. 5 is not L and, e.g., may be H, I or Y; the amino acid at pos. 8 is not K and, e.g., may be Q or R; the amino acid at pos. 38 is not C and, e.g., may be S; the amino acid at pos. 51 is not M and, e.g., may be T, K, D, N, E or R; the amino acid at pos. 53 is not K and, e.g., may be R, G, S or T; the amino acid at pos. 55 is not S and, e.g., may be K or R; the amino acid at pos. 56 is not Q and, e.g., may be E, A, R, V, G, K, L or R; the amino acid at pos. 57 is not P and, e.g., may be L, G, A or K; the amino acid at pos. 59 is not G and, e.g., may be A or T; the amino acid at pos. 60 is not M and, e.g., may be K, Q, R or L; the amino acid at pos. 68 is not C and, e.g., may be S, G, A, V, D, E or N; the amino acid at pos. 76 is not C and, e.g., may be S; the amino acid at pos. 77 is not E and, e.g., may be D; the amino acid at pos. 103 is not Q and, e.g., may be E, K, P, A or R; the amino acid at pos. 105 is not S and, e.g., may be D, N, R, K or A; the amino acid at pos. 110 is not D and, e.g., may be K, H, N, Q, E, S or G; the amino acid at pos. 111 is not N and, e.g., may be H, Y, D, R, S or G; the amino acid at pos. 113 is not M and, e.g., may be V, R, T or K; the amino acid at pos. 127 is not C and, e.g., may be S; the amino acid at pos. 153 is not V and, e.g., may be an I, T or A; and/or the amino acid at pos. 155 is not N and, e.g., may be K or H.

In some instances, the DR-18 polypeptide comprises substitution mutations, relative to WT human IL-18 as set forth in SEQ ID NO:108, at positions: (i) M51, M60, S105, D110, and N111; (ii) M51, S55, G59, M60, S105, D110, N111, and V153; (iii) Y1, M51, M60, S105, D110, and N111; (iv) Y1, M51, K53, M60, S105, D1110, and N111; (v) K8, M51, S55, G59, M60, S105,1D110, and N155; (vi) K8, M51, S55, G59, M60, S105, D110, N111, and V153; (vii) L5, M51, K53, M60, S105, D110, and V153; (viii) L5, M51, S55, G59, M60, S105, D110, N111, and N155; (ix) L5, M51, S55, M60, Q103, S105, D110, N111, and V153; (x) L5, M51, S55, M60, S105, D110, N111, V153, and N155; (xi) L5, M51, S55, G59, M60, S105, D110, N111, V153, and N155; (xii) L5, K8, M51, S55, M60, S105, N111, V153, and N155; (xiii) L5, K8, M51, K53, M60, S105, D110, N111, and N155; (xiv) Y1, L5, M51, K53, M60, S105, D110, and N155; (xv) Y1, M51, K53, G59, M60, S105, D110, N111, V153, and N155; (xvi) Y1, K8, M51, K53, M60, Q103, S105, D110, N111, and N155; (xvii) Y1, K8, M51, M60, S105, D110, and N111; (xviii) Y1, L5, M51, K53, M60, Q103, S105, D110, and N111; (xix) Y1, K8, M51, K53, G59, M60, Q103, S105, D110, N111, V153, and N155; (xx) Y1, K8, M51, K53, G59, M60, S105, D110, N111, and N155; (xxi) Y1, K8, M51, G59, M60, Q103, S105, D110, N111, V153, and N155; (xxii) Y1, L5, M51, G59, M60, E77, S105, D110, and N111; (xxiii) M51, Q56, P57, M60, Q103, S105, D110, N111, and M113; (xxiv) M51, Q56, P57, M60, Q103, S105, D110, and M113; (xxv) M51, K53, Q56, P57, M60, D110, and N111; (xxvi) M51, K53, Q56, P57, M60, Q103, S105, D110, N111, and M113; (xxvii) M51, K53, Q56, M60, Q103, S105, D110, N111, and M113; (xxviii) M51, K53, Q56, P57, Q103, S105, D110, N111, and M113; (xxix) M51, K53, Q56, P57, M60, S105, D110, and N111; (xxx) M51, K53, Q56, P57, M60, Q103, D110, N111, and M113; (xxxi) M51, Q56, P57, M60, Q103, D110, N111, and M113; or (xxxii) M51, K53, Q56, S105, D110, and N111.

In some instances, the DR-18 polypeptide comprises substitution, relative to WT human IL-18 as set forth in SEQ ID NO:108, of: (i) M51T, M60K, S105D, D110K, and N111H; (ii) M51T, S55K, G59A, M60K, S105D, D110K, N111H, and V153I; (iii) Y1R, M51T, M60K, S105D, D110K, and N111H; (iv) YR, M51T, K53R, M60K, S105N, D110K, and N111Y; (v) K8Q, M51T, S55K, G59T, M60K, S105R, D110H, and N155K; (vi) K8R, M51K, S55K, G59A, M60Q, S105D, D110K, N111H, and V153I; (vii) K8R, M51D, S55K, G59A, M60X, S105D, D110K, N111H, and V153I; (viii) L5H, M51T, K53R, M60K, S105D, D110N, and V153T; (ix) L5I, M51K, S55K, G59A, M60Q, S105K, D110Q, N111H, and N155K; (x) L5I, M51T, S55R, M60K, Q103E, S105D, D110H, N111H, and V153I; (xi) L5I, M51T, S55K, M60K, S105D, D110K, N111H, V153T, and N155H; (xii) L5I, M51T, S55K, G59A, M60K, S105R, D110H, N111H, V153I, and N155K; (xiii) L5I, K8R, M51T, S55K, M60K, S105D, N111Y, V153I, and N155K; (xiv) L5Y, K8R, M51T, K53R, M60K, S105D, D110E, N111H, and N155K; (xv) Y1H, L5Y, M51T, K53R, M60K, S105D, D110H, and N155K; (xvi) Y1R, M51T, K53R, G59A, M60K, S105D, D110Q, N111H, V153A, and N155K; (xvii) Y1R, K8R, M51D, K53R, M60R, Q103K, S105N, D110K, N111Y, and N155H; (xviii) Y1R, K8R, M51N, K53R, M60Q, Q103K, S105R, D110N, N111H, and N155K; (xix) Y1R, K8R, M51T, M60K, S105D, D110K, and N111H; (xx) Y1R, L5H, M51T, K53R, M60K, Q103E, S105N, D110K, and N111Y; (xxi) Y1R, K8R, M51T, K53R, G59A, M60K, Q103E, S105D, D110Q, N111H, V153I, and N155X; (xxii) Y1R, K8R, M51T, K53R, G59T, M60K, S105N, D110H, N111D, and N155H; (xxiii) Y1R, K8R, M51T, G59A, M60K, Q103E, S105D, D110Q, N111H, V153I, and N155K; (xxiv) Y1R, L5Y, M51T, G59T, M60K, E77D, S105D, D110K, and N111H; (xxv) Y1R, K8R, M51T, K53R, G59T, M60K, S105K, D110N, N111H, and N155K; (xxvi) M51E, Q56E, P57L, M60R, Q103P, S105A, D110N, N111R, and M113V; (xxvii) M51K, Q56A, P57G, M60L, Q103E, S105D, D110S, and M113V; (xxviii) M51K, K53G, Q56A, P57A, M60L, D110K, and N111R; (xxix) M51K, K53G, Q56R, P57G, M60L, Q103E, S105D, D110N, N111S, and M113R; (xxx) M51K, K53G, Q56V, M60L, Q103A, S105A, D110S, N111R, and M113T; (xxxi) M51K, K53S, Q56G, P57A, M60L, Q103A, S105A, D110G, N111R, and M113T; (xxxii) M51K, K53S, Q56K, P57A, Q103A, S105D, D110S, N111S, and M113R; (xxxiii) M51K, K53S, Q56L, P57A, M60L, S105D, D110S, and N111R; (xxxiv) M51K, K53S, Q56R, P57A, M60L, S105N, D110G, and N111R; (xxxv) M51K, K53S, Q56R, P57A, M60L, Q103A, D110G, N111R, and M113T; (xxxvi) M51K, K53S, Q56R, P57A, M60L, Q103A, S105D, D110S, N111G, and M113R; (xxxvii) M51K, K53T, Q56R, M60L, Q103E, S105D, D110S, N111S, and M113K; (xxxviii) M51K, K53T, Q56R, P57A, Q103E, S105D, D110N, N111D, and M113R; (xxxix) M51R, Q56G, P57K, M60L, Q103R, D110S, N111R, and M113V; (xl) M51K, K53G, Q56G, P57A, M60L, Q103E, S105D, D110S, N111G, and M113V; or (xli) M51K, K53G, Q56R, S105A, D110N, and N111R.

In some instances, the engineered IL-18 polypeptide is a DR IL-18 polypeptide that comprises 100% sequence identity to an amino acid sequence selected from SEQ ID NOs: 7, 17-22, 55-57, 62 and 63. In some instances, the engineered IL-18 polypeptide is a DR IL-18 polypeptide that comprises 100% sequence identity to SEQ ID NO: 20.

Useful DR-18 polypeptides include those listed by sequence in the following table:

| | SEQ ID |
|---|---|
| DR-18 Amino Acid Sequence | NO.: |
| YFGKLESKLSVIRNLNDQVLFIDQGNRPLFEDMTDSDSRDNAPRTIFIISKYSDSLARGL | 2 |
| AVTISVKSEKISTLSSENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDCRDNAPRTIFIISKYSDSLARGL | 3 |
| AVTISVKSEKISTLSSENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDSRDNAPRTIFIISKYSDSLARGL | 4 |
| AVTISVKCEKISTLSSENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDSRDNAPRTIFIISKYSDSLARGL | 5 |
| AVTISVKSEKISTLSCENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDSRDNAPRTIFIISKYSDSLARGL | 6 |
| AVTISVKSEKISTLSSENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 7 |
| AVTISVKSEKISTLSCENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 8 |
| AVTISVKCEKISTLSSENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 9 |
| AVTISVKCEKISTLSCENKIISFKEMNPPDNIKDTKSDIIFFQRDVPGHSRKMQFESSSYE | |
| GYFLASEKERDLFKLILKKEDELGDRSIMFTVQNED | |
| YFGKLESKLSVIRNLNDQVLFIDQGNRPLFEDMTDSDCRDNAPRTIFIISKYSDSLARGL | 10 |
| AVTISVKSEKISTLSSENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSLARGL | 11 |
| AVTISVKSEKISTLSCENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDCRDNAPRTIFIISKYSDSLARGL | 12 |
| AVTISVKCEKISTLSSENKIISFKEMNPPDNIKDTKSDIIFFQRDVPGHSRKMQFESSSYEG | |
| YFLASEKERDLFKLILKKEDELGDRSIMFTVQNED | |
| YFGKLESKLSVIRNLNDQVLFIDQGNRPLFEDMTDSDSRDNAPRTIFIISKYSDSLARGL | 13 |
| AVTISVKCEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISKYSDSLARGL | 14 |
| AVTISVKSEKISTLSCENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSLARGL | 15 |
| AVTISVKCEKISTLSSENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSLARGL | 16 |
| AVTISVKCEKISTLSCENKIISFKEMNPPDNIKDTKSDIIFFQRDVPGHSRKMQFESSSYE | |
| GYFLASEKERDLFKLILKKEDELGDRSIMFTVQNED | |
| YFGKLESKLSVIRNLNDQVLFIDQGNRPLFEDMTDSDSRDNAPRTIFIISKYSDSLARGL | 17 |
| AVTISVKGEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 18 |
| AVTISVKAEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 19 |
| AVTISVKVEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 20 |
| AVTISVKDEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 21 |
| AVTISVKEEKISTLSCENKIISFKEMNPPDNIKDTKSDIIFFQRDVPGHSRKMQFESSSYEG | |
| YFLACEKERDLFKLILKKEDELGDRSIMFTVQNED | |
| YFGKLESKLSVIRNLNDQVLFIDQGNRPLFEDMTDSDSRDNAPRTIFIISKYSDSLARGL | 22 |
| AVTISVKNEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISTYKDSQPRGK | 23 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISTYKDKQPRAK | 24 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTIQNED | |
| RFGKLESKLSVIRNLNDQVLFIDQGNRPLFEDMTDSDCRDNAPRTIFIISTYKDSQPRGK | 25 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTVQNED | |
| RFGKLESKLSVIRNLNDQVLFIDQGNRPLFEDMTDSDCRDNAPRTIFIISTYRDSQPRGK | 26 |
| AVTISVKCEKISTLSCENKIISFKEMNPPDNIKDTKSDIIFFQRNVPGHKYKMQFESSSYE | |
| GYFLACEKERDLFKLILKKEDELGDRSIMFTVQNED | |
| YFGKLESQLSVIRNLNDQVLFIDQGNRPLFEDMTDSDCRDNAPRTIFIISTYKDKQPRTK | 27 |
| AVTISVKCEKISTLSCENKIISFKEMNPPDNIKDTKSDIIFFQRRVPGHHNKMQFESSSYE | |
| GYFLACEKERDLFKLILKKEDELGDRSIMFTVQKED | |
| YFGKLESRLSVIRNLNDQVLFIDQGNRPLFEDMTDSDCRDNAPRTIFIISKYKDKQPRAQ | 28 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTIQNED | |
| YFGKLESRLSVIRNLNDQVLFIDQGNRPLFEDMTDSDCRDNAPRTIFIISDYKDKQPRAX | 29 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTIQNED | |
| YFGKHESKLSVIRNLNDQVLFIDQGNRPLFEDMTDSDCRDNAPRTIFIISTYRDSQPRGK | 30 |
| AVTISVKCEKISTLSCENKIISFKEMNPPDNIKDTKSDIIFFQRDVPGHNNKMQFESSSYE | |
| GYFLACEKERDLFKLILKKEDELGDRSIMFTTQNED | |
| YFGKIESKLSVIRNLNDQVLFIDQGNRPLFEDMTDSDCRDNAPRTIFIISKYKDKQPRAQ | 31 |
| AVTISVKCEKISTLSCENKIISFKEMNPPDNIKDTKSDIIFFQRKVPGHQHKMQFESSSYE | |
| GYFLACEKERDLFKLILKKEDELGDRSIMFTVQKED | |
| YFGKIESKLSVIRNLNDQVLFIDQGNRPLFEDMTDSDCRDNAPRTIFIISTYKDRQPRGK | 32 |
| AVTISVKCEKISTLSCENKIISFKEMNPPDNIKDTKSDIIFFERDVPGHHHKMQFESSSYE | |
| GYFLACEKERDLFKLILKKEDELGDRSIMFTIQNED | |
| YFGKIESKLSVIRNLNDQVLFIDQGNRPLFEDMTDSDCRDNAPRTIFIISTYKDKQPRGK | 33 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTTQHED | |
| YFGKIESKLSVIRNLNDQVLFIDQGNRPLFEDMTDSDCRDNAPRTIFIISTYKDKQPRAK | 34 |
| AVTISVKCEKISTLSCENKIISFKEMNPPDNIKDTKSDIIFFQRRVPGHHHKMQFESSSYE | |
| GYFLACEKERDLFKLILKKEDELGDRSIMFTIQKED | |
| YFGKIESRLSVIRNLNDQVLFIDQGNRPLFEDMTDSDCRDNAPRTIFIISTYKDKQPRGK | 35 |
| AVTISVKCEKISTLSCENKIISFKEMNPPDNIKDTKSDIIFFQRDVPGHDYKMQFESSSYE | |
| GYFLACEKERDLFKLILKKEDELGDRSIMFTIQKED | |
| YFGKYESRLSVIRNLNDQVLFIDQGNRPLFEDMTDSDCRDNAPRTIFIISTYRDSQPRGK | 36 |
| AVTISVKCEKISTLSCENKIISFKEMNPPDNIKDTKSDIIFFQRDVPGHEHKMQFESSSYE | |
| GYFLACEKERDLFKLILKKEDELGDRSIMFTVQKED | |
| HFGKYESKLSVIRNLNDQVLFIDQGNRPLFEDMTDSDCRDNAPRTIFIISTYRDSQPRGK | 37 |
| AVTISVKCEKISTLSCENKIISFKEMNPPDNIKDTKSDIIFFQRDVPGHHNKMQFESSSYE | |
| GYFLACEKERDLFKLILKKEDELGDRSIMFTVQKED | |
| RFGKLESKLSVIRNLNDQVLFIDQGNRPLFEDMTDSDCRDNAPRTIFIISTYRDSQPRAK | 38 |
| AVTISVKCEKISTLSCENKIISFKEMNPPDNIKDTKSDIIFFQRDVPGHQHKMQFESSSYE | |
| GYFLACEKERDLFKLILKKEDELGDRSIMFTAQKED | |
| RFGKLESRLSVIRNLNDQVLFIDQGNRPLFEDMTDSDCRDNAPRTIFIISDYRDSQPRGR | 39 |
| AVTISVKCEKISTLSCENKIISFKEMNPPDNIKDTKSDIIFFKRNVPGHKYKMQFESSSYE | |
| GYFLACEKERDLFKLILKKEDELGDRSIMFTVQHED | |
| RFGKLESRLSVIRNLNDQVLFIDQGNRPLFEDMTDSDCRDNAPRTIFIISNYRDSQPRGQ | 40 |
| AVTISVKCEKISTLSCENKIISFKEMNPPDNIKDTKSDIIFFKRRVPGHNHKMQFESSSYE | |
| GYFLACEKERDLFKLILKKEDELGDRSIMFTVQKED | |
| RFGKLESRLSVIRNLNDQVLFIDQGNRPLFEDMTDSDCRDNAPRTIFIISTYKDSQPRGK | 41 |
| AVTISVKCEKISTLSCENKIISFKEMNPPDNIKDTKSDIIFFQRDVPGHKHKMQFESSSYE | |
| GYFLACEKERDLFKLILKKEDELGDRSIMFTVQNED | |
| RFGKHESKLSVIRNLNDQVLFIDQGNRPLFEDMTDSDCRDNAPRTIFIISTYRDSQPRGK | 42 |
| AVTISVKCEKISTLSCENKIISFKEMNPPDNIKDTKSDIIFFERNVPGHKYKMQFESSSYE | |
| GYFLACEKERDLFKLILKKEDELGDRSIMFTVQNED | |
| RFGKLESRLSVIRNLNDQVLFIDQGNRPLFEDMTDSDCRDNAPRTIFIISTYRDSQPRAK | 43 |
| AVTISVKCEKISTLSCENKIISFKEMNPPDNIKDTKSDIIFFERDVPGHQHKMQFESSSYE | |
| GYFLACEKERDLFKLILKKEDELGDRSIMFTIQXED | |
| RFGKLESRLSVIRNLNDQVLFIDQGNRPLFEDMTDSDCRDNAPRTIFIISTYRDSQPRTK | 44 |
| AVTISVKCEKISTLSCENKIISFKEMNPPDNIKDTKSDIIFFQRNVPGHHDKMQFESSSYE | |
| GYFLACEKERDLFKLILKKEDELGDRSIMFTVQHED | |
| RFGKLESRLSVIRNLNDQVLFIDQGNRPLFEDMTDSDCRDNAPRTIFIISTYKDSQPRAK | 45 |
| AVTISVKCEKISTLSCENKIISFKEMNPPDNIKDTKSDIIFFERDVPGHQHKMQFESSSYE | |
| GYFLACEKERDLFKLILKKEDELGDRSIMFTIQKED | |
| RFGKYESKLSVIRNLNDQVLFIDQGNRPLFEDMTDSDCRDNAPRTIFIISTYKDSQPRTK | 46 |
| AVTISVKCEKISTLSCDNKIISFKEMNPPDNIKDTKSDIIFFQRDVPGHKHKMQFESSSYE | |
| GYFLACEKERDLFKLILKKEDELGDRSIMFTVQNED | |
| RFGKLESRLSVIRNLNDQVLFIDQGNRPLFEDMTDSDCRDNAPRTIFIISTYRDSQPRTK | 47 |
| AVTISVKCEKISTLSCENKIISFKEMNPPDNIKDTKSDIIFFQRKVPGHNHKMQFESSSYE | |
| GYFLACEKERDLFKLILKKEDELGDRSIMFTVQKED | |
| YFGKLESKLSVIRNLNDQVLFIDQGNRPLFEDMTDSDCRDNAPRTIFIISEYKDSELRGR | 48 |
| AVTISVKCEKISTLSCENKIISFKEMNPPDNIKDTKSDIIFFPRAVPGHNRKVQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYKDSAGRGL | 49 |
| AVTISVKCEKISTLSCENKIISFKEMNPPDNIKDTKSDIIFFERDVPGHSNKVQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYGDSAARGL | 50 |
| AVTISVKCEKISTLSCENKIISFKEMNPPDNIKDTKSDIIFFQRSVPGHKRKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISKYGDSRGRGL | 51 |
| AVTISVKCEKISTLSCENKIISFKEMNPPDNIKDTKSDIIFFERDVPGHNSKRQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYGDSVPRGL | 52 |
| AVTISVKCEKISTLSCENKIISFKEMNPPDNIKDTKSDIIFFARAVPGHSRKTQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSGARGL | 53 |
| AVTISVKCEKISTLSCENKIISFKEMNPPDNIKDTKSDIIFFARAVPGHGRKTQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSKARGM | 54 |
| AVTISVKCEKISTLSCENKIISFKEMNPPDNIKDTKSDIIFFARDVPGHSSKRQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSLARGL | 55 |
| AVTISVKCEKISTLSCENKIISFKEMNPPDNIKDTKSDIIFFQRDVPGHSRKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISKYSDSRARGL | 56 |
| AVTISVKCEKISTLSCENKIISFKEMNPPDNIKDTKSDIIFFQRNVPGHGRKMQFESSSYE | |
| GYFLACEKERDLFKLILKKEDELGDRSIMFTVQNED | |
| YFGKLESKLSVIRNLNDQVLFIDQGNRPLFEDMTDSDCRDNAPRTIFIISKYSDSRARGL | 57 |
| AVTISVKCEKISTLSCENKIISFKEMNPPDNIKDTKSDIIFFARSVPGHGRKTQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYSDSRARGL | 58 |
| AVTISVKCEKISTLSCENKIISFKEMNPPDNIKDTKSDIIFFARDVPGHSGKRQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYTDSRPRGL | 59 |
| AVTISVKCEKISTLSCENKIISFKEMNPPDNIKDTKSDIIFFERDVPGHSSKKQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYTDSRARGM | 60 |
| AVTISVKCEKISTLSCENKIISFKEMNPPDNIKDTKSDIIFFERDVPGHNDKRQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISRYKDSGKRGL | 61 |
| AVTISVKCEKISTLSCENKIISFKEMNPPDNIKDTKSDIIFFRRSVPGHSRKVQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYGDSGARGL | 62 |
| AVTISVKCEKISTLSCENKIISFKEMNPPDNIKDTKSDIIFFERDVPGHSGKVQFESSSYEG | |
| YFLACEKERDLFKLILKKEDELGDRSIMFTVQNED | |
| YFGKLESKLSVIRNLNDQVLFIDQGNRPLFEDMTDSDCRDNAPRTIFIISKYGDSRPRGM | 63 |
| AVTISVKCEKISTLSCENKIISFKEMNPPDNIKDTKSDIIFFQRAVPGHNRKMQFESSSYE | |
| GYFLACEKERDLFKLILKKEDELGDRSIMFTVQNED | |

Also useful, in some embodiments, are nucleic acids that encode a DR-18 polypeptide, including e.g., one of the DR-18 polypeptides included above. For example, in some instances, the methods of the present disclosure may include administering to a subject in need thereof, a nucleic acid (e.g., a mRNA or a DNA) that encodes the DR-18 polypeptide. Such nucleic acids may be administered “naked” (e.g., in the case of naked linear or circular DNA or RNA) or encased or encapsulated, e.g., in a viral particle or non-viral vector. Useful viral and non-viral vectors, and useful components thereof, include e.g., adenoviral vectors, AAV vectors, lentiviral vectors, lipid nanoparticle vectors, and the like. In some instances, the methods of the present disclosure may include administering to a subject in need thereof, a cell (such as e.g., a CD8+ T cell) that comprises an introduced nucleic acid (e.g., a mRNA or a DNA) that encodes the DR-18 polypeptide. Useful cells may be autologous or allogeneic with respect to the subject being treated. In such cells, such introduced nucleic acids may be integrated into the genome of the cell (e.g., through the use of an integrating vector, site directed nucleases, homologous recombination, or any combination thereof or the like) or may be extrachromosomal. Cells engineered to contain a sequence encoding a DR-18 polypeptide and express the DR-18, (constitutively, conditionally or inducibly) may be referred to as “armored with DR-18” or a “DR-18 armored cell”.

In one embodiment, IL-18 agonists are polypeptide mimetics, such as, but not limited to peptidomimetics, peptibodies and/or mimotopes developed from the polypeptide sequence of IL-18 (listed above). Polypeptide mimetics may be developed through techniques known in the art, such as combinatorial peptide libraries. Polypeptide mimetics are peptide-containing molecules which mimic elements of protein secondary structure. In one aspect, an IL-18 polypeptide mimetic based on the amino acid sequence of IL-18 will bind to IL-18 receptor without activating the pro-inflammatory signaling pattern of the IL-18 receptor and promote its immune protect function. As such, IL-18 polypeptide mimetics may be used to treat inflammatory and/or immunoregulatory processes associated with inhibiting disease associated immunopathology resulting from pathogenic, autoreactive CD4+ T cell signaling.

Biological activity, in this instance, is the capacity to bind its cognate partner. The present disclosure additionally provides for the use of IL-18 agonists in the manufacture of a medicament for the treatment of immunopathology resulting from CD4+ T cell disease symptoms resulting from aberrant immune cell signaling. Further provided is the use of polynucleotides encoding IL-18 or variants thereof for increasing unopposed IL-18, in the manufacture of a medicament for the treatment of inhibiting immunopathology resulting from pathogenic or autoreactive CD4+ T cell activity.

Various means for attaching chemical moieties useful for increasing bio-availability and/or pharmacokinetic half-life are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

A preferred polymer vehicle is polyethylene glycol (PEG). The PEG group may be of any convenient molecular weight and may be linear or branched. The average molecular weight of the PEG will preferably range from about 2 kiloDalton (“kD”) to about 100 kD, more preferably from about 5 kD to about 50 kD, most preferably from about 5 kD to about 10 kD. The PEG groups will generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group).

A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water-soluble polymer which may be used for protein modification. Dextrans are polysaccharide polymers comprised of individual subunits of glucose predominantly linked by al-6 linkages. The dextran itself is available in many molecular weight ranges and is readily available in molecular weights from about 1 kD to about 70 kD. Dextran is a suitable water-soluble polymer for use in the present disclosure as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference in its entirety. Dextran of about 1 kD to about 20 kD is preferred when dextran is used as a vehicle in accordance with the present disclosure.

### “Decoy-to-the-Decoy” (D2D) Engineered IL-18 Polypeptides as IL-18BP Antagonists

As described above, wild type IL-18 binds to both IL-18R (to signal through the receptor) and IL-18BP (which inhibits IL-18 by preventing it from binding to IL-18R). DR-18 variants bind to IL-18R but have reduced binding to (and in some cases do not bind to) IL-18BP. Also useful are so called “decoy-to-the-decoy IL-18” (“D2D IL-18”) variants. Such D2D variants bind to IL-18BP but have reduced binding to (and in some cases do not bind to) IL-18R. Binding affinity may be comparable binding affinity, such as comparable binding affinity to IL-18R as wild type IL-18, or may be compared to wild type IL-18, such as exhibiting decreased binding affinity as compared to wild type IL-18.

Useful D2D polypeptides include, but are not limited to, e.g., those described in Clark et al.²⁹, PCT Pub. No. WO2019/051015A1 and US Pat. Pub. No. US20210015891A1, the disclosures of which are incorporated herein by reference in their entirety. D2D polypeptides may or may not include (or be modified to include or exclude) one or more cysteine mutations relative to WT IL-18. For example, a stabilized IL-18 polypeptide can include mutations of two cysteine residues (e.g., C38 and C68) relative to human WT IL-18 (SEQ ID NO:108). In some cases, the mutation is a C to S substitution and as such the stabilized IL-18 polypeptide can in some cases comprise the mutations C38S and C68S. In some cases a stabilized IL-18 polypeptide comprises the mutation pair C38S/C68S, C38S/C68G, C38S/C68A, C38S/C68V, C38S/C68D, C38S/C68E, or C38S/C68N (e.g., in some cases C38S/C68G, C38S/C68A, C38S/C68V, C38S/C68D, C38S/C68E, or C38S/C68N; in some cases C38S/C68S, C38S/C68G, C38S/C68A, C38S/C68D, or C38S/C68N; and in some cases C38S/C68G, C38S/C68A, C38S/C68D, or C38S/C68N).

In some instances, useful D2D's include a D2D IL-18 polypeptide that comprises 10000 sequence identity to an amino acid sequence selected from SEQ ID NOs: 64-97. In some instances, useful D2D's include a D2D IL-18 polypeptide that comprises at least 85%, at least 86%, at least 87%, at least 88%, at least 89%, at least 90%, at least 91%, at least 92%, at least 93% at least 94% at least 95% at least 96%, at least 97% at least 98%, or at least 99% sequence identity to an amino acid sequence selected from SEQ ID NOs: 64-97.

Useful D2D polypeptides include those listed by sequence in the following table:

| | SEQ ID |
|---|---|
| D2D Amino Acid Sequence | NO.: |
| HFGKLESKLSVIRNLNGQVLFIDQGNRPLFKDMTASDCRANAPRTIFIISFYKDSQPRGM | 64 |
| AVTISVKCEKISTLSCENKIISFKEMNPPDNIKDTKSDIIFFIRSVPGADNKFQFESSSYEG | |
| YFLACEKERDLFKLILKKEDELGDRSIMFTVQNED | |
| DFGKLESKLSVIRNLNDQVLFIDQGNRPLFADMTDNPCRSNAPRTIFIISFYKDSQPRGIA | 65 |
| VTISVKCEKISTLSCENKIISFKEMNPPDNIKDTKSDIIFFLRSVPGPDNKMQFESSSYEGY | |
| FLACEKERDLFKLILKKEDELGDRSIMFTVQNED | |
| HFGKLESKLSVIRNLNGQVLFIDQGNRPLFADMEASPCRDNAPRTIFIISFYKDSQPRGL | 66 |
| AVTISVKCEKISTLSCENKIISFKEMNPPDNIKDTKSDIIFFLRSVPGHDNKMQFESSSYEG | |
| YFLACEKERDLFKLILKKEDELGDRSIMFTVQNED | |
| LFGKLESKLSVIRNLNGQVLFIDQGNRPLFADMTSSPCRSRAPRTIFIISFYKDSQPRGFA | 67 |
| VTISVKCEKISTLSCENKIISFKEMNPPDNIKDTKSDIIFFIRSVPGHDNKIQFESSSYEGYF | |
| LACEKERDLFKLILKKEDELGDRSIMFTVQNED | |
| HFGKLESKLSVIRNLNDQVLFIDQGNRPLFTDMESKPCRDSAPRTIFIISMYKDSQPRGIA | 68 |
| VTISVKCEKISTLSCENKIISFKEMNPPDNIKDTKSDIIFFIRSVPGHDNKFQFESSSYEGYF | |
| LACEKERDLFKLILKKEDELGDRSIMFTVQNED | |
| YFGKLESKLSVIRNLNRQVLFIDQGNRPLFTDMTYKDCRDNAPRTIFIISFYKDSQPRGF | 69 |
| AVTISVKCEKISTLSCENKIISFKEMNPPDNIKDTKSDIIFFIRSVPGADNKIQFESSSYEGY | |
| FLACEKERDLFKLILKKEDELGDRSIMFTVQNED | |
| HFGKLESKLSVIRNLNGQVLFIDQGNRPLFGDMEASPCRDNAPRTIFIISFYKDSQPRGM | 70 |
| AVTISVKCEKISTLSCENKIISFKEMNPPDNIKDTKSDIIFFIRSVPGADNKLQFESSSYEG | |
| YFLACEKERDLFKLILKKEDELGDRSIMFTVQNED | |
| HFGKLESKLSVIRNLNGQVLFIDQGNRPLFTDMTSSDCRDKAPRTIFIISFYKDSQPRGL | 71 |
| AVTISVKCEKISTLSCENKIISFKEMNPPDNIKDTKSDIIFFLRSVPGPDNKFQFESSSYEG | |
| YFLACEKERDLFKLILKKEDELGDRSIMFTVQNED | |
| HFGKLESKLSVIRNLNGQVLFIDQGNRPLFADMESNRCRDSAPRTIFIISMYKDSQPRGF | 72 |
| AVTISVKCEKISTLSCENKIISFKEMNPPDNIKDTKSDIIFFLRSVPGHDNKIQFESSSYEG | |
| YFLACEKERDLFKLILKKEDELGDRSIMFTVQNED | |
| YFGKLESKLSVIRNLNGQVLFIDQGNRPLFTDMTASPCRDNAPRTIFIISFYKDSQPRGL | 73 |
| AVTISVKCEKISTLSCENKIISFKEMNPPDNIKDTKSDIIFFLRSVPGHDNKIQFESSSYEG | |
| YFLACEKERSLFKLILKKEDELGDRSIMFTVQNED | |
| DFGKLESKLSVIRNLNDQVLFIDQGNRPLFADMKSNVCRANAPRTIFIISMYKDSQPRG | 74 |
| MAVTISVKCEKISTLSCENKIISFKEMNPPDNIKDTKSDIIFFIRSVPGPDNKLQFESSSYE | |
| GYFLACEKERDLFKLILKKEDELGDRSIMFTVQNED | |
| HFGKLESKLSVIRNLNGQVLFIDQGNRPLFGDMEASPCRAKAPRTIFIISIYKDSQPRGFA | 75 |
| VTISVKCEKISTLSCENKIISFKEMNPPDNIKDTKSDIIFFLRSVPGHDNKFQFESSSYEGY | |
| FLACEKERSLFKLILKKEDELGDRSIMFTVQNED | |
| HFGKLESKLSVIRNLNGQVLFIDQGNRPLFADMASNRCRANAPRTIFIISMYKDSQPRGF | 76 |
| AVTISVKCEKISTLSCENKIISFKEMNPPDNIKDTKSDIIFFIRSVPGPDNKFQFESSSYEGY | |
| FLACEKERDLFKLILKKEDELGDRSIMFTVQNED | |
| YFGKLESKLSVIRNLNDQVLFIDQGNRPLFADMKAKACRSNAPRTIFIISFYKDSQPRGF | 77 |
| AVTISVKCEKISTLSCENKIISFKEMNPPDNIKDTKSDIIFFLRSVPGADNKIQFESSSYEG | |
| YFLACEKERDLFKLILKKEDELGDRSIMFTVQNED | |
| HFGKLESKLSVIRNLNHQVLFIDQGNRPLFTDMADNACRDNAPRTIFIISFYKDSQPRGL | 78 |
| AVTISVKCEKISTLSCENKIISFKEMNPPDNIKDTKSDIIFFIRSVPGDDNKMQFESSSYEG | |
| YFLACEKERDLFKLILKKEDELGDRSIMFTVQNED | |
| YFGKLESKLSVIRNLNGQVLFIDQGNRPLFTDMKSNLCRSNAPRTIFIISFYKDSQPRGIA | 79 |
| VTISVKCEKISTLSCENKIISFKEMNPPDNIKDTKSDIIFFIRSVPGDDNKIQFESSSYEGYF | |
| LACEKERDLFKLILKKEDELGDRSIMFTVQNED | |
| HFGKLESKLSVIRNLNGQVLFIDQGNRPLFRDMAASHCRDSAPRTIFIISIYKDSQPRGFA | 80 |
| VTISVKCEKISTLSCENKIISFKEMNPPDNIKDTKSDIIFFLRSVPGHDNKIQFESSSYEGYF | |
| LACEKERDLFKLILKKEDELGDRSIMFTVQNED | |
| YFGKLESKLSVIRNLNDQVLFIDQGNRPLFADMASNPCRYKAPRTIFIISMYKDSQPRGL | 81 |
| AVTISVKCEKISTLSCENKIISFKEMNPPDNIKDTKSDIIFFLRSVPGADNKLQFESSSYEG | |
| YFLACEKERDLFKLILKKEDELGDRSIMFTVQNED | |
| HFGKLESKLSVIRNLNGQVLFIDQGNRPLFTDMASNHCRYNAPRTIFIISMYKDSQPRGL | 82 |
| AVTISVKCEKISTLSCENKIISFKEMNPPDNIKDTKSDIIFFLRSVPGADNKIQFESSSYEG | |
| YFLACEKERDLFKLILKKEDELGDRSIMFTVQNED | |
| HFGKLESKLSVIRNLNGQVLFIDQGNRPLFADMTDNPCRSRAPRTIFIISFYKDSQPRGM | 83 |
| AVTISVKCEKISTLSCENKIISFKEMNPPDNIKDTKSDIIFFIRSVPGHDNKFQFESSSYEG | |
| YFLACEKERDLFKLILKKEDELGDRSIMFTVQNED | |
| YFGKLESKLSVIRNLNGQVLFIDQGNRPLFTDMTASHCRSSAPRTIFIISLYKDSQPRGM | 84 |
| AVTISVKCEKISTLSCENKIISFKEMNPPDNIKDTKSDIIFFLRSVPGHDNKFQFESSSYEG | |
| YFLACEKERDLFKLILKKEDELGDRSIMFTVQNED | |
| YFGKLESKLSVIRNLNGQVLFIDQGNRPLFTDMEYRLCRANAPRTIFIISFYKDSHPRGL | 85 |
| AVTISVKCEKISTLSCENKIISFKEMNPPDNIKDTKSDIIFFLRSVPGDDNKLQFESSSYEG | |
| YFLACEKERDLFKLILKKEDELGDRSIMFTVQNED | |
| YFGKLESKLSVIRNLNGQVLFIDQGNRPLFTDMESSLCRDNAPRTIFIISLYKDSQPRGM | 86 |
| AVTISVKCEKISTLSCENKIISFKEMNPPDNIKDTKSDIIFFLRSVPGADNKFQFESSSYEG | |
| YFLACEKERSLFKLILKKEDELGDRSIMFTVQNED | |
| YFGKLESKLSVIRNLNGQVLFIDQGNRPLFKDMEANDCRSSAPRTIFIISIYKDSQPRGLA | 87 |
| VTISVKCEKISTLSCENKIISFKEMNPPDNIKDTKSDIIFFIRSVPGADNKMQFESSSYEGY | |
| FLACEKERDLFKLILKKEDELGDRSIMFTVQNED | |
| DFGKLESKLSVIRNLNDQVLFIDQGNRPLFADMKASACRANAPRTIFIISMYKDSQPRG | 88 |
| LAVTISVKCEKISTLSCENKIISFKEMNPPDNIKDTKSDIIFFLRSVPGHDNKFQFESSSYE | |
| GYFLACEKERDLFKLILKKEDELGDRSIMFTVQNED | |
| YFGKLESKLSVIRNLNGQVLFIDQGNRPLFGDMTAKHCRARAPRTIFIISFYKDSQPRGM | 89 |
| AVTISVKCEKISTLSCENKIISFKEMNPPDNIKDTKSDIIFFIRSVPGADNKFQFESSSYEG | |
| YFLACEKERDLFKLILKKEDELGDRSIMFTVQNED | |
| FFGKFESKLSVIRNLNGQVLFIDQGNRPLFTDMESKDCRDRAPRTIFIISFYKDSQPRGLA | 90 |
| VTISVKCEKISTLSCENKIISFKEMNPPDNIKDTKSDIIFFLRSVPGHDNKLQFESSSYEGY | |
| FLACEKERDLFKLILKKEDELGDRSIMFTVQNED | |
| FFGKLESKLSVIRNLNGQVLFIDQGNRPLFADMASNHCRANAPRTIFIISLYKDSQPRGL | 91 |
| AVTISVKCEKISTLSCENKIISFKEMNPPDNIKDTKSDIIFFIRSVPGHDNKMQFESSSYEG | |
| YFLACEKERDLFKLILKKEDELGDRSIMFTVQNED | |
| YFGKLESKLSVIRNLNGQVLFIDQGNRPLFADMTSKRCRDNAPRTIFIISLYKDSQPRGF | 92 |
| AVTISVKCEKISTLSCENKIISFKEMNPPDNIKDTKSDIIFFIRSVPGHDNKIQFESSSYEGY | |
| FLACEKERDLFKLILKKEDELGDRSIMFTVQNED | |
| LFGKHESKLSVIRNLNGQVLFIDQGNRPLFGDMESSPCRYNAPRTIFIISFYKDSQPRGLA | 93 |
| VTISVKCEKISTLSCENKIISFKEMNPPDNIKDTKSDIIFFIRSVPGHDNKMQFESSSYEGY | |
| FLACEKERDLFKLILKKEDELGDRSIMFTVQNED | |
| YFGKLESKLSVIRNLNAQVLFIDQGNRPLFTDMTASPCRSSAPRTIFIISLYKDSQPRGLA | 94 |
| VTISVKCEKISTLSCENKIISFKEMNPPDNIKDTKSDIIFFLRSVPGPDNKIQFESSSYEGYF | |
| LACEKERDLFKLILKKEDELGDRSIMFTVQNED | |
| YFGKLESKLSVIRNLNGQVLFIDQGNRPLFADMTDSDCRDNAPRTIFIISMYKDSQPRG | 95 |
| MAVTISVKCEKISTLSCENKIISFKEMNPPDNIKDTKSDIIFFLRSVPGHDNKMQFESSSY | |
| EGYFLACEKERDLFKLILKKEDELGDRSIMFTVQNED | |
| YFGKLESKLSVIRNLNGQVLFIDQGNRPLFADMTSSDCRDNAPRTIFIISFYKDSQPRGM | 96 |
| AVTISVKCEKISTLSCENKIISFKEMNPPDNIKDTKSDIIFFLRSVPGHDNKMQFESSSYEG | |
| YFLACEKERDLFKLILKKEDELGDRSIMFTVQNED | |
| YFGKLESKLSVIRNLNGQVLFIDQGNRPLFADMESSDCRDNAPRTIFIISFYKDSQPRGL | 97 |
| AVTISVKCEKISTLSCENKIISFKEMNPPDNIKDTKSDIIFFLRSVPGHDNKMQFESSSYEG | |
| YFLACEKERDLFKLILKKEDELGDRSIMFTVQNED | |

Also useful, in some embodiments, are nucleic acids that encode a D2D polypeptide, including e.g., one of the D2D polypeptides included above. For example, in some instances, the methods of the present disclosure may include administering to a subject in need thereof, a nucleic acid (e.g., a mRNA or a DNA) that encodes the D2D polypeptide. Such nucleic acids may be administered “naked” (e.g., in the case of naked linear or circular DNA or RNA) or encased or encapsulated, e.g., in a viral particle or non-viral vector. Useful viral and non-viral vectors, and useful components thereof, include e.g., adenoviral vectors, AAV vectors, lentiviral vectors, lipid nanoparticle vectors, and the like. In some instances, the methods of the present disclosure may include administering to a subject in need thereof, a cell (such as e.g., a CD8+ T cell) that comprises an introduced nucleic acid (e.g., a mRNA or a DNA) that encodes the D2D polypeptide. Useful cells may be autologous or allogeneic with respect to the subject being treated. In such cells, such introduced nucleic acids may be integrated into the genome of the cell (e.g., through the use of an integrating vector, site directed nucleases, homologous recombination, or any combination thereof or the like) or may be extrachromosomal. Cells engineered to contain a sequence encoding a D2D polypeptide and express the D2D, (constitutively, conditionally or inducibly) may be referred to as “armored with D2D” or a “D2D armored cell”.

### Antibodies as IL-18, DR-18 and IL-18R Agonists and IL-18BP Antagonists.

Antibodies can be designed to act as IL-18, IL-18R agonists and IL-18BP antagonists include antibodies that specifically bind these molecules and modulate their activity in a therapeutically beneficial way. More specifically, IL-18 agonists include antibodies directed against IL-18 that specifically bind IL-18 and enhance binding of IL-18 to IL-18R; antibodies directed against IL-18R that specifically bind IL-18R and stimulate receptor binding of IL-18 without themselves transducing a signal via IL-18R; antibodies directed against IL-18R that specifically bind IL-18R and mimic the function of IL-18 (30), and the like.

IL-18, IL-18R, and IL-18BP, as well as fragments, variants, muteins, derivatives and fusion proteins thereof, as set forth above, can be employed as “immunogens” in producing antibodies that may be used in the diagnosis and inhibition of immunopathology resulting from pathogenic or autoreactive CD4+ T cell activity. In making IL-18 or IL-18R agonists or IL-18BP antagonists in the form of antibodies, when reference is made to such molecules, it is understood to also encompass fragments, variants, muteins, derivatives and fusion proteins thereof.

Each of IL-18, IL-18R, DR-18, and IL-18BP contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding. Epitopes can be identified by any of the methods known in the art. Additionally, epitopes from the polypeptides of the disclosure can be used as research reagents, in assays, and to purify specific binding antibodies from substances such as polyclonal sera or supernatants from cultured hybridomas. Such epitopes or variants thereof can be produced using techniques known in the art such as solid-phase synthesis, chemical or enzymatic cleavage of a polypeptide, or using recombinant DNA technology.

Antibodies to IL-18, IL-18R, DR-18, IL-18BP, can conveniently be generated against a recombinantly produced form of the proteins described above and provided in the respective sequence identifier numbers. IL-18 agonists can be antibodies that include but are not limited to polyclonal antibodies, monoclonal antibodies (mAbs), humanized or chimeric antibodies, single chain antibodies, Fab fragments, F(ab)2 fragments, fragments produced by a Fab expression library, anti-idiotypic (anti-Id) antibodies, single domain antibodies (sdAbs) and epitope-binding fragments of any of the above. Such antibodies can be utilized in methods of inhibiting immunopathology resulting from pathogenic or autoreactive CD4+ T cell signaling.

Both polyclonal and monoclonal antibodies to the target molecules can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kosbor et al., Immunology Today 4:72, 1983; Cole et al., Proc. Natl. Acad. Sci. USA 80:2026, 1983); and the EBV-hybridoma technique (Cole et al., 1985, Monoclonal Antibodies And Cancer Therapy, Alan R. Liss, Inc., pp. 77-96).

Methods of making humanized monoclonal antibodies are well known, and include for example those described in U.S. Pat. No. 5,585,089 (Protein Design: C L Queen et al.; “Humanized Immunoglobulins”), U.S. Pat. No. 5,565,332 (“Production of Chimeric Antibodies-A Combinatorial Approach”), U.S. Pat. No. 5,225,539 (Med Res Council: G P Winter; “Recombinant Altered Antibodies And Methods Of Making Altered Antibodies”), U.S. Pat. No. 5,693,761-762 (Protein Design: C L Queen et al.; “Polynucleotides Encoding Improved Humanized Immunoglobulins”, and “Humanized Immunoglobulins”), and U.S. Pat. No. 5,530,101 (Protein Design: C L Queen et al.; “Humanized Immunoglobulins”), and references cited therein.

Hybridoma cell lines that produce monoclonal antibodies specific for the target molecules are contemplated herein. Such hybridomas can be produced and identified by conventional techniques. For the production of antibodies, various host animals may be immunized by injection the polypeptide of interest or antigenic fragments thereof. Such host animals may include, but are not limited to, horse, goat, sheep, cow, rabbits, mice, and rats, to name a few. Various adjuvants may be used to increase the immunological response. Depending on the host species, such adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. The monoclonal antibodies can be recovered by conventional techniques. Such monoclonal antibodies may be of any immunoglobulin class including IgG, IgM, IgE, IgA, IgD, and any subclass thereof. The hybridoma producing the mAb may be cultivated in vitro or in vivo. Or, the antibody genes can be cloned and optionally otherwise altered, and expressed in another cell line approved for recombinant production of protein pharmaceuticals such as, for example, CHO cells.

Alternatively, libraries of antibody fragments can be screened and used to develop human antibodies through recombinant techniques. Such libraries are commercially available from, for example, Cambridge Antibody Technology (Melbourne, UK), and Morphosys (Munich, Del.). In addition, techniques developed for the production of “chimeric antibodies” (Takeda et al., Nature, 314:452, 1985) by splicing the genes from a mouse antibody molecule of appropriate antigen specificity together with genes from a human antibody molecule of appropriate biological activity can be used.

A chimeric antibody is a molecule in which different portions are derived from different animal species, such as those having a variable region derived from a porcine mAb and a human immunoglobulin constant region. The monoclonal antibodies of the invention also include humanized versions of murine monoclonal antibodies. Such humanized antibodies can be prepared by known techniques and offer the advantage of reduced immunogenicity when the antibodies are administered to humans. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some (preferably virtually all) antibodies produced by the animal upon immunization.

Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Procedures to generate antibodies transgenically can be found in GB 2,272,440, U.S. Pat. Nos. 5,569,825 and 5,545,806 and related patents claiming priority therefrom, all of which are incorporated by reference herein. For use in humans, the antibodies are typically human or humanized; techniques for creating such human antibodies are also known. Transgenic animals for making human antibodies are available from, for example, Medarex Inc. (Princeton, N.J.) Protein Design Labs, Inc. (Fremont, Calif) and Abgenix Inc. (Fremont, Calif.).

Expression of a humanized immunoglobulin sequences in bacterial hosts may be used to select higher affinity humanized immunoglobulin sequences by mutagenizing the CDR regions and producing bacteriophage display libraries which may be screened for humanized immunoglobulin CDR variants which possess high affinity and/or high specificity binding to the target molecules. One potential advantage of such affinity sharpening is the generation of humanized immunoglobulin CDR variants that have improved binding affinity and/or reduced cross-reactivity with molecules other than the molecule to which they were raised. Methods for producing phage display libraries having immunoglobulin variable region sequences are provided in the art (see, e.g., Cesareni, FEBS Lett 307:66, 1992; Swimmer et al., Proc. Natl. Acad. Sci. USA 89:3756, 1992; Gram et al., Proc. Natl. Acad. Sci. USA 89:3576, 1992; Clackson et al., Nature 352:624, 1991; Scott & Smith, Science 249:386, 1990; Garrard et al., Bio/Techniques 9:1373, 1991; which are incorporated herein by reference in their entirety for all purposes. The resultant affinity sharpened CDR variant humanized immunoglobulin sequences are subsequently expressed in a suitable host.

Antibody fragments, which recognize specific epitopes, may be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries may be constructed (Huse et al., Science, 246:1275, 1989) to allow rapid and easy identification of monoclonal Fab fragments with the desired specificity. Techniques described for the production of single chain antibodies (U.S. Pat. No. 4,946,778; Bird, Science 242:423, 1988; Huston et al., Proc. Natl. Acad. Sci. USA 85:5879, 1988; and Ward et al., Nature 334:544, 1989) can also be adapted to produce single chain antibodies against polypeptides of interest. In addition, such antibodies can, in turn, be utilized to generate anti-idiotype antibodies using techniques known to those skilled in the art. (See, e.g., Greenspan & Bona, FASEB J 7(5):437, 1993; and Nissinoff, J. Immunol 147(8):2429, 1991).

### Nucleic Acid-Based IL-18BP Antagonists

In alternative embodiments, nucleic acid-based immunotherapy can be designed to decrease the level of endogenous IL-18BP gene expression, e.g., using antisense or ribozyme approaches to inhibit or prevent translation of IL18BP mRNA transcripts; triple helix approaches to inhibit transcription of IL18BP; or targeted homologous recombination to inactivate or “knock out” the IL18BP gene or its endogenous promoter.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a target mRNA of interest having a complementary polynucleotide sequence. Absolute complementarity, although preferred, is not required. A sequence “complementary” to a portion of RNA, as referred to herein, means a sequence having sufficient complementarity to be able to hybridize with the RNA, thereby forming a stable duplex. Oligonucleotides that are complementary to the 5′ end of the message, e.g., the 5′ untranslated sequence up to and including the AUG initiation codon, should work most efficiently at inhibiting translation. However, oligonucleotides complementary to either the 5′- or 3′-non-translated, non-coding regions of the targeted gene transcript could be used in an antisense approach to inhibit translation of endogenous target proteins. Oligonucleotides complementary to the 5′ untranslated region of the mRNA should include the complement of the AUG start codon. Antisense nucleic acids should be at least six nucleotides in length, and are preferably oligonucleotides ranging from 6 to about 50 nucleotides in length. The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), or hybridization-triggered cleavage agents or intercalating agents (see, e.g., Zon, 1988, Pharm. Res. 5:539-549).

The antisense molecules are delivered to cells, which express a transcript having an IL18BP polynucleotide sequence in vivo by, for example, injecting directly into the tissue or cell derivation site, or by use of modified antisense molecules, designed to target the desired cells (e.g., antisense linked to peptides or antibodies that specifically bind receptors or antigens expressed on the target cell surface) can be administered systemically or to host cells obtained from the subject to be treated. Another approach utilizes a recombinant DNA construct in which the antisense oligonucleotide is placed under the control of a strong pol III or pol II promoter. The use of such a construct to transfect target cells in the subject will result in the transcription of sufficient amounts of single stranded RNAs that will form complementary base pairs with the endogenous IL18BP transcripts and thereby prevent translation of the targeted mRNA. For example, a vector can be introduced in vivo such that it is taken up by a cell and directs the transcription of an antisense RNA. Such a vector can remain episomal or become chromosomally integrated, so long as it can be transcribed to produce the desired antisense RNA. Vectors can be plasmid, viral, or others known in the art, used for replication and expression in mammalian cells.

Ribozyme molecules designed to catalytically cleave mRNA transcripts having an IL18BP polynucleotide sequence prevent translation of the target mRNA (see, e.g., PCT International Publication WO90/11364, published Oct. 4, 1990; U.S. Pat. No. 5,824,519). Ribozymes are RNA molecules possessing the ability to specifically cleave other single-stranded RNA in a manner analogous to DNA restriction endonucleases. A major advantage of this approach is that, because they are sequence-specific, only mRNAs with particular sequences are inactivated. There are two basic types of ribozymes namely, tetrahymena-type (Hasselhoff, Nature, 334:585-591, 1988) and “hammerhead”-type. Tetrahymena-type ribozymes recognize sequences, which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes.

As in the antisense approach, the ribozymes can be composed of modified oligonucleotides (e.g. for improved stability, targeting, and the like). A typical method of delivery involves using a DNA construct “encoding” the ribozyme under the control of a strong constitutive pol III or pol II promoter, so that transfected cells will produce sufficient quantities of the ribozyme to destroy endogenous autoinflammatory mRNA and inhibit translation. Because ribozymes, unlike antisense molecules, are catalytic, a lower intracellular concentration is required for efficiency.

Alternatively, endogenous IL-18BP expression can be reduced by targeting deoxyribonucleotide sequences complementary to the regulatory region of the target gene (i.e., the target gene promoter and/or enhancers) to form triple helical structures that prevent transcription of the target gene (see generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Antisense RNA and DNA, ribozyme, and triple helix molecules described herein may be prepared by any method known in the art for the synthesis of DNA and RNA molecules and include techniques for chemically synthesizing oligodeoxyribonucleotides and oligoribonucleotides such as, for example, solid phase phosphoramidite chemical synthesis, e.g. by use of an automated DNA synthesizer (such as are commercially available from Biosearch, Applied Biosystems, and the like). As examples, phosphorothioate oligonucleotides may be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. U.S.A. 85:7448-7451). Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters.

Alternatively, antisense cDNA constructs that synthesize antisense RNA constitutively or inducibly, depending on the promoter used, can be introduced stably into cell lines. In alternative embodiments IL-18BP expression may be blocked by post-translational gene silencing, such as by double-stranded RNA-induced gene silencing, also known as RNA interference (RNAi). RNA sequences specific for IL-18BP may be modified to provide double-stranded sequences or short hairpin RNAs for therapeutic use.

### Screening for IL-18, IL-18R, and DR-18 Agonists

IL-18, IL-18R and DR-18 agonists can be evaluated using screening assays known in the art, such as high throughput test systems. The assays can be performed in a variety of formats, including protein-protein binding assays, competition binding assays, biochemical screening assays, immunoassays, cell-based assays, etc. For the sake of clarity, the following exemplary assays are described in the context of IL-18, IL-18R, DR-18 and are therefore illustrative and not limiting.

By observing the effect that an IL-18 agonist has on the interaction between IL-18 and IL-18 receptor in various binding assays, on IL-18/IL-18 receptor-mediated activity in functional tests, and in cell based screens, molecules that are potential therapeutics can be identified because they augment the interaction between IL-18 and IL-18 receptor and enhance the immune-protective functions of IL-18. IL-18 agonists that partially or completely enhance IL-18 binding to IL-18 receptor, and hence the activation related to anti-inflammatory activities of IL-18 receptor, can be useful as immunosuppressants or anti-inflammatory agents in to ameliorate symptoms resulting from pathogenic, autoreactive CD4+ T cells.

In another aspect, the screening methods entail incubation of the test compound, the IL-18 protein and cells expressing IL-18 receptor for a suitable time period; determining the level of biological activity of IL-18 on the IL-18 receptor in presence of the test compound; and comparing the level of biological activity with that which occurs in the absence of test compound, wherein a difference in the level of biological activity indicates that the test compound affects the biological activity of the IL-18/IL-18 receptor complex. Biological activity of IL-18 on the IL-18 receptor can be assayed in any number of ways, for example but not limited to, determining the phosphorylation state of intracellular proteins (i.e., activation of the IL-18 receptor by IL-18); determining the production of proinflammatory factors, such as Interferon gamma (IFNg), IL-6, IL-8, monocyte chemoattractant protein-1 and Groa; determining the production of hematopoietic cytokines, such as G-CSF and GM-CSF and IL-8; determining increased expression of IL-10 and TNF, as well as measuring induction of iNOS in macrophages, and determining any immune protective factors produced.

A particular example of an assay for the identification of potential IL-18 agonists is a competitive assay, which combines IL-18 and an IL-18 receptor-specific agonist with IL-receptor under the appropriate conditions for a competitive assay. Either IL-18 or the IL-18 receptor-specific agonist can be labeled so that the binding can be determined, and the effectiveness of the agonist judged. The label allows for detection by direct or indirect means. Direct means include, but are not limited to luminescence, radioactivity, optical or electron density. Indirect means include but are not limited to an enzyme or epitope tag.

Another method by which IL-18 agonists can be identified that inhibit the interaction between IL-18 and IL-18 receptor is the solid phase method, in which IL-18 receptor is bound and placed in a medium with labeled IL-18. The amount of signal produced by the interaction between IL-18 and IL-18 receptor is measured in the presence and in the absence of a test compound. Diminished levels of signal, in comparison to a control, indicate that the test compound inhibited the interaction between IL-18 and IL-18 receptor. Increased levels of signal, in comparison to a control, indicate that the candidate molecule promotes the interaction between IL-18 and IL-18 receptor. In alternative embodiments, IL-18 could be bound and IL-18 receptor labeled. The IL-18, IL-18 receptor agonists and/or IL-18BP antagonists can be directly or indirectly labeled. For example, if the protein is recombinantly produced, one can engineer fusion proteins that can facilitate solubility, labeling, immobilization and/or detection. Fusion proteins which facilitate these processes can include, but are not limited to soluble Ig-tailed fusion proteins and His-tagged proteins. Methods for engineering such soluble Ig-tailed fusion proteins are well known to those of skill in the art. See, for example, U.S. Pat. No. 5,116,964, and the illustrative embodiments described below. Indirect labeling involves the use of a protein, such as a labeled antibody, which specifically binds to a component of the assay. IL-18, IL-18R and DR-18 agonists can be identified and evaluated using cells and/or cell lines derived from isolated neuronal cells, splenocytes, lymph node cells, immune cells, T cells, B cells and spinal cord cells and cell lines may be used to evaluate IL-18, IL-18R and DR-18 agonists or IL-18BP antagonists in any of the suitable assays described herein. Biologically relevant readouts in the cell-based assay may be used to evaluate potential modulators, such as cell survival; hypertrophic responses; and/or production of molecules in response to hypoxic, environmental stress or immunopathology.

IL-18 modulators can also be identified using methods that are well suited for high-throughput screening procedures, such as scintillation proximity assays (Udenfriend et al., 1985, Proc Natl Acad Sci USA 82: 8672-8676), yeast two-hybrid or interaction trap assays, homogeneous time-resolved fluorescence methods (Park et al., 1999, Anal Biochem 269: 94-104), fluorescence resonance energy transfer (FRET) methods (Clegg R M, 1995, Curr Opin Biotechnol 6: 103-110), or methods that measure any changes in surface plasmon resonance when a bound polypeptide is exposed to a potential binding partner, using for example a biosensor such as that supplied by Biacore AB (Uppsala, Sweden).

Compounds that can be assayed that may also be IL-18, and IL-18R agonists include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co. (Trevillett, Cornwall, UK), MDS Panlabs (Bothell, Wash.), Pharmacopeia (Princeton, N.J,), and Trega (San Diego, Calif). Preferred small organic molecules for screening using these assays are usually less than 10K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2): 193-198). Compounds including natural products, inorganic chemicals, and biologically active materials such as proteins and toxins can also be assayed using these methods to identify molecules having IL-18, IL-18R, an DR-18 protective immune cell activity. In turn these molecules can be manipulated by IL-18 agonists or mimetics to enhance these activities in target cells. For example, IL-18, IL-18R and DR-18 agonists can be administered to a cell or group of cells to increase IL-18:IL-18R, binding and thereby increase therapeutically beneficial cellular communication, cell stimulation, or activity in the target cells. In such an assay, one would determine a rate of communication or cell stimulation in the presence of the IL-18:IL-18R binding and then determine if such communication or cell stimulation is altered in the presence of the agonist. Exemplary assays for this aspect of the invention include cytokine secretion assays, T-cell co-stimulation assays, and mixed lymphocyte reactions involving antigen presenting cells and T cells. These assays are well known to those skilled in the art.

IL-18 agonists may regulate cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of the agonists of interest may be evidenced by any one of a number of factor-dependent cell proliferation assays for cell lines including, without limitation, NFκB, 32D, DA2, DA1G, T10, B9, B9/11, BaF3, MC9/G, M+ (preB M+), 2E8, RBS, DA1, 123, T1165, HT2, CTLL2, TF-1, Mole and CMK.

The activity of IL-18: IL-18R binding may, among other means, be measured by the following methods:

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., PNAS USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7. Immunologic studies in humans); Takai et al., J. Immunol. 137: 3494-3500, 1986; Bertagnolli et al., J. Immunol. 145: 1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994, Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173: 1205-1211, 1991; Moreau et al., Nature 336:690-692, 1988; Greenberger et al., Proc Natl Acad Sci. USA 80: 2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83: 1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology; Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., PNAS USA 77: 6091-6095, 1980; Weinberger et al., Eur. J. Immun. 11:405-411, 1981; Takai et al., J. Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., PNAS USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., PNAS USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144: 3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol 137:3494-3500, 1986; Takai et al., J. Immunol 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., PNAS USA 88:7548-7551, 1991.

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for cell movement and adhesion include, without limitation, those described in: Current Protocols in Immunology Coligan et al. eds, Greene Publishing Associates and Wiley-Interscience (Chapter 6.12, Measurement of alpha and beta chemokines 6.12.1-6.12.28); Taub et al. J. Clin. Invest. 95:1370-1376, 1995; Lind et al. APMIS 103:140-146, 1995; Muller et al Eur. J. Immunol. 25: 1744-1748; Gruber et al. J Immunol. 152:5860-5867, 1994; Johnston et al. J Immunol. 153: 1762-1768, 1994 Assays for hemostatic and thrombolytic activity include, without limitation, those described in: Linet et al., J. Clin. Pharmacol. 26:131-140, 1986; Burdick et al., Thrombosis Res. 45:413-419,1987; Humphrey et al., Fibrinolysis 5:71-79 (1991); Schaub, Prostaglandins 35:467-474, 1988.

### Therapeutic Compositions and Administration Thereof

This disclosure provides compounds, compositions, and methods for treating a subject, preferably a human patient, who is suffering from immunopathology resulting from pathogenic or autoreactive CD4+ T cell disease symptoms. The terms “treat”, “treating”, and “treatment” used herein includes curative, preventative (e.g., prophylactic) and palliative or ameliorative treatment. Modulating therapeutic compositions of IL-18 agonist or mimetics may be administered before, during, and/or after the presentation of symptoms. For therapeutic use, a soluble IL-18, IL-18R and DR-18 modulator is administered to a subject for treatment in a manner appropriate to the indication.

Embodiments of the present disclosure include therapeutic compositions (also referred to as pharmaceutical compositions) comprising one or more soluble IL-18, IL-18R, DR-18 agonists and/or one or more IL-18BP antagonist. A “therapeutic composition,” as used herein, comprises one or more soluble IL-18, IL-18R, DR-18, or IL-18BP modulators and a pharmaceutically acceptable diluent, preservative, solubilizer, emulsifier, adjuvant and/or carrier. As used herein, the terms “pharmaceutically” acceptable and “physiologically” acceptable are used interchangeably. The term “pharmaceutically acceptable” means a non-toxic material that does not interfere with the effectiveness of the biological activity of the active ingredient(s).

Embodiments include therapeutic compositions (also referred to as pharmaceutical compositions) comprising one or more soluble IL-18, IL-18R, DR-18 agonists. A “therapeutic composition,” as used herein, comprises one or more soluble IL-18, IL-18R, and DR-18 modulators and a pharmaceutically acceptable diluent, preservative, solubilizer, emulsifier, adjuvant and/or carrier. As used herein, the terms “pharmaceutically” acceptable and “physiologically” acceptable are used interchangeably. The term “pharmaceutically acceptable” means a non-toxic material that does not interfere with the effectiveness of the biological activity of the active ingredient(s).

Therefore, in certain aspects, the therapeutic compositions comprise one or more of the modulators described in the sections above: e.g., soluble receptor molecules, as well as biologically active fragments, muteins, variants, derivatives, fusions, etc. thereof, antibodies, fusion proteins and/or peptibodies directed against one or more of the following: IL-18, IL-18R, DR-18, IL-18BP; small molecules, such as peptidomimetics, mimotopes and the like, that enhance the interaction between IL-18 and IL-18R; antisense oligonucleotides that specifically target and hybridize to the mRNA of endogenous IL-18BP to inhibit or prevent translation of IL-18BP mRNA transcripts; and RNA-interference molecules tailored to silence expression of IL-18BP. Preferred peptide mimetics comprising active fragments of the proteins listed above that can inhibit or augment the activities of the protein. Peptide mimetics can be 10, 20, 20, 40 or 50 amino acids in length and can optionally be modified to increase bioavailability upon administration to a subject.

Physiologically acceptable carriers, excipients or diluents are nontoxic to recipients at the dosages and concentrations employed. Ordinarily, preparing such compositions entails combining the modulators of interest with buffers, antioxidants such as ascorbic acid, low molecular weight polypeptides (such as those having fewer than 10 amino acids), proteins, amino acids, carbohydrates such as glucose, sucrose or dextrins, chelating agents such as EDTA, glutathione and other stabilizers and excipients. Neutral buffered saline or saline mixed with conspecific serum albumin are exemplary appropriate diluents. The agonists and antagonists preferably are formulated as a lyophilizate using appropriate excipient solutions (e.g., sucrose) as diluents in certain embodiments. Appropriate dosages can be determined in standard dosing trials, and may vary according to the chosen route of administration. In accordance with appropriate industry standards, preservatives may also be added, such as benzyl alcohol. The amount and frequency of administration will depend, of course, on such factors as the nature and severity of the indication being treated, the desired response, the age and condition of the patient, and so forth.

In one embodiment, sustained-release forms of soluble IL-18, IL-18R, DR-18, agonists or IL-18BP antagonists described herein, are used. Sustained-release forms suitable for use in the disclosed methods include but are not limited to, use of molecules that are encapsulated in a slowly dissolving biocompatible polymer, admixed with such a polymer, and or encased in a biocompatible semi-permeable implant. In addition, the modulator molecules may be conjugated with polyethylene glycol (pegylated) to prolong its serum half-life or to enhance protein delivery (as described in detail above).

One type of sustained release technology that may be used in administering soluble target modulator therapeutic compositions is that utilizing hydrogel materials, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59, 1994) and to prevent thrombosis and vessel narrowing following vascular injury (Hill-West et al., Proc. Natl. Acad. Sci. USA 91:5967, 1994). Polypeptides can be incorporated into such hydrogels to provide sustained, localized release of active agents (West and Hubbel, Reactive Polymers 25:139, 1995; Hill-West et al., J. Surg. Res. 58:759; 1995). The sustained, localized release of IL-18 agonist or mimetic for example, when incorporated into hydrogels would be amplified by the long half-life of IL-18.

Therapeutic compositions may be for administration for injection, or for oral, pulmonary, nasal, transdermal or other forms of administration. In general, the invention encompasses therapeutic compositions comprising effective amounts one or more of the agonists and antagonists of the invention individually or together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference in their entirety. The compositions may be prepared in liquid form, or may be in dried powder, such as lyophilized form. Implantable sustained release formulations are also contemplated, as are transdermal formulations.

Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference in its entirety. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556).

A description of possible solid dosage forms for the therapeutic is given in Chapter 10 of Marshall, K., Modern Pharmaceutics (1979), edited by G. S. Banker and C. T. Rhodes, herein incorporated by reference in its entirety. In general, the formulation will include one or more IL-18, IL-18R, DR-18, and IL-18BP modulators and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, N.Y., pp. 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties.

For oral delivery dosage forms, it is also possible to use a salt of a modified aliphatic amino acid, such as sodium N-(8-[2-hydroxybenzoyl]amino)caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell. Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000. Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios. Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms; e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. J. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (al-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor a) and Platz et al., U.S. Pat. No. 5,284,656 (granulocyte colony stimulating factor).

In certain approaches, a wide range of mechanical devices designed for pulmonary delivery of therapeutic products are employed, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Also, the use of liposomes, microcapsules or microspheres, inclusion complexes, or other types of carriers is contemplated.

Nasal delivery of the inventive compounds is also contemplated. Nasal delivery allows the passage of the protein to the blood stream directly after administering the therapeutic compound to the nose, without the necessity for deposition of the product in the lung. Formulations for nasal delivery include those with dextran or cyclodextran. Delivery via transport across other mucous membranes is also contemplated.

One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient.

Initially, the attending physician will administer low doses of the inventive compounds of the present invention and observe the patient's response. Larger doses of the compounds of the present invention can be administered until the optimal therapeutic effect is obtained for the patient, and at that point the dosage is not increased further. It is contemplated that the various therapeutic compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, the inventive therapeutic compounds are administered one time per week to treat the various medical disorders disclosed herein, in another embodiment is administered at least two times per week, and in another embodiment is administered at least three times per week. If injected, the effective amount of therapeutic compounds per adult dose ranges from 1-20 mg/m2, and preferably is about 5-12 mg/m2. Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing the compounds at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg one to three times per week over a period of at least three weeks, or a dose of 50 mg one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of the therapeutic compounds administered by subcutaneous injection one or more times per week. If the compound is in the form of an antibody, a preferred dose range is 0.1 to 20 mg/kg, and more preferably is 1-10 mg/kg.

Another embodiment of a dose range is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

“Concurrent administration” encompasses simultaneous or sequential treatment with the components of the combination, as well as regimens in which the drugs are alternated, or wherein one component is administered long-term and the other(s) are administered intermittently. Components can be administered in the same or in separate compositions, and by the same or different routes of administration.

Examples of other drugs or therapeutic compositions that may be used in combination with inventive compounds of the invention alone or in combination include: analgesic agents, disease-modifying anti-rheumatic drugs (DMARDs), non-steroidal anti-inflammatory drugs (NSAIDs), and any immune and/or inflammatory modulators. Non-steroidal anti-inflammatories may include, but are not limited to: salicylic acid (aspirin); ibuprofen; indomethacin; celecoxib; rituxin, rofecoxib; ketorolac; nambumetone; piroxicam; naproxen; oxaprozin; sulindac; ketoprofen; diclofenac; other COX-1 and/or COX-2 inhibitors, salicylic acid derivatives, propionic acid derivatives, acetic acid derivatives, fumaric acid derivatives, carboxylic acid derivatives, butyric acid derivatives, oxicams, pyrazoles and pyrazolones, including newly developed anti-inflammatories.

Therapeutic compositions of this invention may be administered with one or more of the following: modulators of other members of the TNF/TNF receptor family, including TNF antagonists, such as etanercept (Enbrel™), sTNF-RI, onercept, D2E7, and Remicade™; IL-1 inhibitors, including IL-1ra molecules such as anakinra and more recently discovered IL-1ra-like molecules such as IL-1Hy1 and IL-1Hy2; IL-1 “trap” molecules as described in U.S. Pat. No. 5,844,099; IL-1 antibodies; solubilized IL-1 receptor, and the like; IL-6 inhibitors (e.g., antibodies to IL-6); IL-8 inhibitors (e.g., antibodies to IL-8); Interleukin-1 converting enzyme (ICE) modulators; insulin-like growth factors (IGF-1, IGF-2) and modulators thereof; transforming growth factor-β (TGF-β), TGF-β family members, and TGF-β modulators; fibroblast growth factors FGF-1 to FGF-10, and FGF modulators; COX-2 inhibitors, such as Celebrex™ and Vioxx™; prostaglandin analogs (e.g., E series prostaglandins); matrix metalloproteinase (MMP) modulators; nitric oxide synthase (NOS) modulators, including modulators of inducible NOS; modulators of glucocorticoid receptor; modulators of glutamate receptor; modulators of lipopolysaccharide (LPS) levels; anti-cancer agents, including inhibitors of oncogenes (e.g., fos, jun) and interferons; noradrenaline and modulators and mimetics thereof.

Additional embodiments of compositions that can be administered concurrently with the pharmaceutical compositions of the invention are: cytokines, lymphokines, or other hematopoietic factors such as M-CSF, GM-CSF, Fit3-Ligand, TNF, IL-1, IL-2, IL-3, IL4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin, or inhibitors or antagonists of any of these factors. The pharmaceutical composition can further contain other agents which either enhance the activity of the polypeptide or complement its activity or use in treatment. Such additional factors and/or agents may be included in the pharmaceutical composition to produce a synergistic effect with polypeptide of the invention, or to minimize side effects. Conversely, the inventive compounds may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

Further embodiments of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

The invention having been described, the following examples are offered by way of illustration, and not limitation.

The following materials and methods are provided to facilitate the practice of the present disclosure.

### Mice:

All mice were kept in a specific-pathogen free facility and all experiments performed under a protocol approved by the Children's Hospital of Philadelphia Institutional Animal Care and Use Committee. WT and C57BL/6N-Il18 bp^{tm1.1(KoMP)Vleg (}1118 bp) mice were obtained via the Knock-out Mouse Project (KOMP, UC San Diego https://mmrrc.ucdavis.edu/mta/KOMP-MTA.pdf). IL-18 transgenic mice (Il18tg) were a gift from T. Hoshino, Kurume University¹⁸. 2D2 mice were purchased from the Jackson Laboratory. All 2D2 mice were screened using flow cytometric analysis of PBMCs using specific antibodies to Vα3.2 and Vβ11 (Thermofisher Scientific). All mice used in experiments were males, 6-9 weeks of age at the initiation of the studies. All animal procedures were performed in accordance with approved university protocols.

### Experimental Autoimmune Encephalomyelitis (EAE):

EAE was induced according to a previously established protocol³¹. Briefly, mice were immunized subcutaneously in four sites on the back with a total of 200 μg MOG (35-55) peptide (Biosynthesis) emulsified with CFA with M. tuberculosis strain H37Ra (DIFCO Laboratories). Mice were injected with 200 ng pertussis toxin (List Biological Laboratories) intraperitoneally on days 0 and 2. Mice were assessed daily and scored as follows: 1, flaccid tail; 2, impaired righting reflex and hindlimb weakness; 3, complete hindlimb paralysis; 4, hindlimb paralysis with partial forelimb paralysis; or 5, moribund.

### Rm-IL-18 Treatment:

WT mice were administered recombinant murine IL-18 (MBL, Nagoya, Japan, 0.2 ug/injection as in Kinoshita et al.³²) or PBS in three intraperitoneal injections (i.p.) during the early induction phase of EAE (at days 0, 2, and 4) and during the late effector phase of EAE (at days 9, 11, and 13).

### Flow Cytometry:

Single-cell suspensions were obtained from spleen, lymph node, and spinal cord tissues by mechanical isolation adapted from previously published protocols^{33,34} For spinal cord immune cells, mice were euthanized, perfused with saline, and spinal cords were removed using hydraulic extrusion. Spinal cords were minced using surgical scissors and digested for 30 minutes in a 37° C. incubator in a Collagenase D/DNAse I mixture. Single cell suspensions were then homogenized using an 18G needle and passed through a 70 uM filter. Immune cells were then obtained using a Percoll gradient (30%/60%) centrifugation and collected from the interphase. Cells were washed and stained with antibodies conjugated to fluorochrome dyes. Flow cytometry was performed on a Beckman Coulter CytoFLEX LX and analyzed using FlowJo.

Antibodies used for this analysis include Zombie UV (BioLegend), Tcrb in PE-Cy7 (Tonbo Biosciences), CD8 in AF700 (BD), CD4 in APC Cy-7 (Invitrogen), IFNg in BV488 (BD), IL-17a in PerCP Cy 5.5 (Invitrogen), RORgT in PE (Invitrogen), T-bet in BV421 (Invitrogen), FOXP3 in FITC (Invitrogen), and FC Block (BioLegend).

The following examples are provided to illustrate certain embodiments of the subject matter described. They are not intended to limit the invention in any way.

### Example I

We sought to determine whether or not free or chronic excess of IL-18 would exacerbate or ameliorate the clinical phenotypes of an EAE model. Subcutaneous immunization with 200 μg of MOG₃₅₋₅₅ and two injections of 100 ng of pertussis toxin on days 0 and 2 in mice that no not express IL-18BP, the decoy receptor for IL-18, (i.e., IL-18BP knockout mice, Il18 bp−/−) and mice that overexpress IL-18 (i.e., mice carrying an IL-18 transgene, Il18tg¹⁸) resulted in little to no clinical paralysis in these mice (FIG. 1B and FIG. 1C). To elucidate the importance of timing on the effect of IL-18 in this context, we sought to block IL-18 signaling at either EAE initiation or after establishment of disease. We proceeded to inject wildtype B6 mice with recombinant mouse IL-18 (rmIL-18) at the induction phase (Days 0, 2, and 4), or during the effector phase (Days 9, 11, 13). Interestingly, we found that early addition of rmIL-18 resulted in a delay of paralysis (FIG. 1D). We hypothesized that inhibition of rmIL-18 by endogenous IL-18BP would prevent the effects of rmIL-18 administration in the early phase. To more fully address the effects of exogenous IL-18 signaling, we treated WT mice with either PBS or mouse IL-18BP “decoy resistant” IL-18 (also known as mouse “DR-18”; see e.g., Zhou et al.¹⁴) and found strong protection from EAE with mouse DR-18 administration (FIG. 1E) without signs of hyperinflammation. To better evaluate the impact of timing of IL-18 signaling on inhibiting EAE, we administered a IL-18 receptor blocking antibody to wildtype and Il18 bp−/− mice at various timepoints, including early in disease, throughout the experiment, or later in the course. Early and continuous IL-18 signaling blockade resulted in loss of protection, but so too did blocking IL-18 signaling after the onset of clinical symptoms (FIG. 1F).

Through our experiments, to our surprise, we observed a relative increase in CD8 T-cells in the spinal cords of Il18 bp−/− mice or mice administered DR-18, but not control mice without excess IL-18 (FIG. 2A and FIG. 2B). This relative increase was largely due to a diminution of conventional, but not FoxP3+, CD4 T-cells. 2D2 transgenic mice, which harbor a transgene encoding an MHCII-restricted T-cell Receptor (TCR) specific for a MOG₃₅₋₅₅ peptide, have very few CD8 T cells due to allelic exclusion, but a great abundance of autoreactive, MOG-specific CD4 T cells. We then sought to determine whether or not crossing these 2D2 transgenic mice to Il18 bp−/− mice would make Il18 bp−/− susceptible to clinical paralysis in EAE. Surprisingly, the loss of CD8 T cells and expansion of MOG specific CD4 T cells abrogated the protection of Il18 bp−/− mice from EAE (FIG. 2C).

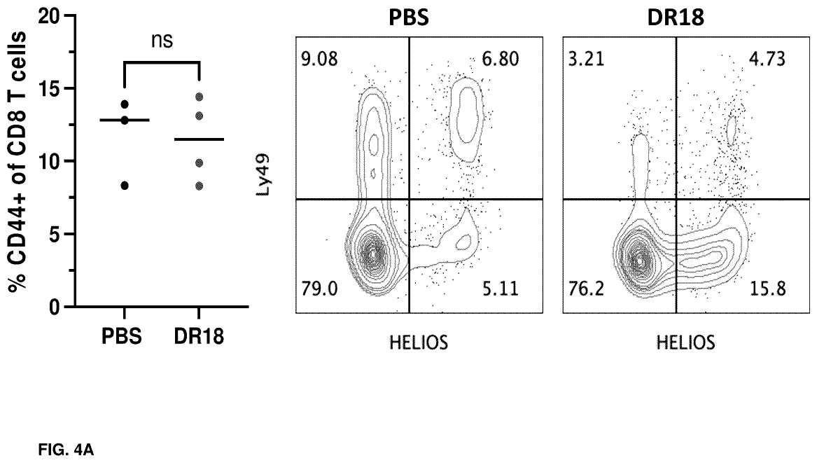

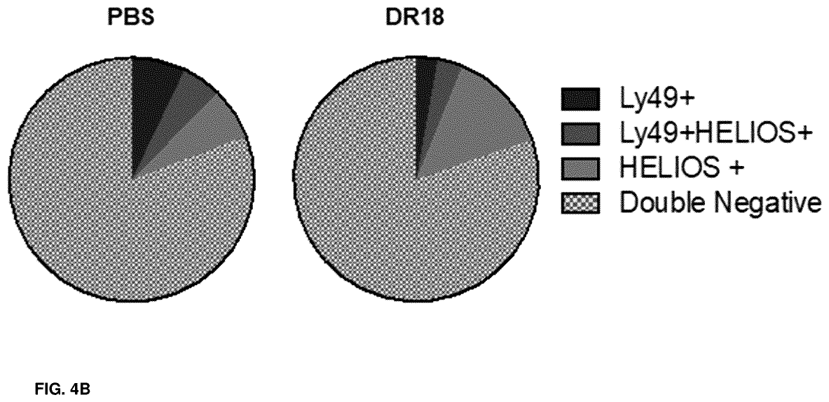

Having shown the beneficial effects of 11-18 in EAE in a variety of systems, and its dependence on the IL-18 receptor, to better characterize what cell types were responding to IL-18 and thereby initiating the protective response we used several transgenic mice. Il18tg mice specifically lacking the Il18r1 gene in T-cells were no longer protected from EAE, and the proportion of the spinal cord cells that were CD8+ was no longer elevated, showing that both protection and CD8 T-cell abundance in the CNS required T-cell IL-18 signaling (FIG. 3A-B). This protection could have been mediated by FoxP3+ Treg responses to IL-18, but deletion of Il18r1 specifically from these cells did not affect protection in Il18tg mice. Finally, to further refine the IL-18 responsive cell type, we induced EAE in Il18tg mice who, upon tamoxifen treatment, deleted the Il18r1 gene only in mature CD8 T-cells. The temporal loss of Il18r1 only in mature CD8 T-cells was sufficient to abrogate the protective effect of IL-18 in EAE, proving that CD8 T-cell responses to IL-18 were required. Though spinal cord CD8 T-cells were equally activated (CD44+) in mice treated with PBS or DR-18, the latter showed greater expression of Helios and less Ly49 (FIG. 4A-B). Ly49 expression has been proposed as a marker of CD8Treg—although high expression of Ly49 may impair CD8Treg function¹². Helios is a transcription factor typically associated with CD4Treg, but in models of autoimmunity Helios may be important for CD8Treg function³⁵.

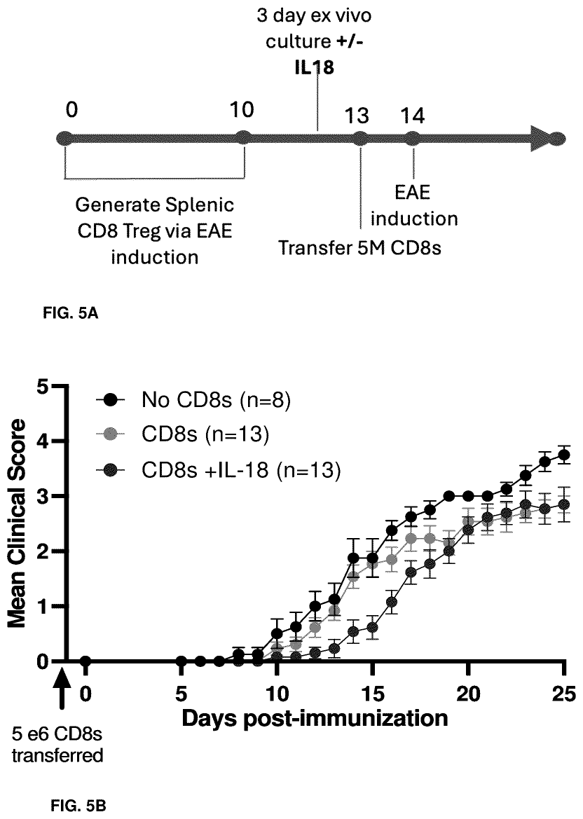

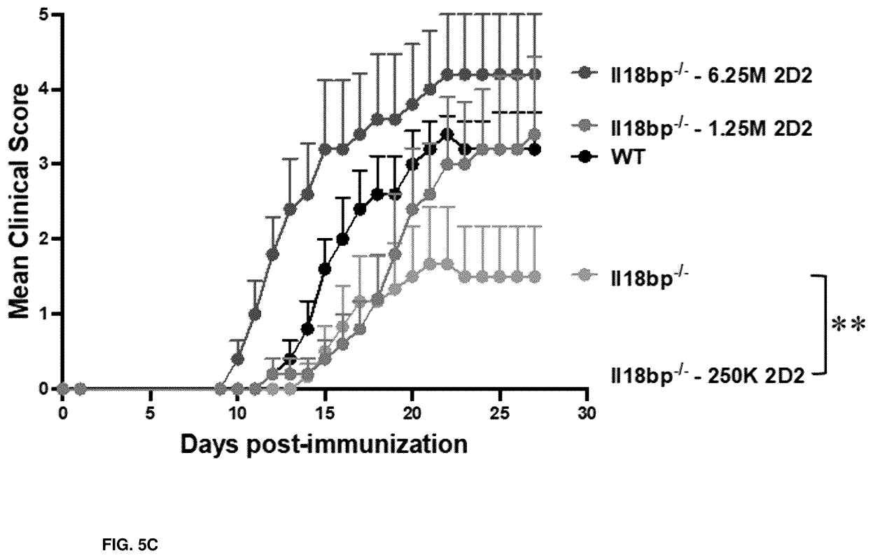

To better understand the role of IL-18 on CD8Treg, we recovered splenic CD8 T-cells from WT EAE-immunized mice at day 10 post-immunization, cultured splenocytes ex vivo with or without IL-18 for 3 days, and then purified CD8 T-cells for adoptive transfer into newly-immunized WT mice (FIG. 5A). Transfer of IL-18-conditioned CD8 T-cells delayed the onset of EAE by a few days, but did not result in profound protection (FIG. 5B). This suggested the IL-18-conditioned CD8Treg may be more efficacious, but likely needed ongoing IL-18 stimulation. CD8Treg are postulated to protect from autoimmunity by acting specifically on autoreactive CD4 T-cells (CD4Tauto). In preparation for determining the fate of transferred 2D2 CD4Tauto, we found that it took a remarkably high number of transferred 2D2 cells to overcome protection in Il18 bp−/− mice. Interestingly, whereas transfer for 1.25 to 6 million 2D2 cells overcame protection, transfer of 250,000 2D2 cells enhanced the already impressive protection in Il18 bp−/− mice (FIG. 5C). One interpretation of this result is that low numbers of 2D2 cells transferred at the time of EAE initiation provided an important antigen presenting cell specifically to IL-18-hungry CD8Tregs.

### Example II

### IL-18 Averts CNS Autoimmunity by Preferentially Activating Protective CD8 Over Autoreactive CD4 T-Cells

Chronic innate immune activation poses a challenge to maintaining self-tolerance, as demonstrated by many autoinflammatory diseases' propensity for autoimmunity. As such, autoinflammation without autoimmune susceptibility, as observed in “inflammasomopathies” with excessive IL-18, suggests active regulation. Experimental Autoimmune Encephalomyelitis (EAE) is a model of autoimmune CNS pathology driven by myelin-reactive, CNS-infiltrating, IL-18-responsive CD4 T-cells (CD4Tₐᵤₜₒ). Contrary to expectations, excess IL-18 (both in Il18-transgenic and Il18 bp^{KO} mice) conferred profound protection. IL-18 did not impair CD4Tₐᵤₜₒ priming or early expansion, but instead selectively limited the later accumulation of cytokine- and integrin-expressing CD4Tₐᵤₜₒ in the periphery and CNS. IL-18's protection required neither signaling on Foxp3⁺ CD4 T-cells nor direct inhibition of CD4Tₐᵤₜₒ. Rather, excess IL-18 acted via its canonical receptor specifically on mature CD8 T-cells, driving a CD8T_{effector} phenotype and IFNg-dependent protection from EAE. Therapeutic administration of “Decoy-resistant” IL-18 (DR-18) engaged CD8 T-cells to diminish CD4Tₐᵤₜₒ, block CNS infiltration, and prevent immunopathology even after CD4Tₐᵤₜₒ expansion. These findings reveal a temporal, dominant, protective, and CD8 T-cell-biased role for IL-18 in EAE, representing a therapeutically relevant mechanism by which chronic inflammasome activation opposes autoimmunity.

### Materials and Methods for Example II

### Mice

All animal studies were performed with approval from the Institutional Animal Care and Use Committee (IACUC) of The Children's Hospital of Philadelphia or University of Pittsburgh. C57Bl/6 (664), 2D2 (6912), Prf1^{-/-} (2407), CD4^{cre} (22071) and Foxp3^{yfp-cre} (16959) mice originated from Jackson Laboratories. Il18 bp^{-/-} mice were obtained from the Knockout Mouse Project. Il18tg mice were a gift from Tomoaki Hoshino (Kurume University), Il18r1^{fl/fl} mice were a gift from G. Trinchieri (National Cancer Institute), E8iCre^{ERT2/GFP} mice were a gift from D. Vignali (University of Pittsburgh).

### Experimental Autoimmune Encephalomyelitis Induction

EAE was induced in 8 to 14 week old mice by subcutaneous injection of 200 μL of CFA/MOG³⁵⁻⁵⁵ emulsion on day 0 with 500 μL of pertussis (400-500 ng based on reported potency, i.p, List Biological Labs) on days 0 and 2. Emulsions were prepared by 2-syringe method using equal parts 1 mg/mL mouse MOG³⁵⁻⁵⁵ (Biosynthesis) and 10 mg/mL CFA (made from BD DIFCO IFA with BD DIFCO H37RA mycobacterium). Clinical score was evaluated daily after day 7 using an established scale from 0 to 5 as follows: 0=no symptoms, 1=tail limpness, 2=tail limpness and hind limb weakness (presenting as wobbly gait or impaired righting reflex), 3=hind limb paralysis, 4=complete hind limb paralysis with forelimb weakness, 5=moribund or death.

### In Vivo Treatment

Antibodies: Mice were treated with various monoclonal antibodies and appropriate isotype-control antibodies via i.p. injection as follows: Anti-IFNg antibody (200 μg, XMG1.2, Tonbo Biosciences) on days 1, 4, and 8. Anti-PD-L1 (250 μg, 10F. 9G2, BioXcell) every 3 days from day 0 to 21. Anti-IL-18R1 antibody (500 ug per dose, Clone 9E6, Genentech) at various timepoints indicated in the figure. Anti-CD8a depleting antibody (200 μg, YTS169.4, BioXcell) on days −3, 0, 3, and 6.

Cytokines: Mice were treated with recombinant murine IL-18 (1 μg, i.p, MBL Life Science.) every other day starting at either day 1 or day 9. Mice were treated with DR-18(23) (2 ug, subcutaneous, gift from SIMCHA therapeutics) every 3 days from day 0 to 15 unless otherwise indicated.

Tamoxifen: Mice were injected with tamoxifen (img, i.p, SIGMA) suspended in sunflower oil (SIGMA) on days 4 and 6 post-EAE induction.

### 2D2 T-Cell Isolation and Transfer

Splenocytes were harvested from naïve 2D2; Il18 bp^{KO} mice via mechanical dissociation and CD4 T-cells were isolated by negative magnetic sorting via Mojosort mouse CD4 T-cell isolation kit (Biolegend). In some experiments, 2D2 T-cells were subsequently labeled with Tag-it-Violet proliferation (BioLegend) dye following manufacturers' protocol. Cells were resuspended in PBS (Corning) and adoptively transferred at appropriate concentration via retroorbital injection. 2D2 T-cell survival and expansion was assessed by flow cytometry.

### CD8 T-Cell Isolation and Transfer

WT mice were immunized with MOG³⁵⁻⁵⁵ for EAE induction and spleens were harvested on day 10. Splenocytes were cultured for 3 days with rmIL-2 (10 pg/mL. Peprotech), MOG³⁵⁻⁵⁵ peptide (20 μg/mL, Biosynthesis), +/−IL-18 (50 μg/mL, MBL Life Science). After 72 hours, CD8 T-cells were isolated by magnetic sorting using MojoSort Mouse CD8 T Cell Isolation Kit (Biolegend8). 5×10⁶ CD8 T-cells were transferred to naive WT mice by retro-orbital injection followed by EAE induction 24 hours later.

### Tissue Collection and Cell Isolation

Leukocytes from spleen and draining lymph node were isolated by mechanical dissociation through a 100 μM strainer. Spinal cord mononuclear cell isolation was adapted from a published protocol(93). Briefly, mice were perfused with cold PBS (Corning) by cardiac puncture and spinal cords were extruded into cold RPMI (Cytiva). Cords underwent mechanical and enzymatic digestion (collagenase and DNAse I in RPMI, 370 for 30 min) followed by 30/70% Percoll (Cytiva) density gradient separation and leukocytes were collected from the interphase for further analysis. Whole blood was collected into EDTA tubes and complete blood counts were assessed using a Drew Scientific Hemavet 950. For flow cytometry, whole blood was lysed with ACK lysis buffer (Quality Biological) and filtered through a 100 uM strainer. Serum was isolated from whole blood by centrifugation using standard serum separator tubes. All single-cell suspensions were prepared in PBS with 2% fetal-bovine serum (Atlanta Biologicals) for flow cytometry.

### Flow Cytometry and Analysis

Single cell suspensions were stained with surface antibodies in HBSS (GIBCO) for 30 minutes followed by fixation and intracellular staining using eBioscience FOXP3/Transcription factor staining buffer set (Thermofisher). Samples were acquired using a 5-laser Cytek Aurora spectral cytometry or Beckman Coulter Cytoflex LX cytometer and analyzed on FlowJo v10.10. For leukocyte analysis, cells were defined as follows: splenic myeloid (NK1.1⁺CD11b⁺), NK cells (B220⁻/TCRb⁻/NK1.1⁺), T cells (NK1.1⁻/CD11b⁻/B220⁻/TCRb⁺/CD4⁺ or CD8⁺). T-cells were further divided as follows: CD4T_{conv} (FOXP3⁻), CD4T_{reg} (FOXP3⁺), 2D2 (CD4⁺CD8⁻/TCRVa3.2⁺/TCRVb11⁺; T_{eff} (CD44^{hi}CD62L⁻), central memory T/Tₑₘ (CD44^{hi}/CD62L⁺), and naive T (CD44^{lo}, CD62L⁻). Spinal cord leukocytes were assessed using the same criteria except all leukocytes were initially defined by CD45 expression and myeloid populations were subdivided between CD45^{hi} and CD45^{mid}. For UMAP visualization of day 12 splenic samples, an equal number of WT and Il8 bp^{KO} CD4 T-cells were concatenated from 4 samples per group. UMAP visualization was performed using FlowJo Exchange UMAP plugin (v4.1.1) only on 2D2 T-cells in the concatenated samples.

### Ex Vivo T Cell Stimulation

Cells were plated at 2M/mL in R10 media (RPMI1640; GIBCO, 10% FBS; Atlanta Biologicals) and incubated for 5 hours with PMA/Ionomycin/Brefeldin A (BioLegend, cell activation cocktail with Brefeldin A) or 18 hours with MOG³⁵⁻⁵⁵ peptide or no peptide. For peptide stimulation, Brefeldin A (BioLegend) was added for the final 5 hours of culture followed by intracellular staining as above. For cytokine measurements in supernatant, cells were isolated and plated in the same conditions above for 72 hours with MOG³⁵⁻⁵⁵ without brefeldin A.

### Cytokine Measurements:

IFNg serum levels were assessed by BD OptEIA Mouse IFN-γ ELISA following manufacturers protocol (BD Biosciences) with absorbance measurements using ThermoFisher VarioSkan Lux. All other cytokine and chemokine measurements were assessed using mouse cytometric bead array kits (BD Biosciences) following manufacturers protocol. Samples were analyzed using 3-laser Beckman Coulter CytoFLEX Flow cytometer. All cytokine measurements shown are the average of 2 technical replicates with a baseline offset equal to the lowest standard (3.5 pg/mL in ELISA, 5 pg/mL in CBA)

### Histology:

Mice were perfused with PBS followed by 4% paraformaldehyde (PFA) via cardiac puncture. Spinal cords were gently extruded from the vertebral column using a PBS-filled syringe. Cords were immediately fixed for 48 hours in 4% PFA, processed using Tissue-Tek VIP 6AI Tissue processor, and paraffin embedded in a sagittal orientation. 5 micrometer paraffin-embedded sections were cut, deparaffinized, and stained with hematoxylin and eosin. Inflammatory foci were counted at 100× magnification for the length of intact cord per each section.

Statistical Analysis: Frequentist statistical analyses were performed in GraphPad Prism v10 on representative or pooled experiments as indicated in figure legends. EAE experiments were analyzed by calculating individual area under the curve (AUC) of clinical disease score over time for each mouse followed by the appropriate statistical tests indicated to evaluate significance. Significance represented by number or symbol where ns=not significant, *=p<0.05, **=p<0.01, ***=p<0.001, ****=p<0.0001

### Results

Excess IL-18 Protects Against EAE without Signs of Hyperinflammation

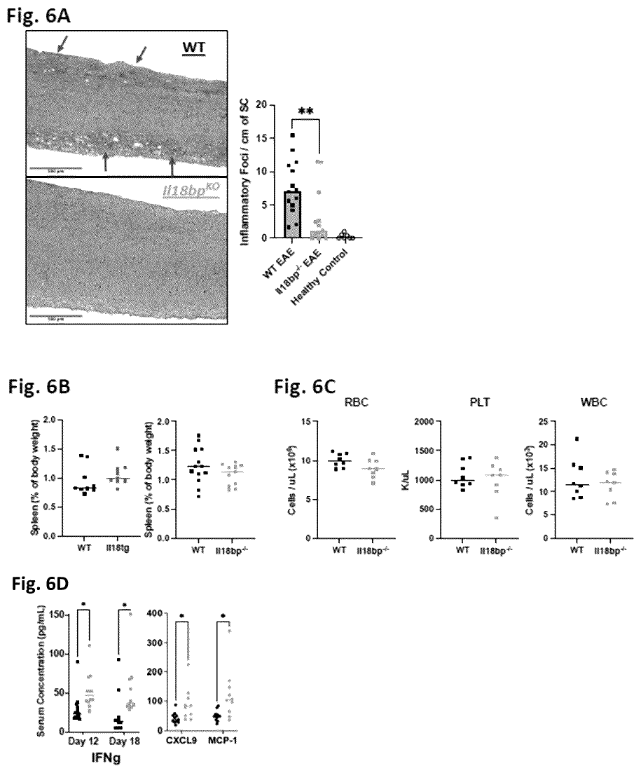

As described in Example 1, following MOG³⁵⁻⁵⁵ EAE induction, WT mice developed the expected progressive, ascending paralysis after about 14 days but, both Il18tg and Il18bpe^{KO} mice were unexpectedly protected from the development and progression of neurologic defects and weight loss. See FIGS. 1B and 1C. Histological examination of Il18bpe^{KO} spinal cords revealed a marked reduction in the number of inflammatory foci relative to WT mice (FIG. 6A). Notably, in Il18bpe^{KO} mice, protection was a direct effect of IL-18, as both IL-18 deletion (Il18^{KO}; Il18bp^{KO}) and IL-18R1 blockade abrogated protection from EAE. Importantly, neither Il18tg nor Il18 bp^{KO} mice showed excessive weight loss, splenomegaly, anemia, thrombocytopenia, or leukopenia indicative of hyperinflammation.

Given the canonical role of IL-18 in amplifying IFNγ, and the complex involvement of IFNγ in EAE pathogenesis, we assessed whether excess IL-18 led to greater levels of IFNγ and related cytokines/chemokines during EAE. This was performed in 118 bp^{KO} mice, as Il18tg mice show slightly elevated serum IFNg levels even without stimulation. Serum from Il18 bp^{KO} mice showed mildly increased IFNg at day 12 and 18, accompanied by increases in the IFNγ-inducible chemokines CXCL9 and MCP-1 at day 12 (FIG. 6D). Additionally, homogenate from draining lymph nodes (dLN) of Il18bp^{KO} mice contained significantly more CXCL9 than WT controls. These data suggest that, although Il18bpe^{KO} mice are protected from EAE pathology, excess IL-18 induces modestly increased IFNg and downstream cytokines/chemokines.

### Excess IL-18 Prevents Accumulation of Activated CD4 T-Cells in the Periphery and CNS

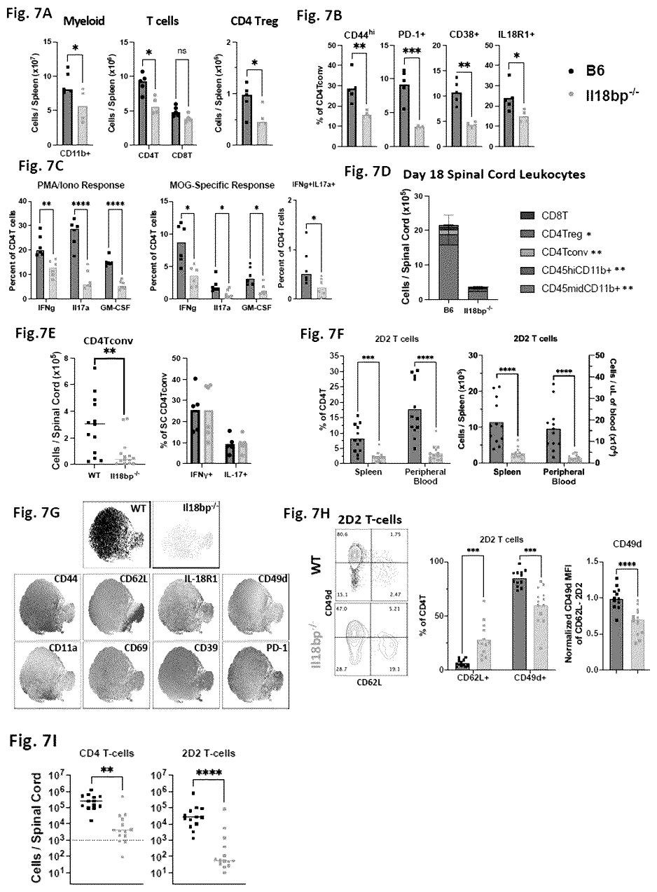

We analyzed peripheral immune cells at day 12, roughly the peak of T cell priming and earliest onset of symptoms. Il18 bp^{KO} mice exhibited a modest decrease in splenic cellularity, primarily driven by fewer myeloid cells (CD11b⁺) and CD4 T-cells, including FoxP3+ regulatory T-cells (CD4T_{reg}) with no major differences in other populations (FIG. 7A). We assessed CD4 T-cell phenotypes and found that Il18 bp^{KO} mice showed a substantial decrease in expression of activation (CD44 and IL-18R1) and inhibitory (PD-1 and CD38) markers (FIG. 7B).

Secretion of IL-17, IFNγ, and GM-CSF have been associated with those autoreactive CD4 T-cells (CD4Tₐᵤₜₒ) necessary for driving EAE pathology. CD4 T-cells from Il18bp^{KO} mice demonstrated decreased ex vivo production of all three cytokines following both non-specific and MOG³⁵⁻⁵⁵ peptide stimulation. This included the IFNg/IL-17 “double positive” population associated with both EAE and MS. Similar trends were seen in peptide-stimulated splenocyte culture supernatants (FIG. 7C). Th17 cells may undergo microbiota-induced “licensing” in the gut before trafficking to the CNS and driving damage in EAE. Nlrc4^{GOF} mice overproduce IL-18 from intestinal epithelia and show type 1 immune activation in the gut but lack systemic free IL-18). Nlrc4^{GOF} mice were not protected from EAE.

Similarly, Il18 bp^{KO} mice had fewer spinal cord leukocytes during periods of CNS involvement (days 18 & 24), including myeloid cells, FOXP3⁻ “conventional” CD4 T-cells (CD4T_{conv}), and to a lesser extent CD4T_{reg} (FIG. 7D). Despite this reduction, those Il18bpe^{KO} CD4T_{conv}, identified in the spinal cord maintained IFNg and IL-17 production (FIG. 7E). Overall, excess IL-18 limited (but did not entirely prevent) the CD4Tₐᵤₜₒ activation and CNS infiltration required for EAE pathology, but it also decreased peripheral and CNS CD4T_{reg}.

To specifically assess the impact of excess IL-18 on CD4Tₐᵤₜₒ, we performed transfer experiments using “2D2” T-cells derived from a mouse with transgenic expression of a MOG³⁵⁻⁵⁵-specific TCR(44). 2D2 CD4 T-cells were transferred into WT and Il18 bp^{KO} mice prior to EAE induction. 5 days later, we found equivalent 2D2 proliferation in peripheral lymphoid organs, suggesting IL-18 does not alter early antigen-presentation or priming despite its ultimate protective effect on EAE. Transfer of up to 250,000 2D2 T-cells prior to EAE induction did not significantly affect disease in either strain. By day 12, WT mice demonstrated substantial expansion and accumulation of 2D2 T-cells in spleens and blood. By contrast, Il18 bp^{KO} mice failed to accumulate 2D2 T-cells, with nearly five-fold fewer cells in both compartments (FIG. 7F.

To better discern differences in transferred 2D2 T-cells, we visualized their surface phenotypes by UMAP (FIG. 7G). Those recovered from WT mice showed surface markers consistent with highly activated effector status (CD44^{hi}, CD62L⁻, IL-18R1⁺) including expression of integrin subunits critical for CD4Tₐᵤₜₒ CNS-infiltration (CD49d and CD11a). By contrast, a smaller proportion recovered from Il18 bp^{KO} mice displayed this same activated effector phenotype, and more cells expressed a “central-memory” phenotype (CD62L⁺, CD49d⁻) (FIGS. 7G-7H). Even among effector 2D2 T-cells (CD44^{hi} CD62L⁻), those from Il18bp^{KO} mice showed lower CD49d expression (FIG. 7H). Both endogenous and 2D2 CD4 T-cells were reduced in spinal cords of Il18 bp^{KO} mice (FIG. 7I). The few 2D2 T-cells detected in Il18bpe^{KO} spinal cords showed no differences in per-cell expression of activation markers, including CD49d. Together, these findings suggest that excess IL-18's effects during the early priming phase of EAE (before day 5) are limited, but at later timepoints excess IL-18 dramatically diminishes the number and activation state of peripheral CD4 T-cells and number of CNS CD4's.

### IL-18-Responsive T-Cells Mediate Protection Against EAE

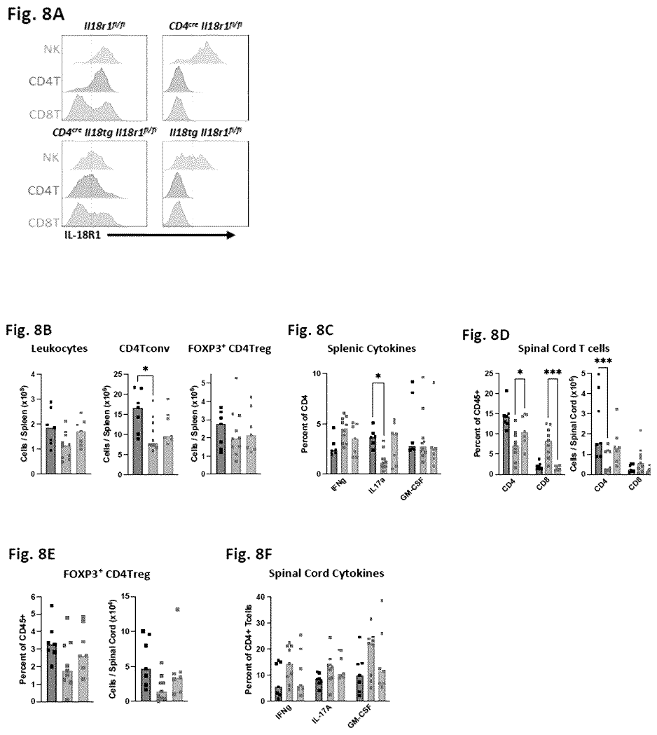

Development and regulation of EAE involves a variety of IL-18 responsive lymphocytes, antigen-presenting, and non-hematopoietic cells. To better understand how IL-18 exerts its protective effects, we generated Il18tg mice lacking the IL18-receptor specifically on T-cells (Il18tg; Il18r1^{DT}). As expected, IL-18R1 was absent on spinal cord and splenic CD4 and CD8 T-cells, but retained on NK cells, from Il18tg; Il18r1^{DT} mice but not Il18tg; Il18r1^{fl/fl} controls lacking Cre-recombinase expression (FIG. 8A). T-cell-specific loss of IL-18R completely abrogated the protective effect of excess IL-18 on EAE score (see FIG. 3A), but did not restore the number of splenic CD4T_{conv} or CD4T_{reg} (FIG. 8B). Intracellular cytokine staining of splenocytes revealed that loss of Il18r1 in T-cells of Il18tg mice restored CD4 T-cell IL-17 production, with minimal effects on IFN7 and GM-CSF production (FIG. 8C). Spinal cord CD4 T-cells, suppressed in control Il18tg mice, were restored to WT levels in Il18tg; Il8r1^{DT} mice (FIG. 8D). We observed similar trends in CNS CD4T_{reg}, and the opposite trend in CNS CD8 T-cells (FIGS. 8D-8E). We did not observe substantial differences in intracellular cytokine staining from CNS CD4 T-cells (FIG. 8F). Overall, these data demonstrate that IL-18 acts directly on a population of T-cells to limit EAE and the accumulation of CD4, but not CD8, T-cells in the CNS.

IL-18 does not Directly Inhibit Autoreactive CD4 T-Cells or Protect Through FOXP3⁺ CD4T_{reg}

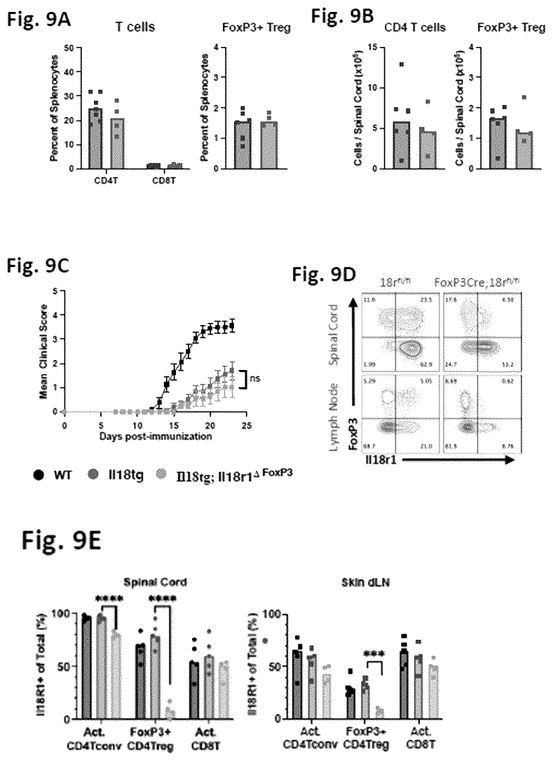

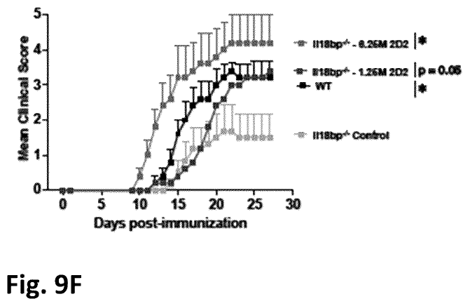

We next asked whether IL-18 directly impaired CD4Tₐᵤₜₒ, limiting their expansion and CNS-homing, or instead augmented protective FOXP3⁺ Tregs during EAE. IL-18 can promote FOXP3⁺ CD4T_{reg}-mediated suppression of inflammatory Th17 cells, but IFNg is thought to promote CD4T_{reg} fragility. To begin testing these possibilities, we generated 2D2; Il8 bp^{KO} mice, combining the autoreactive 2D2 TCR with unopposed IL-18. 2D2 mice are highly enriched for CD4Tₐᵤₜₒ, but also induce functional FOXP3⁺ CD4T_{reg} during EAE(46). With normal IL-18 regulation, 2D2 mice developed more rapid and severe EAE compared to WT mice, as expected. In contrast to the protection observed in Il18 bp^{KO} mice, 2D2; Il18bp^{KO} instead developed more severe EAE than 2D2 controls as shown in FIG. 2C. This observation confirmed that IL-18 was not directly cytotoxic to CD4Tₐᵤₜₒ, and (at least in 2D2-transgenic mice) even enhanced pathogenicity. This paradoxical exacerbation of EAE in 2D2; Il8 bp^{KO} mice was not attributable to defective CD4T_{reg} differentiation, as all mice showed substantial FOXP3⁺ CD4T_{reg} populations in the periphery and spinal cord (FIGS. 9A-9B). Both 2D2 and 2D2; Il8 bp^{KO} contained very few CD8T-cells during EAE (FIG. 9A), as expected given allelic exclusion. To further evaluate the relevance of CD4T_{reg} to IL-18's protection, we examined their phenotypes in WT and Il18 bp^{KO} mice during EAE. A variety of surface markers and transcription factors in CD4T_{reg} showed no significant differences. We generated Il18tg mice lacking IL-18R1 specifically in FOXP3-expressing CD4T_{reg} (Foxp3^{YFP-Cre} Il18r1^{fl/fl} Il18tg). These Il18r1^{ΔFoxp3} mice did not lose protection from EAE (FIG. 9C), despite efficient deletion of Il8r1 specifically in FOXP3⁺ CD4T_{reg} (FIGS. 9D, 9E). Together, these data demonstrate that excess IL-18 does not exert its protective effect directly through FOXP3⁺ CD4T_{reg} or conventional CD4Tₐᵤₜₒ.

Intrigued by the paradoxical exacerbation of EAE in 2D2; Il8 bp^{KO} mice, we wondered whether a surplus of autoreactive 2D2 T-cells would overcome protection in Il18 bp^{KO} mice. Transfer of 250,000 2D2 T-cells did not overcome protection. Il8 bp^{KO} mice who received 1.25 million 2D2 T-cells prior to EAE induction still demonstrated a delay in EAE onset but equivalent peak clinical disease compared to WT. Transfer of 6.25 million 2D2s into Il18 bp^{KO} mice finally overcame protection by excess IL-18 (FIG. 9F). Notably, we derived 2D2 T-cells from the spleens of unstimulated mice, and of these about 4% expressed FOXP3⁺. However, following transferring and EAE induction, <2% of transferred 2D2 T-cells expressed FOXP3 (data not shown). Thus, IL-18's protection was resistant to transfer of >1 million MOG-autoreactive CD4 T-cells, and it did not appear to be mediated directly through CD4Tₐᵤₜₒ or CD4T_{reg}.

Excess IL-18 Activates and Expands Effector CD8 T-Cells, while CD8 Depletion Limits Protection

In models of hyperinflammation, we have previously found that excess IL-18 preferentially activated CD8 over CD4 T-cells. As such, and in light of our findings in EAE, we next considered whether CD8 T-cells were mediating IL-18's protective activity. CD8 T-cells have been shown to play an important regulatory role in EAE and similar models of autoimmunity, but markers used to define a regulatory CD8 T-cell (CD8T_{reg}) population have varied, including general effector/exhaustion markers like PD-1 and CD39, or more specific markers like CD122, Ly49, HELIOS, FOXP3, and/or CD38.

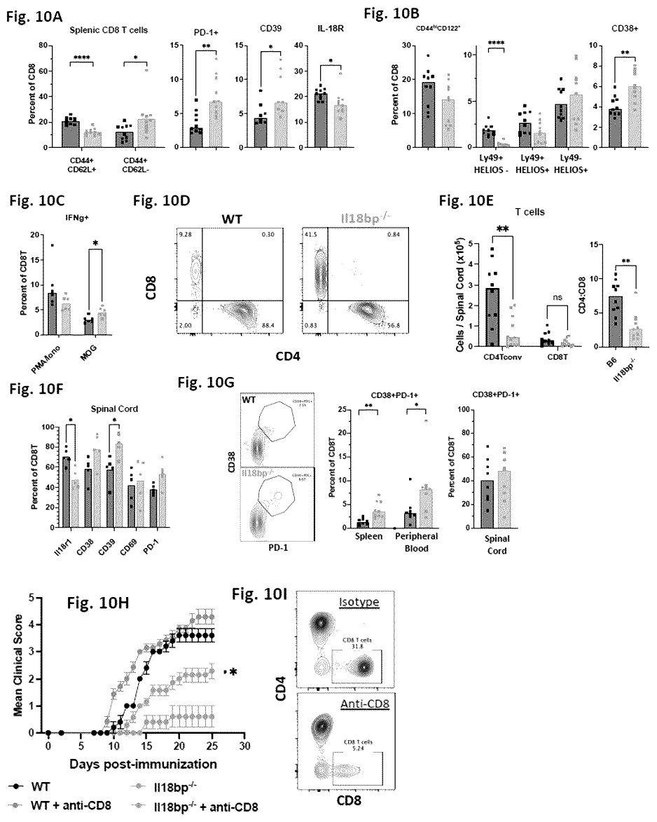

At day 12, splenic CD8 T-cells in Il18bp^{KO} mice showed greater effector (CD44⁺CD62L⁻) than central-memory (Tcm, CD44⁺CD62L⁺) activation, as well as higher expression of CD39 and PD-1 (FIG. 10A-10B). We have previously observed paradoxical IL-18R1 downregulation in Il18tg mice, likely as a regulatory response. This heightened CD8 T-cell activation contrasted with reduced CD4 T-cell activation observed in Il18 bp^{KO} mice (FIG. 7). Expression of other putative CD8T_{reg} surface markers was mixed: CD122 and Ly49 were reduced, FOXP3 expression was negligible in both genotypes, HELIOS (the transcription factor associated with Ly49+ CD8Treg was slightly elevated, and CD38 was increased (FIG. 10B). In some circumstances, protection by CD8 T-cells correlated with IFNg production. Despite IL-18's well-established effect on CD8 T-cell IFNg production, ex vivo IFNg production did not differ dramatically overall, with a trend toward greater PMA-induced IFNg production by WT and greater MOG-peptide-induced IFNg production by Il18bpe^{KO} CD8 T-cells (FIG. 10C). Given our use of genetic models, it remained possible that these differences were unrelated to EAE induction. Without stimulation, CD8 T-cell phenotypes were remarkably similar between WT and Il18bp^{KO} mice, whereas Il18tg mice (reported to have basal activation) showed modest, IL18R1-dependent changes specifically in HELIOS and Ly49 expression. In the CNS, total CD8 T-cells were comparable between genotypes, despite greatly reduced CNS CD4 T-cells in Il18bpe^{KO} mice (FIG. 7D, FIGS. 10D-10E). Differences in surface marker expression were blunted, but otherwise conserved between splenic and CNS CD8 T-cells (FIG. 10F). A population of T-cells (CD44^{hi}CD62L⁻) co-expressing CD38 and PD-1 was expanded in Il18bp^{KO} mice in both spleen and PB at peak disease and seen in the CNS of all mice (FIG. 10G). Thus, CD8 T-cell phenotyping suggests that excess IL-18 drives a program indicative of a highly-activated effector state.

We next examined their relevance by broadly depleting CD8 T-cells beginning at the time of EAE induction. Depleted WT mice showed a trend toward earlier disease onset, but Il18bp^{KO} mice showed a more substantial loss but remained somewhat protected (FIG. 10H). Consistent with prior reports however, depletion of CD8 T-cells was incomplete in both spleens and draining lymph nodes and the remaining CD8T-cells contained a significant effector population (FIG. 10I.

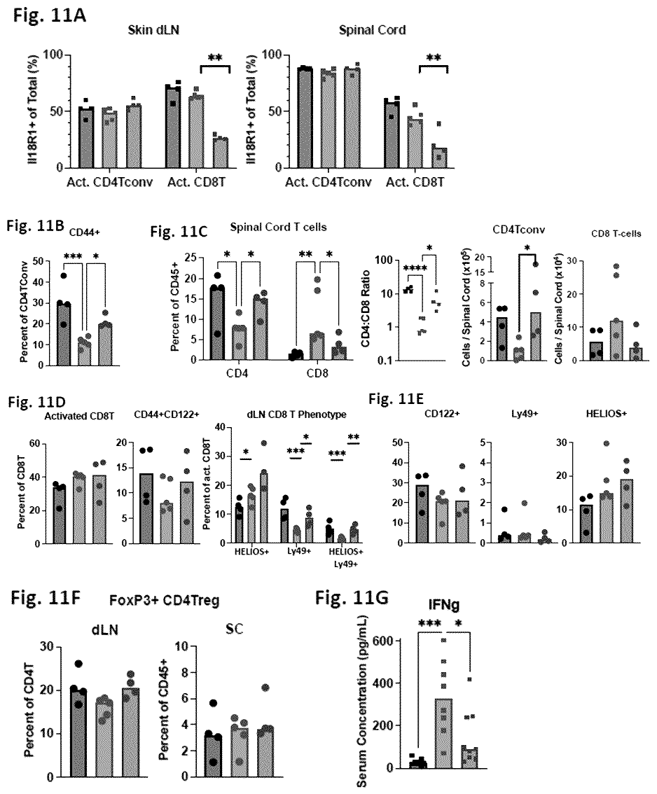

Excess IL-18 protects against EAE via IL-18-responsive CD8 T-cells and IFNγ To determine the extent to which IL-18's protective effect in EAE depended on direct IL-18 sensing by CD8 T-cells, we bred Il18tg mice with tamoxifen-inducible deletion of Il18r1 specifically on mature CD8 T-cells (E8i^{cre/cre}Il18r^{fl/fl}Il18tg, or Il18r1^{ΔCD8}). We induced EAE in Il18tgIl18r1^{ΔCD8} mice (and relevant controls) and, to limit effects on priming and disease penetrance, administered tamoxifen on days 4 & 6 post-EAE induction. Il18tgIl18r1^{ΔCD8} mice lost all protection conferred by IL-18 overexpression as shown in FIG. 3C. As expected, IL-18R expression was lost specifically, but incompletely, on dLN and spinal cord CD8 T-cells of Il18tgIl18r1^{ΔCD8} mice (FIG. 11A. Correspondingly, Il18tgIl18r1^{ΔCD8} mice showed restoration of both dLN CD4T_{conv}, CD44 expression and spinal cord CD4T_{conv}, accumulation (FIGS. 11B-11C). Likewise, the accumulation of CNS CD8 T-cells in Il18tg mice was lost with e8i-specific deletion of Il18r1, significantly altering the CD4:CD8 ratio (FIG. 11C). Il18tgIl18r1^{ΔCD8} mice enabled a more careful dissection of acute versus chronic changes. HELIOS expression by CD8 T-cells was diminished with broad, constitutive Il18r1 deletion (CD4^{Cre}, but remained upregulated in Il18tgIl18r1^{ΔCD8} mice (FIGS. 11D-11E), suggesting HELIOS upregulation is indirect and unnecessary for protection. By contrast, temporal, e8i-specific deletion of Il18r1 was sufficient to prevent downregulation of CD122 and Ly49 (FIG. 11D). As in other systems, we did not observe substantial differences in the phenotype of CNS-infiltrating T-cells, nor in FOXP3+ CD4T_{reg} peripherally or in the CNS (FIGS. 11E-11F). Overall, these experiments suggest a potent, dominant, protective effect of IL-18 signaling specifically through mature CD8 T-cells.

CD8 T-cell IFNγ production was modestly elevated in Il18tg mice and minimally affected in Il18bpe^{KO} mice (FIGS. 8B, and serum IFNγ levels were likewise only modestly elevated (FIGS. 6D & 11I). Nevertheless, given their strong link we next assessed the role of IFNγ in Il18bpe^{KO} mice. Consistent with prior reports, continuous IFNγ neutralization resulted in more severe EAE in WT mice. IFNg-blocked Il18bpe^{KO} mice were not only susceptible to EAE but their disease was comparable to IFNγ-blocked WT mice. IFNγ also appears to be necessary for in vitro, contact-dependent suppression of CD4Tₐᵤₜₒ by CD38⁺ CD8 T-cells, and these data suggest IFNγ plays a crucial role in vivo during IL-18-mediated protection from EAE as well. IFN γ is a strong inducer of PD-L1 on APCs and other cells, and PD-L1 has a known suppressive effect on EAE. However, PD-L1 blockade had no effect on protection in Il18 bp^{KO} mice.

Synthetic, Decoy-Resistant IL-18 (DR-18) Disrupts Autoimmune Effector Activity and Protects from EAE Immunopathology

Targeting specific cytokines has been a transformative therapeutic strategy in dozens of inflammatory diseases. Given profound protection by excess IL-18 in genetic systems, we wondered whether exogenous IL-18 could be effective by itself or as part of immunoregulatory CD8 T-cell therapies. Adapting an established strategy, we cultured CD8 T-cells (from day 10 EAE spleens) with MOG³⁵⁻⁵⁵ peptide+/−IL-18 for 3 days before transferring into WT mice and inducing EAE. CD8 T-cell transfer was not sufficient to prevent EAE, but IL-18-stimulation of CD8 T-cells prior to transfer substantially delayed onset relative to controls (data not shown) suggesting that IL-18's protection required more continuous exposure. However, recombinant murine IL-18 given every other day did not significantly protect WT mice, possibly because we failed to overcome neutralization by endogenous IL-18BP.

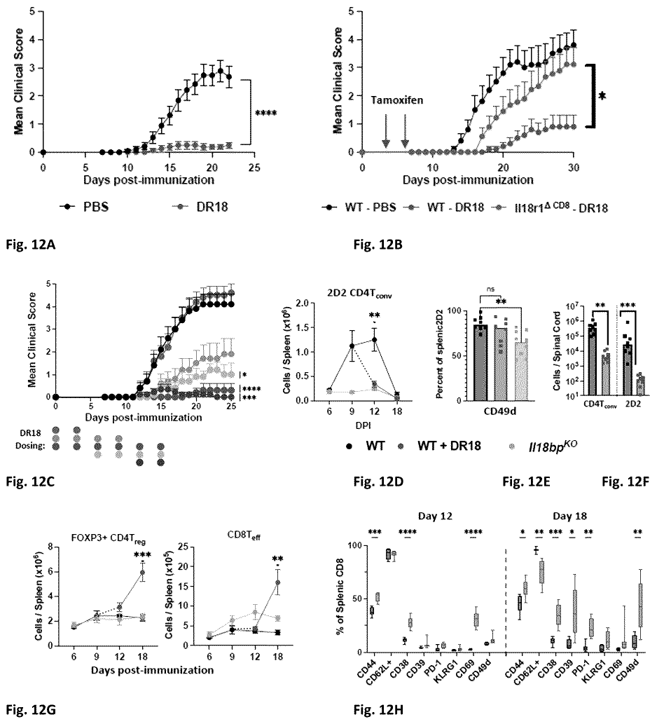

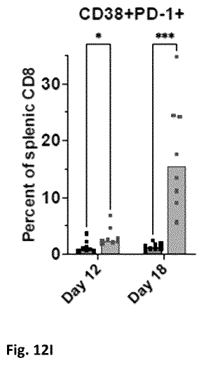

Decoy-resistant IL-18 (DR-18) is a synthetic IL-18 analogue, selected for its resistance to neutralization by IL-18BP and retained IL-18R signaling, whose human counterpart (a.k.a. ST-067) is under study as a cancer immunotherapy. DR-18 conferred near-complete protection to WT mice from paralysis and weight loss (FIG. 12A. As with Il18tg mice, protection by DR-18 was largely dependent on CD8 T-cell expression of Il18r1 (FIG. 12B). An exogenous system of protective IL-18 enabled us to interrogate granule-mediated cytotoxicity, which appears required for CD8 T-cell protection from EAE in some contexts. Perforin-deficiency did not appreciably exacerbate EAE in WT mice, nor did it significantly affect protection by DR-18 (data not shown). Therapeutic DR-18 also enabled an examination of the timing of IL-18's protection. We treated WT mice with DR-18 at various dosing schedules and found the most profound protection occurred with as little as two doses administered as late as day 12 (FIG. 12C), well after CD4Tₐᵤₜₒ induction and substantial expansion.

DR-18's ability to protect within such a short window enabled a careful, temporal examination of its effects on competing T-cell populations. Since we observed robust CD4Tₐᵤₜₒ activation and expansion in WT mice by day 12 (FIG. 7), we transferred 2D2 T-cells prior to EAE induction and then randomized WT mice to receive DR-18 or PBS. DR18 treatment from day 9 to 15 completely protected from EAE. At day 6, WT and Il18 bp^{KO} mice had similar numbers of 2D2 CD4Tₐᵤₜₒ in both spleen and peripheral blood. These 2D2 cells continued to expand in the periphery through day 12, yet they stagnated in Il18bpe^{KO} mice after day 6 (FIG. 7D and data not shown. By day 12, the number of transferred 2D2 T-cells was dramatically reduced in the spleen and PB of WT mice with only three days of DR-18 exposure. Peripheral 2D2 cells declined in all mice by day 18. Unlike our observations in 18 bp^{KO} mice, we did not see a consistent decrease in CD49d-expressing 2D2 T-cells at day 12, reinforcing the findings that IL-18 can suppress highly activated effector CD4Tₐᵤₜₒ (FIG. 12E). However, consistent with their low clinical scores, DR-18-treated and Il18 bp^{KO} mice demonstrated very few spinal cord CD4 T-cells (2D2 or endogenous) at the peak of clinical symptoms (FIG. 12F). DR-18 treatment led to the accumulation of both FOXP3+CD4Tregs and effector CD8T-cells greater than observed in control mice (FIG. 12G). The accumulation of effector CD8 T-cells was associated with profound peripheral effector CD8 T-cell activation through days 12 and 18. On day 12, CD8 T-cells from DR-18 mice demonstrate increased CD44, CD38, and CD69-expressing cells (potentially indicative of early activation). Nearly all surface markers associated with activation are increased on DR-18 treated CD8 T-cells by day 18 except for CD69.

Activation of the CD8 T-cell compartment is best seen by the substantial increase in the dual-expressing CD38⁺PD-1⁺ population previously seen in Il18 bp^{KO} mice (FIG. 12H). This population accumulated more substantially in Il18bpKO than in WT mice, and their growth was explosive following DR-18 treatment. Thus, acute DR-18 administration was dramatically protective, with an acute reciprocal shrinking of autoreactive T-cell populations and expansion of effector CD8 T-cells. Overall, these data support a model wherein excess IL-18 drives an effector response in CD8T-cells and subsequent and specific IFNγ-dependent suppression of autoreactive CD4 T-cells, preventing accumulation of CNS-toxic T-cells, histologic white matter damage, and clinical deterioration.

### DISCUSSION

“Inflammasomopathies” with chronic IL-18 elevation, such as Still's Disease, illustrate an important immunologic paradox: why doesn't chronic innate immune activation always lead to autoimmunity?Self-tolerance during innate activation is not limited to preventing autoreactive cells from developing; crucially, it involves suppressing autoreactive lymphocytes that slip through. MHC-II is consistently the strongest genetic autoimmunity risk locus, emphasizing the importance of restraining autoreactive CD4 T-cells. Foxp3⁺ CD4T_{reg} are well-recognized for this ability, but they may be most efficacious in limiting humoral autoimmunity. Opposing cellular autoimmunity, namely the direct pathogenic effects of CD4Tₐᵤₜₒ may become increasingly relevant in a future of widespread, deep B-cell depletion. Though the “T-suppressor” controversy discouraged studies of regulatory CD8 T-cells, recent discoveries have revitalized interest.

We evaluated this paradox in EAE, a model useful for preclinical MS studies and for studying in vivo competition between pathogenic CD4Tₐᵤₜₒ and multiple populations of regulatory T-cells. Though autoreactive and regulatory T-cells express the IL-18 receptor, prior EAE studies suggested a pathogenic role for IL-18 in that IL-18 deficiency and CNS delivery of IL-18BP modestly attenuated disease. Even without extra IL-18, pathogenic CD4Tₐᵤₜₒ secrete canonical IL-18-induced cytokines like IFNγ and GM-CSF, and CD4Tₐᵤₜₒ stimulated in vitro with IL-18 drive more severe EAE upon transfer.

By contrast, we found that excess IL-18 was profoundly protective across three distinct systems and two mouse colonies, opposing CD4 autoreactivity and tipping the scales against autoimmunity. We observed neither improvement nor worsening in Il18^{KO} mice and found that excess IL-18's effects on CD8 T-cells easily overcame its modest amplification of CD4Tₐᵤₜₒ (observed only in 2D2 mice). Similarly, exogenous IL-18 may also be therapeutic in the NOD model of Type 1 diabetes.

Excess IL-18's proclivity for CD8 over CD4 T-cell activation has been observed in other contexts. In models of HLH involving excess IL-18 and a variety of triggers, CD8 T-cell activation far outstripped that of CD4s. In tumor immunotherapy, DR-18 drove enhanced CD8 T-effectors and stem-like precursors to mediate tumor control. IL-18's preference for CD8's is not absolute, as observed in 2D2 mice (FIG. 2C) and in toxoplasma infection where antigen-specific CD4 T-cell responses are particularly robust. Safely harnessing protection by IL-18 will require a better understanding of the conditions required for regulatory CD8 T-cells to prevail.

Though regulatory CD8 T-cells are critical for preventing immunopathology in EAE and beyond, defining CD8T_{reg} has been complicated without a “Foxp3” of their own. We sought to characterize IL-18-activated CD8 T-cells in EAE by phenotype, antigen specificity, and effector functions. Among candidate CD8T_{reg} markers, only CD38 and Helios increased consistently with excess IL-18, and Helios was not sufficient for protection (FIG. 9E). Prior studies of protective CD38⁺CD8 T-cells identified a central-memory (Tₑₘ-like) phenotype, including expression of CD44, CD122, and CD62L. By contrast, we found CD8 T-cells exposed to excess IL-18 expressed markers consistent with CD8 T_{effector} status, like low expression of CD62L and upregulation of KLRG1, PD-1, CD39, CD49d, and CD11a. Given IL-18's potency as a driver of T_{effector} programs, it is possible that it acts on T_{cm}-like CD8T_{reg} to drive them toward an acute T_{effector} state. Alternatively, the protective CD8 T-cells may arise from a distinct population. A heterogeneous “regulatory CD8 T-cell” program is not without precedent: the CD8 T-cell exhaustion program varies substantially between tumors, chronic viral infections, or in hyperinflammation. However, varied CD8Tₑₓₕ share a core surface and transcription factor signature thus far undetected in CD8T_{reg} studies.

CD8T_{reg} may be better defined by MHC-I restriction and antigen specificity. Murine CD8T_{reg} can be restricted to either classical or non-classical MHC-I, and recognize antigens derived from autoreactive cells themselves, neuropeptides, or unidentified antigens. Notably, relapsing MS patients may have a deficit in neuropeptide-specific CD8T_{reg}. In ex vivo-stimulated splenocytes, cytokine-producing CD4 T-cells were broadly suppressed in Il18 bp^{KO} mice (FIG. 7C) whereas CD8 T-cells were only modestly affected, with slightly greater CD8 T-cell IFNg production from MOG-stimulated Il18 bp^{KO} (FIG. 10C). TCR signaling appears essential for T-cells to express the IL-18 receptor and to respond to IL-18. During LCMV infection and in Prf1-deficiency, IL-18 promoted clonality but did not favor specific clones. Thus, it remains unclear whether excess IL-18 alters the protective CD8 T-cell antigen-repertoire.

Like Foxp3⁺ CD4T_{reg}, CD8T_{reg} suppress via multiple mechanisms including contact-dependent cellular cytotoxicity (both granule- and FasL-mediated) and regulatory cytokine secretion. We observed that neither perforin nor PD-L1 signaling were necessary for IL-18 to protect. However, IFNγ neutralization abrogated protection in Il18 bp^{KO} mice, despite relatively meager increases in systemic IFNg levels (FIGS. 6D, 11I). These findings parallel observations that CD38^{HI} CD8T_{reg} require contact with target and IFNg to suppress CD4 T-cells. Overall, our data link IL-18 to the biology and significance of CD8T_{reg}, but do little to demystify their collective identity.

Consistent with EAE's stage-specific regulation by other cytokines, IL-18's protection was mediated in later phases of disease. Excess IL-18 did not impact transferred autoreactive 2D2 cells until after priming and early expansion stages (˜days 9-12). Protection did not require chronic IL-18 exposure, as acute IL-18 excess (Il18bpe^{KO} mice and DR-18 administration) was sufficient for protection, while acute loss of excess IL-18 (Il18tgIl18r1^{ΔCD8} mice) abrogated protection. Timed DR-18 administration was also instructive. Treatment around disease induction trended towards increased EAE severity, consistent with prior data suggesting IL-18's early pathogenic role. Treatment only in the late expansion/immediately presymptomatic phase was as protective as continuous dosing. Similar biphasic effects have been reported for other Type 1 cytokines, like IL-12 and IFNγ.

Geographically, excess IL-18 diminished spleen and peripheral blood CD4Tₐᵤₜₒ and reduced their expression of CD49d, a key CNS-homing integrin and MS therapeutic target. Correspondingly, spinal cord leukocyte abundance was markedly reduced across models of excess IL-18 with residual CNS T-cells resembling WT phenotypically. IL-18's effects within the CNS are complicated by IFNg's known suppressive effect on CNS cells. Together, these findings support a model where excess IL-18—via CD8 T-cells—targets highly-activated CD4Tₐᵤₜₒ in the periphery, eliminating them and/or preventing their migration into the CNS.

Inflammasomes are likely a remnant of a time when intracellular bacterial infection (e.g. Salmonella or Y. pestis) was a major selective pressure. During such infections, inflammasome products signal danger and amplify pathogen-specific T-cells, as well as any self-reactive T-cells present. IL-18's selective activation of protective CD8T-cells suggests a possible mechanism linking the decades-old observation of CD8⁺ T-suppressor cells to the purpose of opposing CD4 autoreactivity.

This study has several limitations. Though a useful autoimmune model, MOG³⁵⁻⁵⁵ induced EAE has a monophasic course and incompletely reflects rrMS. Our studies do not address excess IL-18 effects on autoreactive T-follicular/helper cells (Tfh) or B cells, relevant in other autoimmune models. Of course, our findings inspire/inform, but do not replace, the need for assessment in human cells/systems prior to consideration of clinical trials. The use of IL-18 as a cancer therapeutic could, if enough patients are exposed, identify an effect on autoimmunity (paraneoplastic or otherwise). Furthermore, while our three systems of excess IL-18—Il18tg mice, Il18 bp^{KO} mice, and DR-18—show consistent protection, we observed only partial loss of protection in (incompletely) CD8-depleted Il18bp^{KO} mice, but full loss of protection in Il18tg; Il18r1^{DCD8} mice. This likely reflects nuances of chronic vs. acute, systemic vs. local IL-18 excess that warrant further study.

Treating autoimmunity with an inflammatory cytokine like IL-18 seems counterintuitive and potentially dangerous, particularly given IL-18's association with MAS. MS is, however, the best example of successful cytokine therapy for autoimmunity: IFNβ was a mainstay of relapsing-remitting MS treatment. Notably, a small trial of systemic IFNγ in MS was not successful. We observed no evidence of hyperinflammation, and serum IFNγ levels in IL-18^{HI} mice were only modestly elevated, compared to TLR9 stimulation or acute LCMV infection. IL-18-based immunotherapies (including DR-18) are currently in clinical trials for cancer due to their ability to enhance CTL responses. In autoimmunity, we hope IL-18 will amplify endogenous CD8T_{reg} populations and help recruit new ones. Notably, the MS therapy glatiramer acetate, induces CD8T_{reg} as part of its mechanism, supporting the therapeutic relevance of this pathway. Rather than systemic administration, IL-18 may be helpful in preparing cellular therapies, aligning with other immunotherapeutic strategies to boost regulatory cells. In humans, low-dose IL-2 expanded CD4T_{reg}, whereas IL-15 expanded CD8T_{reg} and protected against autoimmunity in preclinical models. Thus, IL-18 may be a crucial part of the ex vivo preparation of protective, therapeutic CD8 T-cells.

Comparative study of IEI inspired us to investigate why the chronic innate immune activation in some autoinflammatory disorders, namely those with chronic inflammasome-mediated overproduction of IL-18, seems to defy promoting autoimmunity. We found that, in EAE, excess IL-18 actively opposes autoreactive cells and averts autoimmune damage. It does so specifically via CD8 T-cells and IFNγ, and after the onset and expansion of CD4 T-cell autoreactivity. Though grounded in human observations, these findings require further study to determine the generalizability, mechanisms, and practicality of IL-18 agonism as a treatment strategy in autoimmune diseases—particularly those directly mediated by CD4Tₐᵤₜₒ and opposed by protective CD8 T-cells. Nevertheless, just as it is proving a potent adjunct to T-cell therapy for cancer, IL-18's preferential activation of CD8 T-cells provides a useful tool in recruiting the protective functions of CD8 T-cells to the fight against autoimmunity.

### REFERENCES

1. Weiss, E. S. et al. Interleukin-18 diagnostically distinguishes and pathogenically promotes human and murine macrophage activation syndrome. Blood 131, 1442-1455, doi:10.1182/blood-2017-12-820852 (2018).
2. Xia, Y. et al. Extremely elevated IL-18 levels may help distinguish systemic-onset juvenile idiopathic arthritis from other febrile diseases. Braz J Med Biol Res 50, e5958, doi:10.1590/1414-431X20165958 (2017).
3. Girard, C. et al. Elevated serum levels of free interleukin-18 in adult-onset Still's disease. Rheumatology (Oxford) 55, 2237-2247, doi:10.1093/rheumatology/kew300 (2016).
4. Canna, S. W. et al. An activating NLRC4 inflammasome mutation causes autoinflammation with recurrent macrophage activation syndrome. Nat Genet 46, 1140-1146, doi:10.1038/ng.3089 (2014).
5. Stone, D. L. et al. Excess Serum Interleukin-18 Distinguishes Patients With Pathogenic Mutations in PSTPIP1. Arthritis & rheumatology 74, 353-357, doi:10.1002/art.41976 (2022).
6. Wada, T. et al. Sustained elevation of serum interleukin-18 and its association with hemophagocytic lymphohistiocytosis in XIAP deficiency. Cytokine 65, 74-78, doi:10.1016/j.cyto.2013.09.007 (2014).
7. Gernez, Y. et al. Severe autoinflammation in 4 patients with C-terminal variants in cell division control protein 42 homolog (CDC42) successfully treated with IL-1beta inhibition. The Journal of allergy and clinical immunology 144, 1122-1125 e1126, doi:10.1016/j.jaci.2019.06.017 (2019).
8. Lam, M. T. et al. A novel disorder involving dyshematopoiesis, inflammation, and HLH due to aberrant CDC42 function. J Exp Med 216, 2778-2799, doi:10.1084/jem.20190147 (2019).
9. Dendrou, CA, Petersen, J, Rossjohn, J, Fugger, L. Hla variation and disease. Nat Rev Immun; 2018; 18: 325-339].
10. Onodera A, Kokubo K, Okano M, Onoue M, Kiuchi M, Iwamura C, et al. Pathogenic helper T cells as the novel therapeutic targets for immune-mediated intractable diseases. Pharmacol Ther. 2023; 247:108445.
11. Nakagawa H, Wang L, Cantor H, Kim H J. New Insights Into the Biology of CD8 Regulatory T Cells. Adv Immunol. 2018; 140:1-20.
12. Saligrama N, Zhao F, Sikora M J, Serratelli W S, Fernandes R A, Louis D M, et al. Opposing T cell responses in experimental autoimmune encephalomyelitis. Nature. 2019; 572(7770):481-7.
13. Li J, Zaslavsky M, Su Y, Guo J, Sikora M J, van Unen V, et al. KIR(+)CD8(+) T cells suppress pathogenic T cells and are active in autoimmune diseases and COVID-19. Science. 2022; 376(6590):eabi9591.
14. Zhou T, Damsky W, Weizman O E, McGeary M K, Hartmann K P, Rosen C E, et al. IL-18BP is a secreted immune checkpoint and barrier to IL-18 immunotherapy. Nature. 2020; 583(7817):609-14.
15. Tsoukas P, Rapp E, Van Der Kraak L, Weiss E S, Dang V, Schneider C, et al. Interleukin-18 and cytotoxic impairment are independent and synergistic causes of murine virus-induced hyperinflammation. Blood. 2020; 136(19):2162-74.
16. Landy E, Varghese J, Dang V, Szymczak-Workman A, Kane L P, Canna S W. Complementary HLH susceptibility factors converge on CD8 T-cell hyperactivation. Blood Adv. 2023; 7(22):6949-63.
17. Shimizu M, Nakagishi Y, Inoue N, Mizuta M, Ko G, Saikawa Y, et al. Interleukin-18 for predicting the development of macrophage activation syndrome in systemic juvenile idiopathic arthritis. Clin Immunol. 2015; 160(2):277-81.
18. Hoshino T, Kawase Y, Okamoto M, Yokota K, Yoshino K, Yamamura K, et al. Cutting edge: IL-18-transgenic mice: in vivo evidence of a broad role for IL-18 in modulating immune function. J Immunol. 2001; 166(12):7014-8.
19. Andrews L P, Vignali K M, Szymczak-Workman A L, Burton A R, Brunazzi E A, Ngiow S F, et al. A Cre-driven allele-conditioning line to interrogate CD4(+) conventional T cells. Immunity. 2021; 54(10):2209-17 e6.
20. Hu B, Ren J, Luo Y, Keith B, Young R M, Scholler J, et al. Augmentation of Antitumor Immunity by Human and Mouse CAR T Cells Secreting IL-18. Cell reports. 2017; 20(13):3025-33.
21. Girard-Guyonvarc'h C, Palomo J, Martin P, Rodriguez E, Troccaz S, Palmer G, et al. Unopposed IL-18 signaling leads to severe TLR9-induced macrophage activation syndrome in mice. Blood. 2018; 131(13):1430-41.
22. Novick D, Elbirt D, Miller G, Dinarello C A, Rubinstein M, Sthoeger Z M. High circulating levels of free interleukin-18 in patients with active SLE in the presence of elevated levels of interleukin-18 binding protein. J Autoimmun. 2010; 34(2):121-6.
23. Novick D, Kim S, Kaplanski G, Dinarello C A. Interleukin-18, more than a Th1 cytokine. Seminars in immunology. 2013; 25(6):439-48.
24. Gracie J A, Forsey R J, Chan W L, Gilmour A, Leung B P, Greer M R, et al. A proinflammatory role for IL-18 in rheumatoid arthritis. J Clin Invest. 1999; 104(10):1393-401.
25. Ortega S B, Kashi V P, Tyler A F, Cunnusamy K, Mendoza J P, Karandikar N J. The disease-ameliorating function of autoregulatory CD8 T cells is mediated by targeting of encephalitogenic CD4 T cells in experimental autoimmune encephalomyelitis. J Immunol. 2013; 191(1):117-26.
26. Wang J, Sun C, Gerdes N, Liu C, Liao M, Liu J, et al. Interleukin 18 function in atherosclerosis is mediated by the interleukin 18 receptor and the Na—Cl co-transporter. Nat Med. 2015; 21(7):820-6.
27. Mertens R T, Misra A, Xiao P, Baek S, Rone J M, Mangani D, et al. A metabolic switch orchestrated by IL-18 and the cyclic dinucleotide cGAMP programs intestinal tolerance. Immunity. 2024.
28. Zhang X, Luo S, Wang M, Cao Q, Zhang Z, Huang Q, et al. Differential IL18 signaling via IL18 receptor and Na—Cl co-transporter discriminating thermogenesis and glucose metabolism regulation. Nat Commun. 2022; 13(1):7582.
29. Clark J T, Weizman O E, Aldridge D L, Shallberg L A, Eberhard J, Lanzar Z, et al. IL-18BP mediates the balance between protective and pathological immune responses to Toxoplasma gondii. Cell reports. 2023; 42(3):112147.
30. Lipinski B, Unmuth L, Arras P, Becker S, Bauer C, Toleikis L, et al. Generation and engineering of potent single domain antibody-based bispecific IL-18 mimetics resistant to IL-18BP decoy receptor inhibition. MAbs. 2023; 15(1):2236265.
31. Poholek C H, Raphael I, Wu D, Revu S, Rittenhouse N, Uche U U, et al. Noncanonical STAT3 activity sustains pathogenic Th17 proliferation and cytokine response to antigen. J Exp Med. 2020; 217(10).
32. Kinoshita M, Kuranaga N, Matsumoto A, Ono S, Shinomiya N, Hiraide H, et al. Multiple interleukin-18 injections promote both mouse Th1 and Th2 responses after sublethal Escherichia coli infection. Clin Exp Immunol. 2006; 143(1):41-9.
33. Manglani M, Gossa S, McGavern D B. Leukocyte Isolation from Brain, Spinal Cord, and Meninges for Flow Cytometric Analysis. Curr Protoc Immunol. 2018; 121(1):e44.
34. Richner M, Jager S B, Siupka P, Vaegter C B. Hydraulic Extrusion of the Spinal Cord and Isolation of Dorsal Root Ganglia in Rodents. J Vis Exp. 2017(119).
35. Kim H J, Barnitz R A, Kreslavsky T, Brown F D, Moffett H, Lemieux M E, et al. Stable inhibitory activity of regulatory T cells requires the transcription factor Helios. Science. 2015; 350(6258):334-9.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for ameliorating disease activity in autoimmune, allergic, or post-transplant disorders in a subject in need thereof, comprising; administering to said subject an effective amount of an agent that increases the immune protective effects of Interleukin-18 (IL-18) to promote one or more of differentiation, expansion, and function of therapeutically beneficial IL-18 responsive lymphocytes, thereby suppressing immunopathology caused by pathogenic lymphocytes in said subject, and improving inflammatory symptoms.

2. A method for treating an autoimmune disease caused by a harmful pro-inflammatory response to a self-antigen in a subject in need thereof, comprising; administering to a subject having the self-antigen, an interleukin-18 receptor (IL-18R) agonist in an amount effective to ameliorate a symptom of the autoimmune disease, thereby improving autoimmune symptoms mediated in said subject.

3. A method for treating an inflammatory disease in a subject, comprising administering to said subject an effective amount of an agent that enhances the immune protective effects of Interleukin-18 (IL-18) indirectly via effects on antigen presenting cell function, thereby improving an inflammatory disease mediated by an indirectly-acting APC signal.

4. A method for treating an inflammatory disease caused by pathogenic CD4+ T cells in a subject in need thereof, comprising
administering to said subject an effective amount of a therapeutic population of cells comprising one or more of CD8+ T-cells, Natural Killer (NK) cells, CD4+ regulatory T-cells, cultured or conditioned in the presence of an agonist that mimics or enhances the immune protective effects of IL-18; optionally the method further comprising contacting said one or more of CD8+ T-cells, Natural Killer (NK) cells, CD4+ regulatory T-cells with said agonist; thereby suppressing immunopathology resulting from the pathogenic CD4+ T cells present in said subject.

5. A method for treating an inflammatory disease caused by pathogenic CD4+ T cells in a subject in need thereof, comprising administering to said subject an effective amount of an antagonist that inhibits activity of IL-18 Binding Protein (IL-18BP), thereby increasing at least one of bioactivity and enhancement of immune protective effects of free IL-18 in said subject, thereby suppressing immunopathology resulting from the pathogenic CD4+ T cells and alleviating inflammatory symptoms in said subject.

6. The method of any one of claims 1-5, wherein said one or more lymphocyte populations is selected from a suppressive CD8+ T cell, Natural Killer (NK) cell, NKT cell, and CD4+ regulatory T-cells.

7. The method of any of one of claims 1-6, wherein the disease or disorder is selected from the group consisting of: multiple sclerosis, rheumatoid arthritis, myasthenia gravis, Addison's disease, celiac disease, type 1 diabetes mellitus, autoimmune thyroiditis, ulcerative colitis, Sjogren syndrome, systemic lupus erythematosus, Grave's disease, Crohn's disease, Waldenstrom's macroglobulinemia, hyperviscosity syndrome, monoclonal gammopathy of undetermined origin, POEMS syndrome, myeloma, and macroglobulinemia.

8. The method of claim 1, or claim 3, or claim 5, wherein said CD4+ cells are autoreactive.

9. The method of any one of claim 1 to claim 6, wherein said allergic disorder is selected from atopic dermatitis, allergic rhinosinusitis, allergic asthma, and eosinophilic esophagitis

10. The method of any one of claims 1 to 6, wherein said post-transplant disorder is graft versus host disease (GvHD).

11. The method of any one of claims 1 to 10, wherein bioactivity of free IL-18 is increased in said subject.

12. The method of any one of claims 1 to 11, further comprising administration of at least one additional agent, wherein the at least one additional agent comprises one or more of IL-18, IL-18 receptor agonists and IL-18BP antagonist.

13. The method of claim 11 or 12, wherein bioactivity of free IL-18 is increased and IL-18BP binding is reduced.

14. The method of any one of claims 1 to 13, wherein an IL-18 receptor agonists that is an IL-18 mimetic is administered.

15. The method of any one of claims 1 to 14, wherein said agent, said IL-18R agonist, or said agonist is decoy resistant IL-18 (DR-18).

16. The method of claim 15, wherein the DR-18 comprises an amino acid sequence having at least 85%, at least 90%, at least 91%, at least 92%, at least 93%, at least 94%, at least 95%, at least 96%, at least 97%, at least 98%, at least 99%, or 100% sequence identity with at least one of SEQ ID NOs: 2-63.

17. The method of claim 16, wherein the DR-18 comprises one or more mutations at positions Y1, L5, K8, C38, M51, K53, S55, Q56, P57, G59, M60, C68, E77, Q103, S105, D110, N111, M113, V153, and N155 relative to the wild-type IL-18 amino acid sequence set forth in SEQ ID NO:108.

18. The method of claim 17, wherein the DR-18 comprises an amino acid sequence selected from SEQ ID NOs: 20, 7, 17-19, 21, 22, 55-57, 62 and 63.

19. The method of any one of claims 15 to 18, wherein the amount of the DR-18 administered is from 15 μg/kg to 3000 μg/kg.

20. The method of any one of the previous claims, wherein said agent, agonist or antagonist is administered via a route selected from systemic, intravenous, or subcutaneous administration.

21. The method of claim 6, wherein the differentiation, expansion, and, or function of suppressive CD8+ T cells induced by said agent is monitored.

22. The method of claim 3, wherein signaling from said APC engages MHC class II molecules which interact with T cell receptors, said APC being selected from dendritic cells, macrophages, B cells, thymic epithelial cells, vascular endothelial cells, and follicular dendritic cells.

23. The method of any of the previous claims, wherein said immune protective effects of free IL-18 include one or more of
i) binding of IL-18 to the IL-18R and activation of the IL-18R;
ii) regulation of innate and acquired immune responses;
iii) induction of one or more T-lymphocyte helper (Th1) responses;
iv) enhanced cell-mediated cytotoxicity;
v) IFN-γ induction;
vi) enhanced production of GM-CSF and IL-2;
vii) potentiation of anti-CD3 induced T-cell proliferation;
viii) increased Fas-mediated killing by natural killer cells (NK cells) and CD4+ Th1 cells;
ix) increased apoptotic death via activation of the Fas-FasL pathway signaling;
x) up-regulation of FasL expression;
xi) induction of T-lymphocyte helper cell type 2 responses (Th2) in T-cells and NK cells;
xii) stimulation of basophils and mast cells to produce Th2 cytokines and histamine;
xiii) inhibition of IgE production; and
xiv) activation of T cell signaling pathways by APC cells.

24. The method of claim 5, wherein said antagonist is selected from an inhibitory nucleic acid, an inhibitory antibody, a peptide mimetic, a small molecule, or an IL-18 decoy-to-the-decoy (D2D).

25. The method of claim 24, wherein the IL-18 D2D comprises an amino acid sequence having at least 85%, at least 90%, at least 91%, at least 92%, at least 93%, at least 94%, at least 95%, at least 96%, at least 97%, at least 98%, or at least 99% sequence identity with at least one of SEQ ID NOs: 64-97.

26. The method of claim 25, wherein the DR-18 comprises an amino acid sequence selected from SEQ ID NOs: 64-97.

27. The method of any one of the previous claims, further comprising administration of one or more anti-inflammatory agents selected from analgesic agents, disease-modifying anti-rheumatic drugs (DMARDs), non-steroidal anti-inflammatory drugs (NSAIDs).

28. The method of claim 27, further comprising administration of one or more antiviral, antibiotic, analgesic, corticosteroid, antagonists of inflammatory cytokine, a non-steroidal anti-inflammatory agent, pentoxifylline, thalidomide, azathioprine, cyclophosphamide, cyclosporine, hydroxychloroquine sulfate, methotrexate, leflunomide, minocycline, penicillamine, sulfasalazine, indomethacin; celecoxib; rituxin, rofecoxib; ketorolac; nambumetone; piroxicam; naproxen; oxaprozin; sulindac; ketoprofen; diclofenac; and gold compounds such as oral gold, gold sodium thiomalate, and aurothioglucose.

29. The method of claim 4, wherein said culturing or said conditions produce Suppressive CD122+CD8+ T-cells and/or KIR+CD8+ T-cells.

30. The method of any one of the previous claims, wherein said cells are donor cells haplotype-matched to the subject to be treated.

31. The method of any one of the previous claims wherein said cells are autologous and harvested from the subject to be treated.

32. Use of a composition comprising an agonist of interleukin 18 (IL-18) or IL-18 receptor (IL-18R), in a method of treating a subject, according to any one of the preceding claims, wherein the subject is a human subject having a disease or disorder selected from the group consisting of: autoimmune disease or disorder, allergic disease or disorder, post-transplant disease or disorder, and an inflammatory disease caused by pathogenic CD4+ T cells; and the method treats the subject for the disease or disorder.

33. The use according to claim 32, wherein the disease or disorder is multiple sclerosis.

34. The use according to claim 32 or 33, wherein the agonist is a decoy resistant IL-18 (DR-18).

35. The use according to claim 34, wherein the DR-18 comprises an amino acid sequence selected from SEQ ID NOs: 20, 7, 17-19, 21, 22, 55-57, 62 and 63.

36. Use of a composition comprising an antagonist of interleukin 18 binding protein (IL-18BP), in a method of treating a subject, according to any one of the preceding claims, wherein the subject is a human subject having a disease or disorder selected from the group consisting of: autoimmune disease or disorder, allergic disease or disorder, post-transplant disease or disorder, and an inflammatory disease caused by pathogenic CD4+ T cells; and the method treats the subject for the disease or disorder.

37. The use according to claim 36, wherein the disease or disorder is multiple sclerosis.

38. The use according to claim 36 or 37, wherein the antagonist is an interleukin 18 decoy-to-the-decoy (IL-18 D2D).

39. The use according to claim 36 or 37, wherein the antagonist is an IL-18BP inhibitory antibody that specifically binds IL-18BP.

40. The use according to claim 36 or 37, wherein the antagonist is an IL-18BP inhibitory nucleic acid that prevents translation and/or expression of a nucleic acid encoding IL-18BP.


SEQUENCE LISTING (108 sequences)

SEQ ID NO: 1 - protein, 194 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 2 - protein, 157 aa, synthetic construct
YFGKLESKLSVIRNLNDQVLFIDQGNRPLFEDMTDSDSRDNAPRTIFIISKYSDSLARGLAVTISVKSEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 3 - protein, 157 aa, synthetic construct
YFGKLESKLSVIRNLNDQVLFIDQGNRPLFEDMTDSDCRDNAPRTIFIISKYSDSLARGLAVTISVKSEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 4 - protein, 157 aa, synthetic construct
YFGKLESKLSVIRNLNDQVLFIDQGNRPLFEDMTDSDSRDNAPRTIFIISKYSDSLARGLAVTISVKCEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 5 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 6 - protein, 157 aa, synthetic construct
YFGKLESKLSVIRNLNDQVLFIDQGNRPLFEDMTDSDSRDNAPRTIFIISKYSDSLARGLAVTISVKSEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 7 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 8 - protein, 157 aa, synthetic construct
YFGKLESKLSVIRNLNDQVLFIDQGNRPLFEDMTDSDSRDNAPRTIFIISKYSDSLARGLAVTISVKCEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 9 - protein, 157 aa, synthetic construct
YFGKLESKLSVIRNLNDQVLFIDQGNRPLFEDMTDSDSRDNAPRTIFIISKYSDSLARGLAVTISVKCEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 10 - protein, 157 aa, synthetic construct
YFGKLESKLSVIRNLNDQVLFIDQGNRPLFEDMTDSDCRDNAPRTIFIISKYSDSLARGLAVTISVKSEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 11 - protein, 157 aa, synthetic construct
YFGKLESKLSVIRNLNDQVLFIDQGNRPLFEDMTDSDCRDNAPRTIFIISKYSDSLARGLAVTISVKSEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 12 - protein, 157 aa, synthetic construct
YFGKLESKLSVIRNLNDQVLFIDQGNRPLFEDMTDSDCRDNAPRTIFIISKYSDSLARGLAVTISVKCEKISTLSSENKIISFKEMNPPDNIKDTKSDIIFFQRDVPGHSRKMQFESSSYEGYFLASEKERDLFKLILKKEDELGDRSIMFTVQNED

SEQ ID NO: 13 - protein, 157 aa, synthetic construct
YFGKLESKLSVIRNLNDQVLFIDQGNRPLFEDMTDSDSRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 14 - protein, 157 aa, synthetic construct
YFGKLESKLSVIRNLNDQVLFIDQGNRPLFEDMTDSDCRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 15 - protein, 157 aa, synthetic construct
YFGKLESKLSVIRNLNDQVLFIDQGNRPLFEDMTDSDCRDNAPRTIFIISKYSDSLARGLAVTISVKCEKISTLSSENKIISFKEMNPPDNIKDTKSDIIFFQRDVPGHSRKMQFESSSYEGYFLACEKERDLFKLILKKEDELGDRSIMFTVQNED

SEQ ID NO: 16 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLASEKERDLFKLILKKEDELGDRSIMFTVQNED

SEQ ID NO: 17 - protein, 157 aa, synthetic construct
YFGKLESKLSVIRNLNDQVLFIDQGNRPLFEDMTDSDSRDNAPRTIFIISKYSDSLARGLAVTISVKGEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 18 - protein, 157 aa, synthetic construct
YFGKLESKLSVIRNLNDQVLFIDQGNRPLFEDMTDSDSRDNAPRTIFIISKYSDSLARGLAVTISVKAEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 19 - protein, 157 aa, synthetic construct
YFGKLESKLSVIRNLNDQVLFIDQGNRPLFEDMTDSDSRDNAPRTIFIISKYSDSLARGLAVTISVKVEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 20 - protein, 157 aa, synthetic construct
YFGKLESKLSVIRNLNDQVLFIDQGNRPLFEDMTDSDSRDNAPRTIFIISKYSDSLARGLAVTISVKDEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 21 - protein, 157 aa, synthetic construct
YFGKLESKLSVIRNLNDQVLFIDQGNRPLFEDMTDSDSRDNAPRTIFIISKYSDSLARGLAVTISVKEEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 22 - protein, 157 aa, synthetic construct
YFGKLESKLSVIRNLNDQVLFIDQGNRPLFEDMTDSDSRDNAPRTIFIISKYSDSLARGLAVTISVKNEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 23 - protein, 157 aa, synthetic construct
YFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 24 - protein, 157 aa, synthetic construct
YFGKLESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 25 - protein, 157 aa, synthetic construct
RFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 26 - protein, 157 aa, synthetic construct
RFGKLESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRNVPGHKYKMQFESSSYEGYFLACEKERDLFKLILKKEDELGDRSIMFTVQNED

SEQ ID NO: 27 - protein, 157 aa, synthetic construct
YFGKLESQLSVIRNLNDQVLFIDQGNRPLFEDMTDSDCRDNAPRTIFIISTYKDKQPRTKAVTISVKCEKISTLSCENKIISFKEMNPPDNIKDTKSDIIFFQRRVPGHHNKMQFESSSYEGYFLACEKERDLFKLILKKEDELGDRSIMFTVQKED

SEQ ID NO: 28 - protein, 157 aa, synthetic construct
YFGKLESRLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 29 - protein, 157 aa, synthetic construct
YFGKLESRLSVIRNLNDQVLFIDQGNRPLFEDMTDSDCRDNAPRTIFIISDYKDKQPRAXAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 30 - protein, 157 aa, synthetic construct
YFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHNNKMQFESSSYEGYFLACEKERDLFKLILKKEDELGDRSIMFTTQNED

SEQ ID NO: 31 - protein, 157 aa, synthetic construct
YFGKIESKLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRKVPGHQHKMQFESSSYEGYFLACEKERDLFKLILKKEDELGDRSIMFTVQKED

SEQ ID NO: 32 - protein, 157 aa, synthetic construct
YFGKIESKLSVIRNLNDQVLFIDQGNRPLFEDMTDSDCRDNAPRTIFIISTYKDRQPRGKAVTISVKCEKISTLSCENKIISFKEMNPPDNIKDTKSDIIFFERDVPGHHHKMQFESSSYEGYFLACEKERDLFKLILKKEDELGDRSIMFTIQNED

SEQ ID NO: 33 - protein, 157 aa, synthetic construct
YFGKIESKLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHKHKMQFESSSYEGYFLACEKERDLFKLILKKEDELGDRSIMFTTQHED

SEQ ID NO: 34 - protein, 157 aa, synthetic construct
YFGKIESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRRVPGHHHKMQFESSSYEGYFLACEKERDLFKLILKKEDELGDRSIMFTIQKED

SEQ ID NO: 35 - protein, 157 aa, synthetic construct
YFGKIESRLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHDYKMQFESSSYEGYFLACEKERDLFKLILKKEDELGDRSIMFTIQKED

SEQ ID NO: 36 - protein, 157 aa, synthetic construct
YFGKYESRLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHEHKMQFESSSYEGYFLACEKERDLFKLILKKEDELGDRSIMFTVQKED

SEQ ID NO: 37 - protein, 157 aa, synthetic construct
HFGKYESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHHNKMQFESSSYEGYFLACEKERDLFKLILKKEDELGDRSIMFTVQKED

SEQ ID NO: 38 - protein, 157 aa, synthetic construct
RFGKLESKLSVIRNLNDQVLFIDQGNRPLFEDMTDSDCRDNAPRTIFIISTYRDSQPRAKAVTISVKCEKISTLSCENKIISFKEMNPPDNIKDTKSDIIFFQRDVPGHQHKMQFESSSYEGYFLACEKERDLFKLILKKEDELGDRSIMFTAQKED

SEQ ID NO: 39 - protein, 157 aa, synthetic construct
RFGKLESRLSVIRNLNDQVLFIDQGNRPLFEDMTDSDCRDNAPRTIFIISDYRDSQPRGRAVTISVKCEKISTLSCENKIISFKEMNPPDNIKDTKSDIIFFKRNVPGHKYKMQFESSSYEGYFLACEKERDLFKLILKKEDELGDRSIMFTVQHED

SEQ ID NO: 40 - protein, 157 aa, synthetic construct
RFGKLESRLSVIRNLNDQVLFIDQGNRPLFEDMTDSDCRDNAPRTIFIISNYRDSQPRGQAVTISVKCEKISTLSCENKIISFKEMNPPDNIKDTKSDIIFFKRRVPGHNHKMQFESSSYEGYFLACEKERDLFKLILKKEDELGDRSIMFTVQKED

SEQ ID NO: 41 - protein, 157 aa, synthetic construct
RFGKLESRLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 42 - protein, 157 aa, synthetic construct
RFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFERNVPGHKYKMQFESSSYEGYFLACEKERDLFKLILKKEDELGDRSIMFTVQNED

SEQ ID NO: 43 - protein, 157 aa, synthetic construct
RFGKLESRLSVIRNLNDQVLFIDQGNRPLFEDMTDSDCRDNAPRTIFIISTYRDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQXED

SEQ ID NO: 44 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRNVPGHHDKMQFESSSYEGYFLACEKERDLFKLILKKEDELGDRSIMFTVQHED

SEQ ID NO: 45 - protein, 157 aa, synthetic construct
RFGKLESRLSVIRNLNDQVLFIDQGNRPLFEDMTDSDCRDNAPRTIFIISTYKDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQKED

SEQ ID NO: 46 - protein, 157 aa, synthetic construct
RFGKYESKLSVIRNLNDQVLFIDQGNRPLFEDMTDSDCRDNAPRTIFIISTYKDSQPRTKAVTISVKCEKISTLSCDNKIISFKEMNPPDNIKDTKSDIIFFQRDVPGHKHKMQFESSSYEGYFLACEKERDLFKLILKKEDELGDRSIMFTVQNED

SEQ ID NO: 47 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRKVPGHNHKMQFESSSYEGYFLACEKERDLFKLILKKEDELGDRSIMFTVQKED

SEQ ID NO: 48 - protein, 157 aa, synthetic construct
YFGKLESKLSVIRNLNDQVLFIDQGNRPLFEDMTDSDCRDNAPRTIFIISEYKDSELRGRAVTISVKCEKISTLSCENKIISFKEMNPPDNIKDTKSDIIFFPRAVPGHNRKVQFESSSYEGYFLACEKERDLFKLILKKEDELGDRSIMFTVQNED

SEQ ID NO: 49 - protein, 157 aa, synthetic construct
YFGKLESKLSVIRNLNDQVLFIDQGNRPLFEDMTDSDCRDNAPRTIFIISKYKDSAGRGLAVTISVKCEKISTLSCENKIISFKEMNPPDNIKDTKSDIIFFERDVPGHSNKVQFESSSYEGYFLACEKERDLFKLILKKEDELGDRSIMFTVQNED

SEQ ID NO: 50 - protein, 157 aa, synthetic construct
YFGKLESKLSVIRNLNDQVLFIDQGNRPLFEDMTDSDCRDNAPRTIFIISKYGDSAARGLAVTISVKCEKISTLSCENKIISFKEMNPPDNIKDTKSDIIFFQRSVPGHKRKMQFESSSYEGYFLACEKERDLFKLILKKEDELGDRSIMFTVQNED

SEQ ID NO: 51 - protein, 157 aa, synthetic construct
YFGKLESKLSVIRNLNDQVLFIDQGNRPLFEDMTDSDCRDNAPRTIFIISKYGDSRGRGLAVTISVKCEKISTLSCENKIISFKEMNPPDNIKDTKSDIIFFERDVPGHNSKRQFESSSYEGYFLACEKERDLFKLILKKEDELGDRSIMFTVQNED

SEQ ID NO: 52 - protein, 157 aa, synthetic construct
YFGKLESKLSVIRNLNDQVLFIDQGNRPLFEDMTDSDCRDNAPRTIFIISKYGDSVPRGLAVTISVKCEKISTLSCENKIISFKEMNPPDNIKDTKSDIIFFARAVPGHSRKTQFESSSYEGYFLACEKERDLFKLILKKEDELGDRSIMFTVQNED

SEQ ID NO: 53 - protein, 157 aa, synthetic construct
YFGKLESKLSVIRNLNDQVLFIDQGNRPLFEDMTDSDCRDNAPRTIFIISKYSDSGARGLAVTISVKCEKISTLSCENKIISFKEMNPPDNIKDTKSDIIFFARAVPGHGRKTQFESSSYEGYFLACEKERDLFKLILKKEDELGDRSIMFTVQNED

SEQ ID NO: 54 - protein, 157 aa, synthetic construct
YFGKLESKLSVIRNLNDQVLFIDQGNRPLFEDMTDSDCRDNAPRTIFIISKYSDSKARGMAVTISVKCEKISTLSCENKIISFKEMNPPDNIKDTKSDIIFFARDVPGHSSKRQFESSSYEGYFLACEKERDLFKLILKKEDELGDRSIMFTVQNED

SEQ ID NO: 55 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 56 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFQRNVPGHGRKMQFESSSYEGYFLACEKERDLFKLILKKEDELGDRSIMFTVQNED

SEQ ID NO: 57 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARSVPGHGRKTQFESSSYEGYFLACEKERDLFKLILKKEDELGDRSIMFTVQNED

SEQ ID NO: 58 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARDVPGHSGKRQFESSSYEGYFLACEKERDLFKLILKKEDELGDRSIMFTVQNED

SEQ ID NO: 59 - protein, 157 aa, synthetic construct
YFGKLESKLSVIRNLNDQVLFIDQGNRPLFEDMTDSDCRDNAPRTIFIISKYTDSRPRGLAVTISVKCEKISTLSCENKIISFKEMNPPDNIKDTKSDIIFFERDVPGHSSKKQFESSSYEGYFLACEKERDLFKLILKKEDELGDRSIMFTVQNED

SEQ ID NO: 60 - protein, 157 aa, synthetic construct
YFGKLESKLSVIRNLNDQVLFIDQGNRPLFEDMTDSDCRDNAPRTIFIISKYTDSRARGMAVTISVKCEKISTLSCENKIISFKEMNPPDNIKDTKSDIIFFERDVPGHNDKRQFESSSYEGYFLACEKERDLFKLILKKEDELGDRSIMFTVQNED

SEQ ID NO: 61 - protein, 157 aa, synthetic construct
YFGKLESKLSVIRNLNDQVLFIDQGNRPLFEDMTDSDCRDNAPRTIFIISRYKDSGKRGLAVTISVKCEKISTLSCENKIISFKEMNPPDNIKDTKSDIIFFRRSVPGHSRKVQFESSSYEGYFLACEKERDLFKLILKKEDELGDRSIMFTVQNED

SEQ ID NO: 62 - protein, 157 aa, synthetic construct
YFGKLESKLSVIRNLNDQVLFIDQGNRPLFEDMTDSDCRDNAPRTIFIISKYGDSGARGLAVTISVKCEKISTLSCENKIISFKEMNPPDNIKDTKSDIIFFERDVPGHSGKVQFESSSYEGYFLACEKERDLFKLILKKEDELGDRSIMFTVQNED

SEQ ID NO: 63 - protein, 157 aa, synthetic construct
YFGKLESKLSVIRNLNDQVLFIDQGNRPLFEDMTDSDCRDNAPRTIFIISKYGDSRPRGMAVTISVKCEKISTLSCENKIISFKEMNPPDNIKDTKSDIIFFQRAVPGHNRKMQFESSSYEGYFLACEKERDLFKLILKKEDELGDRSIMFTVQNED

SEQ ID NO: 64 - protein, 157 aa, synthetic construct
HFGKLESKLSVIRNLNGQVLFIDQGNRPLFKDMTASDCRANAPRTIFIISFYKDSQPRGMAVTISVKCEKISTLSCENKIISFKEMNPPDNIKDTKSDIIFFIRSVPGADNKFQFESSSYEGYFLACEKERDLFKLILKKEDELGDRSIMFTVQNED

SEQ ID NO: 65 - protein, 157 aa, synthetic construct
DFGKLESKLSVIRNLNDQVLFIDQGNRPLFADMTDNPCRSNAPRTIFIISFYKDSQPRGIAVTISVKCEKISTLSCENKIISFKEMNPPDNIKDTKSDIIFFLRSVPGPDNKMQFESSSYEGYFLACEKERDLFKLILKKEDELGDRSIMFTVQNED

SEQ ID NO: 66 - protein, 157 aa, synthetic construct
HFGKLESKLSVIRNLNGQVLFIDQGNRPLFADMEASPCRDNAPRTIFIISFYKDSQPRGLAVTISVKCEKISTLSCENKIISFKEMNPPDNIKDTKSDIIFFLRSVPGHDNKMQFESSSYEGYFLACEKERDLFKLILKKEDELGDRSIMFTVQNED

SEQ ID NO: 67 - protein, 157 aa, synthetic construct
LFGKLESKLSVIRNLNGQVLFIDQGNRPLFADMTSSPCRSRAPRTIFIISFYKDSQPRGFAVTISVKCEKISTLSCENKIISFKEMNPPDNIKDTKSDIIFFIRSVPGHDNKIQFESSSYEGYFLACEKERDLFKLILKKEDELGDRSIMFTVQNED

SEQ ID NO: 68 - protein, 157 aa, synthetic construct
HFGKLESKLSVIRNLNDQVLFIDQGNRPLFTDMESKPCRDSAPRTIFIISMYKDSQPRGIAVTISVKCEKISTLSCENKIISFKEMNPPDNIKDTKSDIIFFIRSVPGHDNKFQFESSSYEGYFLACEKERDLFKLILKKEDELGDRSIMFTVQNED

SEQ ID NO: 69 - protein, 157 aa, synthetic construct
YFGKLESKLSVIRNLNRQVLFIDQGNRPLFTDMTYKDCRDNAPRTIFIISFYKDSQPRGFAVTISVKCEKISTLSCENKIISFKEMNPPDNIKDTKSDIIFFIRSVPGADNKIQFESSSYEGYFLACEKERDLFKLILKKEDELGDRSIMFTVQNED

SEQ ID NO: 70 - protein, 157 aa, synthetic construct
HFGKLESKLSVIRNLNGQVLFIDQGNRPLFGDMEASPCRDNAPRTIFIISFYKDSQPRGMAVTISVKCEKISTLSCENKIISFKEMNPPDNIKDTKSDIIFFIRSVPGADNKLQFESSSYEGYFLACEKERDLFKLILKKEDELGDRSIMFTVQNED

SEQ ID NO: 71 - protein, 157 aa, synthetic construct
HFGKLESKLSVIRNLNGQVLFIDQGNRPLFTDMTSSDCRDKAPRTIFIISFYKDSQPRGLAVTISVKCEKISTLSCENKIISFKEMNPPDNIKDTKSDIIFFLRSVPGPDNKFQFESSSYEGYFLACEKERDLFKLILKKEDELGDRSIMFTVQNED

SEQ ID NO: 72 - protein, 157 aa, synthetic construct
HFGKLESKLSVIRNLNGQVLFIDQGNRPLFADMESNRCRDSAPRTIFIISMYKDSQPRGFAVTISVKCEKISTLSCENKIISFKEMNPPDNIKDTKSDIIFFLRSVPGHDNKIQFESSSYEGYFLACEKERDLFKLILKKEDELGDRSIMFTVQNED

SEQ ID NO: 73 - protein, 157 aa, synthetic construct
YFGKLESKLSVIRNLNGQVLFIDQGNRPLFTDMTASPCRDNAPRTIFIISFYKDSQPRGLAVTISVKCEKISTLSCENKIISFKEMNPPDNIKDTKSDIIFFLRSVPGHDNKIQFESSSYEGYFLACEKERSLFKLILKKEDELGDRSIMFTVQNED

SEQ ID NO: 74 - protein, 157 aa, synthetic construct
DFGKLESKLSVIRNLNDQVLFIDQGNRPLFADMKSNVCRANAPRTIFIISMYKDSQPRGMAVTISVKCEKISTLSCENKIISFKEMNPPDNIKDTKSDIIFFIRSVPGPDNKLQFESSSYEGYFLACEKERDLFKLILKKEDELGDRSIMFTVQNED

SEQ ID NO: 75 - protein, 157 aa, synthetic construct
HFGKLESKLSVIRNLNGQVLFIDQGNRPLFGDMEASPCRAKAPRTIFIISIYKDSQPRGFAVTISVKCEKISTLSCENKIISFKEMNPPDNIKDTKSDIIFFLRSVPGHDNKFQFESSSYEGYFLACEKERSLFKLILKKEDELGDRSIMFTVQNED

SEQ ID NO: 76 - protein, 157 aa, synthetic construct
HFGKLESKLSVIRNLNGQVLFIDQGNRPLFADMASNRCRANAPRTIFIISMYKDSQPRGFAVTISVKCEKISTLSCENKIISFKEMNPPDNIKDTKSDIIFFIRSVPGPDNKFQFESSSYEGYFLACEKERDLFKLILKKEDELGDRSIMFTVQNED

SEQ ID NO: 77 - protein, 157 aa, synthetic construct
YFGKLESKLSVIRNLNDQVLFIDQGNRPLFADMKAKACRSNAPRTIFIISFYKDSQPRGFAVTISVKCEKISTLSCENKIISFKEMNPPDNIKDTKSDIIFFLRSVPGADNKIQFESSSYEGYFLACEKERDLFKLILKKEDELGDRSIMFTVQNED

SEQ ID NO: 78 - protein, 157 aa, synthetic construct
HFGKLESKLSVIRNLNHQVLFIDQGNRPLFTDMADNACRDNAPRTIFIISFYKDSQPRGLAVTISVKCEKISTLSCENKIISFKEMNPPDNIKDTKSDIIFFIRSVPGDDNKMQFESSSYEGYFLACEKERDLFKLILKKEDELGDRSIMFTVQNED

SEQ ID NO: 79 - protein, 157 aa, synthetic construct
YFGKLESKLSVIRNLNGQVLFIDQGNRPLFTDMKSNLCRSNAPRTIFIISFYKDSQPRGIAVTISVKCEKISTLSCENKIISFKEMNPPDNIKDTKSDIIFFIRSVPGDDNKIQFESSSYEGYFLACEKERDLFKLILKKEDELGDRSIMFTVQNED

SEQ ID NO: 80 - protein, 157 aa, synthetic construct
HFGKLESKLSVIRNLNGQVLFIDQGNRPLFRDMAASHCRDSAPRTIFIISIYKDSQPRGFAVTISVKCEKISTLSCENKIISFKEMNPPDNIKDTKSDIIFFLRSVPGHDNKIQFESSSYEGYFLACEKERDLFKLILKKEDELGDRSIMFTVQNED

SEQ ID NO: 81 - protein, 157 aa, synthetic construct
YFGKLESKLSVIRNLNDQVLFIDQGNRPLFADMASNPCRYKAPRTIFIISMYKDSQPRGLAVTISVKCEKISTLSCENKIISFKEMNPPDNIKDTKSDIIFFLRSVPGADNKLQFESSSYEGYFLACEKERDLFKLILKKEDELGDRSIMFTVQNED

SEQ ID NO: 82 - protein, 157 aa, synthetic construct
HFGKLESKLSVIRNLNGQVLFIDQGNRPLFTDMASNHCRYNAPRTIFIISMYKDSQPRGLAVTISVKCEKISTLSCENKIISFKEMNPPDNIKDTKSDIIFFLRSVPGADNKIQFESSSYEGYFLACEKERDLFKLILKKEDELGDRSIMFTVQNED

SEQ ID NO: 83 - protein, 157 aa, synthetic construct
HFGKLESKLSVIRNLNGQVLFIDQGNRPLFADMTDNPCRSRAPRTIFIISFYKDSQPRGMAVTISVKCEKISTLSCENKIISFKEMNPPDNIKDTKSDIIFFIRSVPGHDNKFQFESSSYEGYFLACEKERDLFKLILKKEDELGDRSIMFTVQNED

SEQ ID NO: 84 - protein, 157 aa, synthetic construct
YFGKLESKLSVIRNLNGQVLFIDQGNRPLFTDMTASHCRSSAPRTIFIISLYKDSQPRGMAVTISVKCEKISTLSCENKIISFKEMNPPDNIKDTKSDIIFFLRSVPGHDNKFQFESSSYEGYFLACEKERDLFKLILKKEDELGDRSIMFTVQNED

SEQ ID NO: 85 - protein, 157 aa, synthetic construct
YFGKLESKLSVIRNLNGQVLFIDQGNRPLFTDMEYRLCRANAPRTIFIISFYKDSHPRGLAVTISVKCEKISTLSCENKIISFKEMNPPDNIKDTKSDIIFFLRSVPGDDNKLQFESSSYEGYFLACEKERDLFKLILKKEDELGDRSIMFTVQNED

SEQ ID NO: 86 - protein, 157 aa, synthetic construct
YFGKLESKLSVIRNLNGQVLFIDQGNRPLFTDMESSLCRDNAPRTIFIISLYKDSQPRGMAVTISVKCEKISTLSCENKIISFKEMNPPDNIKDTKSDIIFFLRSVPGADNKFQFESSSYEGYFLACEKERSLFKLILKKEDELGDRSIMFTVQNED

SEQ ID NO: 87 - protein, 157 aa, synthetic construct
YFGKLESKLSVIRNLNGQVLFIDQGNRPLFKDMEANDCRSSAPRTIFIISIYKDSQPRGLAVTISVKCEKISTLSCENKIISFKEMNPPDNIKDTKSDIIFFIRSVPGADNKMQFESSSYEGYFLACEKERDLFKLILKKEDELGDRSIMFTVQNED

SEQ ID NO: 88 - protein, 157 aa, synthetic construct
DFGKLESKLSVIRNLNDQVLFIDQGNRPLFADMKASACRANAPRTIFIISMYKDSQPRGLAVTISVKCEKISTLSCENKIISFKEMNPPDNIKDTKSDIIFFLRSVPGHDNKFQFESSSYEGYFLACEKERDLFKLILKKEDELGDRSIMFTVQNED

SEQ ID NO: 89 - protein, 157 aa, synthetic construct
YFGKLESKLSVIRNLNGQVLFIDQGNRPLFGDMTAKHCRARAPRTIFIISFYKDSQPRGMAVTISVKCEKISTLSCENKIISFKEMNPPDNIKDTKSDIIFFIRSVPGADNKFQFESSSYEGYFLACEKERDLFKLILKKEDELGDRSIMFTVQNED

SEQ ID NO: 90 - protein, 157 aa, synthetic construct
FFGKFESKLSVIRNLNGQVLFIDQGNRPLFTDMESKDCRDRAPRTIFIISFYKDSQPRGLAVTISVKCEKISTLSCENKIISFKEMNPPDNIKDTKSDIIFFLRSVPGHDNKLQFESSSYEGYFLACEKERDLFKLILKKEDELGDRSIMFTVQNED

SEQ ID NO: 91 - protein, 157 aa, synthetic construct
FFGKLESKLSVIRNLNGQVLFIDQGNRPLFADMASNHCRANAPRTIFIISLYKDSQPRGLAVTISVKCEKISTLSCENKIISFKEMNPPDNIKDTKSDIIFFIRSVPGHDNKMQFESSSYEGYFLACEKERDLFKLILKKEDELGDRSIMFTVQNED

SEQ ID NO: 92 - protein, 157 aa, synthetic construct
YFGKLESKLSVIRNLNGQVLFIDQGNRPLFADMTSKRCRDNAPRTIFIISLYKDSQPRGFAVTISVKCEKISTLSCENKIISFKEMNPPDNIKDTKSDIIFFIRSVPGHDNKIQFESSSYEGYFLACEKERDLFKLILKKEDELGDRSIMFTVQNED

SEQ ID NO: 93 - protein, 157 aa, synthetic construct
LFGKHESKLSVIRNLNGQVLFIDQGNRPLFGDMESSPCRYNAPRTIFIISFYKDSQPRGLAVTISVKCEKISTLSCENKIISFKEMNPPDNIKDTKSDIIFFIRSVPGHDNKMQFESSSYEGYFLACEKERDLFKLILKKEDELGDRSIMFTVQNED

SEQ ID NO: 94 - protein, 157 aa, synthetic construct
YFGKLESKLSVIRNLNAQVLFIDQGNRPLFTDMTASPCRSSAPRTIFIISLYKDSQPRGLAVTISVKCEKISTLSCENKIISFKEMNPPDNIKDTKSDIIFFLRSVPGPDNKIQFESSSYEGYFLACEKERDLFKLILKKEDELGDRSIMFTVQNED

SEQ ID NO: 95 - protein, 157 aa, synthetic construct
YFGKLESKLSVIRNLNGQVLFIDQGNRPLFADMTDSDCRDNAPRTIFIISMYKDSQPRGMAVTISVKCEKISTLSCENKIISFKEMNPPDNIKDTKSDIIFFLRSVPGHDNKMQFESSSYEGYFLACEKERDLFKLILKKEDELGDRSIMFTVQNED

SEQ ID NO: 96 - protein, 157 aa, synthetic construct
YFGKLESKLSVIRNLNGQVLFIDQGNRPLFADMTSSDCRDNAPRTIFIISFYKDSQPRGMAVTISVKCEKISTLSCENKIISFKEMNPPDNIKDTKSDIIFFLRSVPGHDNKMQFESSSYEGYFLACEKERDLFKLILKKEDELGDRSIMFTVQNED

SEQ ID NO: 97 - protein, 157 aa, synthetic construct
YFGKLESKLSVIRNLNGQVLFIDQGNRPLFADMESSDCRDNAPRTIFIISFYKDSQPRGLAVTISVKCEKISTLSCENKIISFKEMNPPDNIKDTKSDIIFFLRSVPGHDNKMQFESSSYEGYFLACEKERDLFKLILKKEDELGDRSIMFTVQNED

SEQ ID NO: 98 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 99 - DNA, 7809 bp, synthetic construct
  source 1..7809: mol_type = other DNA
gtcgacggacccccgggaaagatttaatacgactcactatagggcgggacagaartgatctgtgagagactcatctagttcataccctaggtgacccrgggggtggcatgggggragattagagatcccagtctggtatcctctggagagtaggagtcccaggagctgaaggtttctggccactgaactttggctaaagcagaggtgtcacagctgctcaagattccctggttaaaaagtgaaagtgaaatagagggtcggggcagtgctttcccagaaggattgctcggcatcctgcccttcccagaagcagctctggtgctgaagagagcactgcctccctgtgtgactgggtgagtccarattctcrctttgggtctcaattttgccttccctaatgaaggggtaagattggactaggtaagcatcrtacaaccatttgtggtcatgagagctggggrggggaaggattgtcacrtgacccccccagctctgtttctaagtgctgaaagagctccaggctatgctacgggaggagaagccagctactgaggaaaagccagctactgagaaaaagcgggagtggtttaccattctcctcccccacctttcaccagagaagaggacgttgtcacacataaagagccaggctcaccagctcctgacgcargcatcatgaccatgagacacaactggacaccaggtaggccttggggctacgcatgggcaggcggggtagggtgaggtctatgaacagaatggagcaargggctaacccggagccttcacrccaaggcaaaccacccagcgcacctggrgcrgrtgctttaagaacctgggcagatarrgragctctggctccagtctaaagcttctcrgtacrctgtrcaataaagggctaaggggrgggtgctgaggggtccctcttcccgctctgattccctggctagaacccagacatcrcrgggcrggagrtacatccttacccgggcagcccactctgtctccagagccgctgacctgtaactgtcctttcctcagacctcagccctttgtgggtcctgctcctgtgtgcccacgtcgtcactctcctggtcagagccacacctgtctcgcagaccaccacagctgccactgcctcagttagaagcacaaaggacccctgcccctcccagcccccagtgttcccagcagctaagcagtgtccagcartggaagtgacctggccagaggtggaagtgccactgagraagaagcacagrggtggagggtgggcratgggcacagaggttcccagggrcgggttgactcctgagcgccagtccccttcrgcccatgtaccaccagctgagccagctgggctgagcacgcaccattctccctccccaacccagtgtcatgggrgcaggcrrggcgcagcrcccaagatgctccctatcaaataggacagagaactcaagacataagtaatggtcacaggacctcccagagccttggttgcagtggaccccaaggccagcccctccacccagagcctctggcctctggccatctcagaggagcagcagccatccagcactgcctctgtcacctgggcrcccaagtcaccgaggctgggcactagaaaaggtcatcctgaggagacaggttcagaagaggartcatcacgtgaaccaaggaccattcctcacattccccgtgtttagggctagggcctctcggagacaactgcacrtctgtaacggacgrtcccacctaggtggtgtgcagagcagttctctaggttccagatgcatggggactggggggagctggcagagagggcacagcagagcagggraggggaagggcctgctcttctgaagagcraactgctgccrgtgtccctagatggaacgctgagcttatcctgtgtggcctgcagccgcttccccaacttcagcatcctctactggcrgggcaatggttccttcattgagcacctcccaggccgactgtgggaggggagcaccaggtgagggtcgcagcagccaggtgggtgggaagaagcctrctgcggccrtctcargacctrtccttcccttccgctccagccgggaacgtgggagcacaggtacgcagctgtgcaaggccttggtgctggagcagctgacccctgccctgcacagcaccaacttctcctgtgtgctcgtggaccctgaacaggttgtccagcgtcacgtcgrcctggcccagctctgggtgaggagcccaaggagaggcctccaggaacaggaggagctctgcttccatatgtggggaggaaagggtgggctctgccagagcagcctgtgaactaatgcccagcattcctcaaggtcagccagacaaaaaggaacttaggtcttgggcagaggaggtgtagcctggggcaaagtgatgagatgtccctcctttccttggcctgatccttgtctgccttcacttccctaggctgggctgagggcaaccttgccccccacccaagaagccctgccctccagccacagcagtccacagcagcagggttaagactcagcacagggccagcagcagcacaaccttgaccagagcttgggtcctacctgtctacctggagtgaacagtccctgactgcctgtaggctgcgtggatgcgcaacacaccccctccttctctgctttgggtcccttctctcaccaaattcaaactccattcccacctacctagaaaatcacagcctccttataatgcctcctcctcctgccattctctctccacctatccattagccttcctaacgtcctactcctcacactgctctactgctcagaaaccaccaagactgttgatgccttagccttgcactccagggccctacctgcatttcccacatgactttctggaagcctcccaactattcttgcttttcccagacagctcccactcccatgtctctgctcatttagtcccgtcttcctcaccgccccagcaggggaacgctcaagcctggttgaaatgctgcctcttcagtgaagtcarcctctttcagctctggccgcattctgcagacttcctatcttcgtgctgtatgttttttttttcccccttcactctaatggactgttccagggaagggatgggggcagcagctgcttcggatccacactgtatctgtgtcatccccacatgggtccrcataaaggattattcaatggaggcatcctgacatctgttcatttaggcttcagttccactcccaggaactttgcctgtcccacgagggagtatgggagagatggactgccacacagaagcrgaagacaacacctgcttcaggggaacacaggcgcttgaaaaagaaaagagagaacagcccataatgctccccgggagcagaggccactaatggagagtgggaagagcctggaaagatgtggcctcaggaaaagggatgagagaaaggaggtggtatggaagactcagcaggaacaaggtaggcttcaaagagcctatattcctctttttcccacaccgatcaagtcaactcagtactcacgggagaaaaatagactttatttacaagtaataacatttagaaaagatccarccccggcccttaaaaaccttcccatcactccaaatcccaccccagtgcaagtctggggaaggtagggtgtgagctgctgctgaaggctgtcccccaaccccactcctgagacacagggcccatccgtcctgggaaagagcatcctctggcaggrgctcccaccaggtcagacccagtcctggacttcaagagtgagggcccctgctgggcccagccaccaggacagcaggaaccagggcctactcctcttatggtcccttctagatccagaggctaagaggaagactggccaggcccaaggacccagccatcaaaaccagcctcaaatctggttgtgatggagaagtgactttgctttaagaaaaaaggaggcaaggtagggagagcgcccacactgtccatgctccaggccccctgggccagctccgagaaggcgccagtgaaggaccagggaccaggccagggtgcgggcaggcatcactgtctctaggggttrggctactgttggccrgggagctgagagaaggcactgagagggacagtaggcggaggaccaggrgacggcagcarcggggacacaggtggggccactcactggractggccctttagtgctttgcctgaaagagacacagtcacatggccagatgagaacttgcgatactagccrgcacccactggctgggaagarctcttcctgctcccacgcccctgtctggatcccctcccttgtgagccccagggttatcagtrgctggctgtgcctgagcagctctgggtgctctccatgagaarggggccatctgtcttctctccttggagaggagctaccaggacagggacacctcttaccccacaccctccagcagcctggcgtggccccatcttggatgctacttggtggggcggtctggggggtgcccatgctctcatcgggtttccctcccccatcctgccagtgcctctaccttgcccttggctcgaggggtggcaccaatggcggcagcagtggcggcgctggctgtggtggrggcaatgcgcggagaacggcgggttccactgcgagtgttgggggaagccttggacagggccttctttgaggctccccgccgcagaaggctgttccctagcttcttgggtgtgttgaggargcrgaaggccatcgactggcgccggtcagcctgcaaggaagggctgtcagaccgggagacccaargctgccttcccaggccagcgtgctgtgccacgctgtaccagcaaggtcccgccagggcgtcgcttcatcccccttcagccccagcctcacctgtrragtagaagctggagctgcrttcrtctgggcctcagtagtgctctgtttgcgcccttcatgtcggtctcggggagtcatggggcgtgggaaacagctggtggccttcttagactatggagaagaggacagttaggcagacagragcaagaggagtcacatctgaagccaggtgtcttgtcctctcagagctgagtggaccttgraagtcaacgtgcaacctgctccccttcccaactctgggccagatccttcccttcccaacagttcccatccatgggtcaggcccttggagagagggaaagagagggggaagtgagggaaggagagagaaggctccctttagtccttggtgagctgggcctgacctgagcacagtgctggagraacacccaggagccaccgcgcctacctcaggagttccagggccctggtggggctctagggagacccgtttgcgctgctgccgggtggtgatgccagtgccctcggctatctggattggctgcatgctggctcggcgcagggtctcttgggggtctccagttttcatctcctcatctgtgatggtgcccaggctcagggaaggctgcatgggtggaagaggtggtcagtggaccatagctgtatggagatggaggaggacctggggctgttccagaactctacactcgcccgacacttatggtcgggacccttcctgcctacgaggtagaaagacacaagcctcctttcctgttctgctttctacctaagccctgggcaaatggcacaagcagtgcagtcctgaccagattcctctctgagctcctgcctacccccagggacttcacccctgagtgccctccagctgtctgttccacctggaacatgagaaggtcaccccttcccctcttcggccagtcagtgatccagggccctagtgctcaggctagarcagcaggtgggartccaaggaagggcagggargggaggccctgcacagtgaccccaggcctcaccctggactccagggatagcaggtcttcagatgtggggggcacactcgattgcgctgctgcagctctgcaatgcggttccagtcarccagctgctcaggctcatcctggcaagtgcccatgtagaagctgttccttcctgtggaaggcagggaagtgggaacaaatgagcctggagtcggcaggtcacctcctggccctggcatcttgccagcctttgctgccacctaccccataaacttgaagcccggcacaccagtctgattcagtgccgcaggtgcaggagtacggcacacagactatttctatcctaggggcttgctcaccaccttctccctggagagggcagaagaggtcacacgcagagactgctactacatcttattcacctgccaaggcttggtggccaacacccagaggaacaaattaaggaccgggaattaattcccaggggctccctggtgcccaaaggacaagagcttccaagaagagtctggccagcctggcctttccagcagcccatcaccgcctgagaagggcarggaggactccccacagctaagrgrcacaartgtgctgggaatcccgggcccttaactctggctaagagtgcccccaacacagccagcccctagatgggcaggtaaggaaggccctgaggctgcaggaaggaggggcaggtggagctggatggtagcaaggaggccagccttggatttttaaaaagctttcctcttttccctgtgccacgatccaccttccagtctaattttggggtatagtaagtccctgtagtcccctcacctggaggggccccactggacaccccggcctgggaacgacgagcagaactgcgagtggtggggcggtagccaggcaagctgagcagggctgagttgccataatcgggagaacccaggcgagctagagactgagtagaggaggtggcrcgcaggctagcctgggaagcaggagcagaccgcgtgcrgtagaacgatgagttggcgctgtctggctcttccacatctagcttctggaagacagagtgaarcrgttgcagtgtacagrccctggcactgtacagaagcttcccattcccttccgaagcccrcagatcccacggcacatccatgtattcccaacrgcrttgcaaaggrccrraaagrgtgrgrctgcaagaaargggccrrgtcgacagaagccctcacaaggtggtgctgargttgtcaagactcttctacgcatttttttcatggagtctatrcaraatgctttgaggtagggaatgcagagtgtttatcggcccatrttggagatgaagtgcaaagaaataaagtgactagccccaaarcacactgctaggaagtatcagagctggggcraggccccargtctcctgactagtcaggctcatcccacagcctctgctgtccctcagtccaaacttccagggcccttaccatgttccagaacttcccccaacttcttggtagcagggggcaccctaaacacacaggtcccccctgctgtaccaggggccccctctcccctcctcccaaacctccccttcaagatgtggaaacaaaggcaagggcctgcagcctgtcaggcagtccactgggcagcaacaatgcctctcagctgcatggggcatgctgggaggcacaggatgggctgcagctrcgccacgttctctcccttcaccctgcacaggctcagtgctacgcatggagagaatgcragccttagtcaggaggcagggatctaatccragccctgcctttttcttcagaagtgcccttaaccaagtcactgccctttttaagaccrctcagctttcccactgtaacatggactggctgctcatccctccctgctccrgactgagrgcccag

SEQ ID NO: 100 - protein, 46 aa, synthetic construct
TPVSGINTTTAATASVRSTKDPCPSGINPPVFPAAKGINCPALEVT

SEQ ID NO: 101 - protein, 422 aa, synthetic construct
MRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQGRSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - DNA, 3383 bp, synthetic construct
  source 1..3383: mol_type = other DNA
gtaaagtagaaatgaatttatttttctttgcaaactaagtatctgcttgagacacatctatctcaccattgtcagctgaggaaaaaaaaaaatggttctcatgctaccaatctgccttcaaagaaatgtggactcagtagcacagctttggaatgaagatgatcataagagatacaaagaagaacctctagcaaaagatgcttctctatgccttaaaaaattctccagctcttagaatctacaaaatagactttgcctgtttcattggtcctaagattagcatgaagccatggattctgttgtagggggagcgttgcataggaaaaagggattgaagcattagaattgtccaaaatcagtaacacctcctctcagaaatgctttgggaagaagcctggaaggttccgggttggtggtggggtggggcagaaaattctggaagtagaggagataggaatgggtggggcaagaagaccacattcagaggccaaaagctgaaagaaaccatggcatttatgatgaattcagggtaattcagaatggaagtagagtaggagtaggagactggtgagaggagctagagtgataaacagggtgtagagcaagacgttctctcaccccaagatgtgaaatttggactttatcttggagataatagggttaattaagcacaatatgtattagctagggtaaagattagtttgttgtaacaaagacatccaaagatacagtagctgaataagatagagaatttttctctcaaagaaagtctaagtaggcagctcagaagtagtatggctggaagcaacctgatgatattgggacccccaaccttcttcagtcttgtacccatcatcccctagttgttgatctcactcacatagttgaaaatcatcatacttcctgggttcatatcccagttatcaagaaagggtcaagagaagtcaggctcattcctttcaaagactctaattggaagttaaacacatcaatccccctcatattccattgactagaatttaatcacatggccacaccaagtgcaaggaaatctggaaaatataatctttattccaggtagccatatgactctttaaaattcagaaataatatatttttaaaatatcattctggctttggtataaagaattgatggtgtggggtgaggaggccaaaattaagggttgagagcctattattttagttattacaagaaatgatggtgtcatgaattaaggtagacataggggagtgctgatgaggagctgtgaatggattttagaaacacttgagagaatcaataggacatgatttagggttggatttggaaaggagaagaaagtagaaaagatgatgcctacatttttcacttaggcaatttgtaccattcagtgaaatagggaacacaggaggaagagcaggttttggtgtatacaaagaggaggatggatgacgcatttcgttttggatctgagatgtctgtggaacgtcctagtggagatgtccacaaactcttctacatgtggttctgagttcaggacacagatttgggctggagatagagatattgtaggcttatacatagaaatggcatttgaatctatagagataaaaagacacatcagaggaaatgtgtaaagtgagagaggaaaagccaagtactgtgctggggggaatacctacatttaaaggatgcagtagaaagaagctaataaacaacagagagcagactaaccaaaaggggagaagaaaaaccaagagaattccaccgactcccaggagagcatttcaagattgaggggataggtgttgtgttgaattttgcagccttgagaatcaagggccagaacacagcttttagatttagcaacaaggagtttggtgatctcagtgaaagcagcttgatggtgaaatggaggcagaggcagattgcaatgagtgaaacagtgaatgggaagtgaagaaatgatacagataattcttgctaaaagcttggctgttaaaaggaggagagaaacaagactagctgcaaagtgagattgggttgatggagcagttttaaatctcaaaataaagagctttgtgcttttttgattatgaaaataatgtgttaattgtaactaattgaggcaatgaaaaaagataataatatgaaagataaaaatataaaaaccacccagaaataatgatagctaccattttgatacaatatttctacactcctttctatgtatatatacagacacagaaatgcttatatttttattaaaagggattgtactatacctaagctgctttttctagttagtgatatatatggacatctctccatggcaacgagtaattgcagttatattaagttcatgatatttcacaataagggcatatctttgccctttttatttaatcaattcttaattggtgaatgtttgtttccagtttgttgttgttattaacaatgttcccataagcattcctgtacaccaatgttcacacatttgtctgattttttcttcaggataaaacccaggaggtagaattgctgggttgatagaagagaaaggatgattgccaaattaaagcttcagtagagggtacatgccgagcacaaatgggatcagccctagataccagaaatggcactttctcatttccccttgggacaaaagggagagaggcaataactgtgctgccagagttaaatttgtacgtggagtagcaggaaatcatttgctgaaaatgaaaacagagatgatgttgtagaggtcctgaagagagcaaagaaaatttgaaattgcggctatcagctatggaagagagtgctgaactggaaaacaaaagaagtattgacaattggtatgcttgtaatggcaccgatttgaacgcttgtgccattgttcaccagcagcactcagcagccaagtttggagttttgtagcagaaagacaaataagttagggatttaatatcctggccaaatggtagacaaaatgaactctgagatccagctgcacagggaaggaagggaagacgggaagaggttagataggaaatacaagagtcaggagactggaagatgttgtgatatttaagaacacatagagttggagtaaaagtgtaagaaaactagaagggtaagagaccggtcagaaagtaggctatttgaagttaacacttcagaggcagagtagttctgaatggtaacaagaaattgagtgtgcctttgagagtaggttaaaaaacaataggcaactttattgtagctacttctggaacagaagattgtcattaatagttttagaaaactaaaatatatagcatacttatttgtcaattaacaaagaaactatgtatttttaaatgagatttaatgtttattgtag

SEQ ID NO: 103 - protein, 196 aa, synthetic construct
MAAEPVEDNCINFVAMKFIDNTLYFIAAEDDEENLESDYFGKLESKLSVIRNLNDQVLFIDQGNRPLFEDMTDSDCRDDNAPRTIFIISMYKDSQPRGMAVTISVKCEKISTLSCENKIISFKEMNPPDNIKDTKSDIIFFERSVPGHDNKMQFESSSYEGYFLACEKERDLFKLILKKEDELGDRSIMFTVQNED

SEQ ID NO: 104 - protein, 158 aa, synthetic construct
YFGKLESKLSVIRNLNDQVLFIDQGNRPLFEDMTDSDCRDDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 105 - protein, 147 aa, synthetic construct
IRNLNDQVLFIDQGNRPLFEDMTDSDCRDDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 106 - DNA, 1103 bp, synthetic construct
  source 1..1103: mol_type = other DNA
cctttgctcccctggcgactgcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatacaaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattca

SEQ ID NO: 107 - protein, 189 aa, synthetic construct
MAAEPVEDNCINFVAMKFIDNTLYFIENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 108 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED